(19) United States
(12) Patent Application Publication
(10) Pub. No.: US 2007/0243531 A1
(43) Pub. Date: Oct. 18, 2007

(54) Methods of Diagnosing Colorectal Cancer and Reagents Therefor

(76) Inventor: Maija Kohonen-Corish, Northwood (AU)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 10/586,978

(22) PCT Filed: Jan. 24, 2005
(86) PCT No.: PCT/AU05/00077

§ 371 Date: Feb. 16, 2007

Related U.S. Application Data
(60) Provisional application No. 60/540,662, filed on Jan. 29, 2004.

(30) Foreign Application Priority Data
Jan. 23, 2004 (AU) 2004900340

(51) Int. Cl. C12Q 1/68; C07H 21/04

(52) U.S. Cl. 435/6; 436/64; 536/24.31; 536/24.33

(57) ABSTRACT

The present invention relates to assays for diagnosing colorectal cancer or a predisposition therefore or the neoplastic potential of a colorectal polyp wherein hypermethylation of an MCC gene promoter is determined. The invention provides various multi-analyte diagnostic tests and reagents for performing these diagnostic/predictive assays.

### FIELD OF THE INVENTION

The present invention relates to methods for the diagnosis and treatment of cancer, particularly colorectal cancer. More particularly, the present invention provides methods for the diagnosis and treatment of colorectal cancer in a subject comprising determining the degree of methylation of nucleic acid that regulates expression of a tumor suppressor gene in a sample from the subject.

### BACKGROUND TO THE INVENTION

1. General

As used herein the term “derived from” shall be taken to indicate that a specified integer is obtained from a particular source albeit not necessarily directly from that source.

Unless the context requires otherwise or specifically stated to the contrary, integers, steps, or elements of the invention recited herein as singular integers, steps or elements clearly encompass both singular and plural forms of the recited integers, steps or elements.

The embodiments of the invention described herein with respect to any single embodiment shall be taken to apply mutatis mutandis to any other embodiment of the invention described herein.

Throughout this specification, unless the context requires otherwise, the word “comprise”, or variations such as “comprises” or “comprising”, will be understood to imply the inclusion of a stated step or element or integer or group of steps or elements or integers but not the exclusion of any other step or element or integer or group of elements or integers.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features. The present invention clearly encompasses combinations of any one or more of the assay formats described herein.

The present invention is not to be limited in scope by the specific examples described herein. Functionally equivalent products, compositions and methods are clearly within the scope of the invention, as described herein.

The present invention is performed without undue experimentation using, unless otherwise indicated, conventional techniques of molecular biology, microbiology, virology, recombining DNA technology, peptide synthesis in solution, solid phase peptide synthesis, and immunology. Such procedures are described, for example, in the following texts that are incorporated herein by reference:
1. Sambrook, Fritsch & Maniatis, Molecular Cloning: A Laboratory Manual, Cold Spring Harbor Laboratories, New York, Second Edition (1989), whole of Vols I, II, and III;
2. DNA Cloning: A Practical Approach, Vols. I and II (D. N. Glover, ed., 1985), IRL Press, Oxford, whole of text;
3. Oligonucleotide Synthesis: A Practical Approach (M. J. Gait, ed., 1984) IRL Press, Oxford, whole of text, and particularly the papers therein by Gait, pp1-22; Atkinson et al, pp35-81; Sproat et al., pp 83-115; and Wu et al., pp 135-151;
4. Nucleic Acid Hybridization: A Practical Approach (B. D. Hames & S. J. Higgins, eds., 1985) IRL Press, Oxford, whole of text;
5. Perbal, B., A Practical Guide to Molecular Cloning (1984).

This specification contains nucleotide and amino acid sequence information prepared using PatentIn Version 3.1, presented herein after the claims. Each nucleotide sequence is identified in the sequence listing by the numeric indicator <210> followed by the sequence identifier (e.g. <210>1, <210>2, <210>3, etc). The length and type of sequence (DNA, protein (PRT), etc), and source organism for each nucleotide sequence, are indicated by information provided in the numeric indicator fields <211>, <212> and <213>, respectively. Nucleotide sequences referred to in the specification are defined by the term “SEQ ID NO:”, followed by the sequence identifier (eg. SEQ ID NO: 1 refers to the sequence in the sequence listing designated as <400>1).

The designation of nucleotide residues referred to herein are those recommended by the IUPAC-IUB Biochemical Nomenclature Commission, wherein A represents Adenine, C represents Cytosine, G represents Guanine, T represents thymine, Y represents a pyrimidine residue, R represents a purine residue, M represents Adenine or Cytosine, K represents Guanine or Thymine, S represents Guanine or Cytosine, W represents Adenine or Thymine, H represents a nucleotide other than Guanine, B represents a nucleotide other than Adenine, V represents a nucleotide other than Thymine, D represents a nucleotide other than Cytosine and N represents any nucleotide residue.

2. Description of the Related Art

Cancer is a multi-factorial disease and major cause of morbidity in humans and other animals, and deaths resulting from cancer in humans are increasing and expected to surpass deaths from heart disease in future. Carcinomas of the lung, prostate, breast, colon, pancreas, and ovary are major contributing factors to total cancer death in humans. With few exceptions, metastatic disease from carcinoma is fatal. Even if patients survive their primary cancers, recurrence or metastases are common.

It is widely recognized that simple and rapid tests for solid cancers or tumors have considerable clinical potential. Not only can such tests be used for the early diagnosis of cancer but they also allow the detection of tumor recurrence following surgery and chemotherapy. A number of cancer-specific blood tests have been developed which depend upon the detection of tumor-specific antigens in the circulation (Catalona, W. J., et al., 1991, “Measurement of prostate-specific antigen in serum as a screening test for prostate cancer”, N. Engl. J. Med. 324, 1156-1161; Barrenetxea, G., et al., 1998, “Use of serum tumor markers for the diagnosis and follow-up of breast cancer”, Oncology, 55, 447-449; Cairns, P., and Sidreansky, D., 1999, “Molecular methods for the diagnosis of cancer”. Biochim. Biophys. Acta. 1423, C 11-C 18).

Colorectal cancer is the second most frequently diagnosed malignancy in the United States as well as the second most common cause of cancer death. The five-year survival rate for patients with colorectal cancer detected in an early localized stage is about 92%. However, only about 37% of colorectal cancer is diagnosed at an early localized stage. The survival rate of patients suffering from colorectal cancer drops to about 64% if the cancer is allowed to spread to adjacent organs or lymph nodes, and to 7% in patients with distant metastases.

Early symptoms of colorectal cancer, whilst not conclusive of the disease include bleeding from the rectum, diarrhea or constipation lasting for more than two weeks, bowel habits that oscillate between diarrhea and constipation, pain in the abdomen or rectum, incomplete bowel movements, unexplained weight loss, unusually low red blood cell counts or anemia, paleness, fatigue, or a yellowish coloring of the skin or whites of the eyes.

Diagnosis of colorectal cancer is known to utilize screening assays for fecal occult blood, sigmoidoscopy, colonoscopy and double contrast barium enemas.

The prognosis of colorectal cancer is directly related to the degree of penetration of the tumor through the bowel wall and the presence or absence of nodal involvement. As a consequence, early detection and treatment of colorectal cancer are highly desirable.

There is evidence that colorectal cancers may develop by distinct pathways having different clinical, pathological and molecular characteristics. For example, chromosomal instability or microsatellite instability (MSI) may occur. Methylation of CpG islands in DNA occurs to varying degrees in colorectal cancers having a high frequency of microsatellite instability Toyota et al., Proc. Natl Acad. Sci USA 97, 710-715, 2000), and in serrated adenoma (Park et al., Am. J. Pathol. 162, 815-822, 2003), and at low frequency in certain hyperplastic polyps (Chan et al., Am. J. Pathol. 160, 529-536, 2002). CpG island methylation phenotype (CIMP) may indicate microsatellite instability. To determine CIMP, the degree of methylation of a plurality of anonymous CpG island sequences (MINTs), not a single marker, is determined. Four MINTs (i.e., MINT 1, MINT 2, MINT 12 and MINT 31) are sufficiently sensitive and reliable to determine a CIMP when used together (Toyota et al., Proc. Natl Acad. Sci USA 97, 710-715, 2000; Hawkins et al., Gastroenterol. 122, 1376-1387, 2002; Whitehall et al., Cancer Res. 62, 6011-6014, 2002). However, these MINTs are not used routinely to diagnose/prognose colorectal cancer, let alone at an early stage.

Treatment regimens are determined by the type and stage of the cancer, and include surgery, radiation therapy and/or chemotherapy. Recurrence following surgery (the most common form of therapy) is a major problem and is often the ultimate cause of death.

Stage 0 (Carcinoma In Situ)

A stage 0 colorectal cancer or “carcinoma in situ” is a pre-cancerous condition, usually found in a polyp. Epithelial lesions with glandular serration include aberrant crypt foci (ACF), hyperplastic polyps (HP), mixed polyps (MP), serrated adenomas (SA) (e.g., Longacre et al., Am. J. Surg. Pathol. 14, 524-537, 1990) and sessile serrated adenomas (SSA; Arthur et al., J. Clin. Pathol. 21, 735-743, 1968). It is generally thought that MP and SA arise from HP.

Not all polyps necessarily transform into a clinically significant polyp or give rise a malignant condition. For example, whilst the existence of MP suggests a transition from HP to SA (Lino et al., J. CLin. Pathol. 52, 5-9, 1999), ACF and HP are thought to be heterogeneous with respect to their clinical behaviour such that they do not always transform. On the other hand, hyperplastic polyposis arising from a so-called variant HP is increasingly being associated with colorectal cancer that represents an early stage at which MSI is over-represented (Jass et al.,Histopathol. 37, 295-301, 2000; Jeevaratnam et al., J. Pathol. 179, 20-25, 1996; Leggett et al., Am. J. Surg. Pathol. 25,177-184, 2001; Rashid et al., Gastroenterol. 119, 323-332, 2000). In fact, hyperplastic polyposis may represent the most severe manifestation of a predisposition to produce large serrated polyps with an increased risk of malignancy. Wynter et al., Gut 53, 573-580, 2004 suggest that ACF and traditional HP, are non-progressing lesions having little or no potential for neoplastic transformation, however those variant HP in which methylation of at least 3-4 CIMPs occurs may progress to SA or MP and eventually colorectal cancer. Wynter et al. also proposed that whether or not such CIMP-high polyps progressed depends on the additional presence of a BRAF gene mutation (or possibly a K-ras mutation), and loss of function of the DNA repair genes MGMT and MLH1.

Additionally, there appears to be some heterogeneity between traditional HP and variant HP. For example, HP representing hyperplastic polyposis are morphologically distinguishable by size, architecture, differentiation, and proliferation, from traditional HPs localized to the distal colon and rectum. (Torlakovic et al., Gastrolenterol. 110, 748-755, 1996). However, numbers of polyps and anatomical location within the colorectum are not indicators of a predisposition for malignancy, because those HPs giving rise to malignancy are not localized.

Thus, a subset of serrated polyps e.g., certain HPs, SA and MP, is implicated in early stage colorectal cancer e.g., hyperplastic polyposis.

A K-ras mutation has been detected at high frequency in ACF, (Chan et al., Am. J. Pathol. 160, 1823-1830, 2002; Jen et al., Cancer Res 54, 5523-5526, 1994), however occurs at a reduced frequency in hyperplastic polyposis (Otori et al., Gut 40, 660-663, 1997), SA (Ajioka et al., Gut 42, 680-684, 1998; Sawyer et al., Gut 51, 200-206, 2002), and sporadic colorectal cancer with high level MSI (Samowitz et al., Am. J. Pathol. 158, 1517-1524, 2001).

Mutation or loss of the tumor suppressor genes p53 and Smad4/DPC4A may also accompany the transition from normal colonic mucosa to metastatic carcinoma

To date, there is no sensitive, reliable and straightforward assay for detecting a predisposition for colorectal cancer or early stage diagnosis, in particular the detection of those colbrectal polyps having a potential for malignancy (i.e., transformation potential or clinical significance).

Stage I(A) Colorectal Cancer

A stage I(A) colorectal cancer is one that is confined to the lining of the colon and does not penetrate the wall of the colon into the abdominal cavity, and has not spread to any adjacent organs or local lymph nodes and cannot be detected in other locations in the body, as determined following surgical removal of the tumor. Stage I(A) colorectal cancer is generally curable by surgical resection. Depending on features of the cancer under the microscope, approximately 90% of patients are cured without evidence of cancer recurrence following treatment with surgery alone. A minority of patients with stage I colorectal cancer may experience recurrence, partly as a consequence of undetectable micro metastases outside the colon.

Stage II(B) Colorectal Cancer

A stage II (B) colorectal cancer is one that has penetrated the wall of the colon into the abdominal cavity, but does not invade any of the local lymph nodes and cannot be detected in other locations in the body, as determined by histopathology of resected colon.

Despite undergoing complete surgical removal of the cancer, 25-40% of patients with stage II colorectal carcinoma experience recurrence of their cancer, partly as a consequence of micro metastases.

Stage III(C) Colorectal Cancer

At stage III (C), the tumor has penetrated the wall of the colon into the abdominal cavity and invaded any of the local lymph nodes, but cannot be detected in other locations in the body, as determined by histopathology of resected colon.

Despite undergoing complete surgical removal of the cancer, half of patients with stage III colon carcinoma experience recurrence of their cancer, partly as a consequence of micro metastases.

Stage IV(D) Colorectal Cancer

A stage IV (D) colorectal cancer is one that has metastasized to distant locations in the body, which may include the liver, lungs, bones or other sites.

Patients diagnosed with stage IV colon cancer have been perceived to have few treatment options. Certain patients, however, can still be cured of their cancer, and others derive significant benefit from additional treatment. Patients with stage IV colon cancer can be broadly divided into two groups: those with cancer spread that is localized to a single site and those with more widespread cancer.

Hereditary Non-Polyposis Colorectal Cancer (HNPCC)

HNPCC accounts for about 2% of colorectal cancer cases, and is characterized by the tendency to early onset of cancer and the development of other cancers, particularly those involving the endometrium, urinary tract, stomach, and biliary system. HNPCC is thought to be a consequence of mutations of one or more genes in the DNA mismatch repair (MMR) pathway, in particular the MSH2 and MLH1 genes. Loss of MMR activity is believed to contribute to cancer progression by virtue of cells accumulating other gene mutations and deletions, such as loss of the BAX gene which controls apoptosis, and the TGFβ2 receptor gene which controls cell growth.

Familial Adenomatous Polyposis (FAP)

Those skilled in the art will be aware that FAP is characterized by the early development of multiple colorectal adenomas that progress to cancer at a mean subject age of 44 years. FAP is believed to be caused by an inherited mutation in the adenomatous polyposis coli (APC) gene, thereby resulting in the loss of orderly replication, adhesion, and migration of colonic epithelial cells and leading to polyposis. Much attention has focussed on the detection of inherited and somatic mutations of the APC gene in colorectal cancer, especially as a diagnostic for FAP. U.S. Pat. No. 5,648,212 incorporated by reference disclosed several mutations within the APC gene as being diagnostic of colorectal cancer, and teach methods of diagnosis of colorectal cancer based upon those mutations.

Traverso et al., New England J. Med 346, 311-320, 2002, incorporated by reference, purified DNA from routinely collected stool samples and screened non-invasively for mutations in APC, by digital protein truncation. In particular mutations in codon 1303, codon 1412, codon 1435, codon 1489 and codon 1554 of the APC gene were detected. Stool samples from 28 patients having non-metastatic colorectal cancers, 18 subjects having adenoma of 1 cm diameter or larger, and 28 control patients, were studied and APC mutations were identified in 26 of 46 subjects with neoplasia, but none of the control samples.

Ulcerative Colitis

Ulcerative colitis predisposes affected individuals to about a 20-fold increased risk of developing cancer, associated with mutations in the p53 gene which may occur early, appearing even in histologically-normal tissue. The progression of the disease from ulcerative colitis to dysplasia/carcinoma without an intermediate polyp state suggests a high degree of mutagenic activity resulting from the exposure of proliferating cells in the colonic mucosa to the colonic contents.

Notwithstanding considerable research into therapies for the disease, colorectal cancer remains difficult to diagnose and treat effectively. Accordingly, there is a need in the art for improved methods for detecting and treating such cancers. There is a particular need to identify genes for which changes in expression may provide an early indicator of colorectal cancer or a predisposition for the development of colorectal cancer.

### SUMMARY OF THE INVENTION

In work leading up to the present invention, the inventors sought to develop new markers for the sensitive, reliable and straightforward diagnosis of colorectal cancer, especially for determining those early stage polyps of clinical significance or having transformation or neoplastic potential.

The inventors found that the 5-upstream region of a gene known previously to be mutated in colorectal cancer (i.e., MCC″) was differentially methylated in early stage and later stage colorectal cancers. In particular, the inventors gound that regions of the MCC gene that regulate its expression are hyper methylated, in samples derived from colorectal cancer subjects relative to comparable samples derived from suitable control subjects.

As exemplified herein for a cohort of 103 subjects having stage C colorectal cancer, aberrant hyper methylation of the MCC gene occurs in at least about 50% of cases. This aberrant methylation is is also correlated with reduced expression of the gene in subjects suffering from colorectal cancer. The present inventors showed that the promoter region of the MCC gene, and still more particularly the region between about position −600 and about position −139 relative to the translation start site of the MCC gene, is hyper methylated in samples derived from colorectal cancer subjects, and more preferably from about position −394 to about position −139 relative to the translation start site of the MCC gene. Within this region of the MCC gene promoter, 26 CpG sites were shown to be the most reliable indicators of reduced MCC gene expression or silencing.

Methylation of MCC also correlates positively with a number of clinicopathological variables in stage C colorectal cancer, in particular tumor grade i.e., degree of differentiation e.g., poorly differentiated grade 3 or moderately differentiated grade 2 or well-differentiated grade 1, and number of malignant lymph nodes, there being higher grade tumors and a higher number of malignant lymph nodes at stage C in subjects having hypermethylation in the MCC gene. Additionally, stage C tumors in the proximal colon are more frequently associated with MCC methylation suggesting to the inventors that they may arise from variant HPs or SSA rather than traditional HPs.

Based upon these data, the inventors designed novel nucleic acid probes for specifically detecting hyper methylated MCC gene promoter fragments in cell lines derived from colorectal cancer subjects, and showed that these nucleic acid probes also successfully detected hyper methylated MCC gene promoter fragments in samples derived from at least about 40% of colorectal patients having stage C colorectal cancers, but not in samples derived from comparable samples from normal tissues of the same subjects.

The inventors also examined the methylation status of the MCC gene promoter in carcinoma in situ, in particular HP and SA. As exemplified herein, MCC is hypermethylated in a significant proportion of HP, e.g., at least about 80% of HP and more preferably at least about 90% of HP or about 100% of HP.

The inventors also showed that methylation of MCC is not necessarily correlated to microsatellite instability. This contrasts with previous studies showing methylatioin of the DNA mismatch repair gene hMLH1.

These findings indicate a utility for MCC methylation as a marker for a number of diagnostic applications, including:
(i) detecting a precancerous lesion;
(ii) determining a predisposition for neoplastic transformation in a subject having colorectal polyps; and/or
(iii) determining the likelihood of progression of a colorectal polyp to SA and/or MP and/or colorectal cancer of any stage (e.g., progression of HP to SA and/or MP); and/or
(iv) determining the malignant potential of a colorectal polyp; and/or
(v) diagnosing hyperplastic polyposis; and/or
(vi) diagnosing colorectal cancer at any stage such as, for example, carcinoma in situ, Stage A, Stage B or Stage C.

Accordingly, the present invention provides a method of diagnosing colorectal cancer or hyperplastic polyposis in a subject, said method comprising determining the degree of methylation of nucleic acid that regulates expression of an MCC gene in a sample derived from the subject, wherein an enhanced degree of methylation in the sample relative to a suitable control sample is indicative of colorectal cancer or hyperplastic polyposis.

The present invention also provides a method for determining a predisposition for neoplastic transformation in a subject having colorectal lesions or polyps said method comprising determining the degree of methylation of nucleic acid that regulates expression of an MCC gene in a sample derived from a lesion or polyp wherein an enhanced degree of methylation in the sample relative to a suitable control sample is indicative of a predisposition for neoplastic transformation in the subject.

As used herein the term “predisposition for neoplastic transformation in a subject having colorectal polyps” shall be taken to mean that a colorectal polyp has neopolastic or malignant potential and/or is likely to progress to SA and/or MP and/or colorectal cancer of any stage. Accordingly, the term “method of determining a predisposition for neoplastic transformation in a subject having colorectal polyps” applies mutatis mutandis to such applications and indications.

It will also be apparent to the skilled artisan that the diagnostic/predictive tests described herein are useful for monitoring the efficacy of surgical resection, chemotherapy, radiation therapy undetaken for the purposes of treating a colorectal cancer, because the treatment should ablate the aberrant methylation pattern of a colorectal sample taken after treatment It will also be apparent that the invention is useful for determining recurrence of polyps having neoplastic potential or colorectal cancer.

Accordingly, the present invention provides a method of monitoring the efficacy of treatment for colorectal cancer or hyperplastic polyposis in a subject, said method comprising treating a subject in need of treatment for colorectal cancer or hyperplastic polyposis and determining the degree of methylation of nucleic acid that regulates expression of an MCC gene in a sample derived from the subject, wherein a degree of methylation in the sample comparable to that for a suitable control sample is indicative of effective treatment.

In a related embodiment, the present invention provides a method of monitoring the efficacy of treatment for colorectal cancer or hyperplastic polyposis in a subject, said method comprising treating a subject in need of treatment for colorectal cancer or hyperplastic polyposis and determining the degree of methylation of nucleic acid that regulates expression of an MCC gene in a sample derived from the subject, wherein a hyper methylation of the MCC promoter in the sample compared to the level of methylation for a suitable control sample indicates that treatment is not effective.

Preferred embodiments of the present invention are directed to multianalyte tests wherein the level of methylation of MCC and another gene selected from the group consisting of HLTF, APC, p16^{INK4a},p14^{ARF}, HPP1, hMLH1, MGMT, and combinations thereof is also determined. Accordingly, hypermethylation of MCC and the other analyte is indicative of colorectal cancer or hyperplastic polyposis or a predisposition of a lesion or polyp to neoplastic transformation.

Alternatively or in addition, the level of methylation of MCC and a CpG island methylation phenotype (CIMP) is determined using CIMP markers known in the art e.g., MINT 1, MINT 2, MINT 12 and MINT 31. Hypermethylation of MCC in combination with methylation of 3-4 CIMP markers is indicative of colorectal cancer or hyperplastic polyposis or a predisposition of a lesion or polyp to neoplastic transformation.

Alternatively, or in addition, the level of methylation of MCC and a mutation in the APC gene or DCC gene are determined. Hypermethylation of MCC in combination with a deletion or point mutation of APC or DCC are indicative of of colorectal cancer or hyperplastic polyposis or a predisposition of a lesion or polyp to neoplastic transformation.

Such multianalyte formats are particularly suited for all diagnostic and predictive applications described herein.

For example, the present invention also provides a method of diagnosing colorectal cancer or hyperplastic polyposis in a subject or determining a predisposition for neoplastic transformation in a subject having colorectal lesions or polyps, said method comprising (i) determining the degree of methylation of nucleic acid that regulates expression of an MCC gene in a sample derived from the subject; and (ii) determining the degree of methylation of at least one nucleic acid selected from the group consisting of a nucleic acid regulating expression of HLTF, a nucleic acid regulating expression of APC, a nucleic acid regulating expression of p16^{INKa}, a nucleic acid regulating expression of p14^{ARF}, a nucleic acid regulating expression of HPP1, a nucleic acid regulating expression of HMLH1, a nucleic acid regulating expression of MGMT, and combinations thereof in a sample derived from the subject, wherein an enhanced degree of methylation of the nucleic acid at (i) and at least one nucleic acid at (ii) relative to a suitable control sample is indicative of colorectal cancer or hyperplastic polyposis or a predisposition for neoplastic transformation of a lesion or polyp in the subject.

For example, the present invention also provides a method of diagnosing colorectal cancer or hyperplastic polyposis in a subject or determining a predisposition for neoplastic transformation in a subject having colorectal lesions or polyps, said method comprising (i) determining the degree of methylation of nucleic acid that regulates expression of an MCC gene in a sample derived from the subject; and (ii) determining a mutation such as a point mutation or deletion in a gene selected from the group consisting of APC and DCC, wherein an enhanced degree of methylation of the nucleic acid at (i) and a mutation at (ii) relative to a suitable control sample is indicative of colorectal cancer or hyperplastic polyposis or a predisposition for neoplastic transformation of a lesion or polyp in the subject

For example, the present invention also provides a method of diagnosing colorectal cancer or hyperplastic polyposis in a subject or determining a predisposition for neoplastic transformation in a subject having colorectal lesions or polyps, said method comprising (i) determining the degree of methylation of nucleic acid that regulates expression of an MCC gene in a sample derived from the subject; and (ii) determining the CpG island methylation phenotype of a sample derived from the subject using at least three MINT markers, wherein an enhanced degree of methylation of the nucleic acid at (i) and methylation of at least three MINT markers at (ii) relative to a suitable control sample is indicative of colorectal cancer or hyperplastic polyposis or a predisposition for neoplastic transformation of a lesion or polyp in the subject.

The present invention provides reagents for performing the diagnostic/predictive tests, in particular an isolated nucleic acid probe or primer that is capable of selectively hybridising to a region of the MCC gene promoter that is hyper methylated in a colorectal cancer, wherein said region comprises or is contained within nucleotide residues from about position 284 to about position 403 or about position 404 of SEQ ID NO: 3 or SEQ ID NO: 17 or SEQ ID NO: 24.

### BRIEF DESCRIPTION OF THE DRAWINGS

FIG. 1 is a copy of a schematic representation showing the double-stranded nucleotide sequence of the MCC gene promoter and the positions of CpG islands therein. CpG islands in the promoter are indicated by the shaded boxes that occur within the bracketed region to the left of the figure. The translation initiation site in the MCC gene is also indicated by the shaded ATG and the arrow.

FIG. 2 is a copy of a schematic representation showing the double-stranded nucleotide sequence of the MCC gene promoter from a variety of colon cancer cells following treatment with bisulphite. CpG islands in the promoter that are protected from mutagenesis by bisulphite are indicated by the shaded boxes. Filled circles indicate cytosine residues within CpG islands for which methylation was observed in a colorectal cancer cell line, wherein the number of circles indicate the number of colorectal cancer cell lines which exhibit methylation at the indicated. position. The positions of the region between positions −542 to −392 relative to the start of translation, showing variable levels of CpG methylation in different cell lines, are indicated at the left of the figure. The position of a region of the MCC gene promoter, from position −304 to position −139, that is hyper methylated in 6/13 colon cancer cell lines tested, is also indicated at the left of the figure. The boxed nucleotide sequences indicate positions for annealing of sequencing primers (light boxes) and the position of a section of DNA from position −354 to position −320 that is resistant to bisulphite treatment albeit not necessarily as a consequence of methylation (heavy boxed sequence).

FIG. 3 is a copy of a photographic representation showing reduced expression of MCC as determined by RT-PCR in colon cancer cells HCT15 and LS411N compared to the ovarian cancer cell line TOV21G. RT-PCR was performed to amplify MCC-encoding mRNA from the cell lines indicated at the top of each lane. The panel to the right of the figure is a control showing ALAS-1 expression in all three cell lines. Data show that expression of MCC MRNA is reduced in the colon cancer cell lines HCT15 and LS411N, compared to the ovarian cancer cell line TOV21G, however expression of ALAS-1 is similar in all three cell lines. These data are correlated with methylation data for the cell lines HCT15 and LS411N which indicate that the region of the MCC promoter from position −304 to position −139 is hyper methylated in both cell lines (i.e., all 26 CpG islands in this region are methylated in the colon cancer cell lines). In contrast, there was variable methylation in the between positions −465 and −391 of the

MCC promoter: All ten CpG sites showed methylation in both cell lines, but in cell line LS411N all ten sites were a mixture of methylated and non-methylated cytosines, whereas in cell line HCT15, 2/10 CpG islands in this region of the promoter were partially methylated. Accordingly, the reduced expression of MCC in the colon cancer cell lines is correlated to the the hyper methylation in the region of the MCC promoter from position −304 to position −139.

FIG. 4 is a copy of a schematic representation showing the double-stranded nucleotide sequence of the MCC gene promoter and the positions of primers for methylation-specific PCR to detect methylated CpG islands therein. The double-stranded nucleotide sequence of the MCC gene promoter from a variety of colon cancer cells following treatment with bisulphite is indicated, showing the positions of CpG islands in the promoter that are protected from mutagenesis by bisulphite (shaded boxes) and the degree of methylation (filled circles) as for the legend to FIG. 2. The sequences of forward and reverse primers (heavy shaded sequences) for amplification of a methylated region of the MCC gene promoter that is diagnostic for colon cancer are also indicated. The arrows indicate the direction of extension from the primers furing amplification such that the intervening sequence between the primers is amplified. These primers only amplify MCC promoter sequences from cell lines in which the CpG islands within the priming sites are methylated and, as a consequence, protected from mutagenesis by bisulphite. Amplified fragments can be sequenced to confirm methylation.

FIG. 5 is a copy of a photographic representation showing the products of a methylation-specific PCR assay in several different cell lines as indicated at the top of each lane. Methylation-specific PCR was performed as described using the primers indicated in FIG. 4. The cell lines designated Lisp1, Co-115, LS513, KM12SM, LS411N, HCT15 and SW480 are colorectal cancer cell lines. Data indicated that methylation-specific PCR correlates with bisulphite sequencing results.

FIG. 6 is a copy of a photographic representation showing methylation of the MCC gene promoter in primary colorectal tumors (C), normal tissue (N) and cancer of the lymph node (CLN). Numbers at the top of colorectal tumor lanes refer to patients from whom the tumors were derived. The upper panels indicate results for methylation-specific PCR of the MCC gene promoter. Methylation of the MCC gene promoter was observed in primary cancer tissues from patient samples numbered 1, 2, 3, 4, 6, and 7 and also in the lymph node cancer from patient number 6. The lower panel indicates results wherein the MYOD gene, which is known to be independent of methylation, was amplified from the samples indicated at the top of the figure.

FIG. 7 is a graphical representation showing specific amplification of methylated MCC promoter using real-time PCR in a quantitative TaqMan assay format.

FIG. 8 is a graphical representation showing amplification of MYOD gene sequences following treatment with bisulphite using real-time PCR in a quantitative TaqMan assay format, used as a control for the integrity of DNA samples in assays performed to determine methylation of MCC promoter sequences.

FIG. 9 is a schematic representation showing methylation of MCC, p16 and APC gene promoters in seven representative hyperplastic polyps (HP1, HP2, HP3, HP4, HP5, HP6 and HP7) and twelve representative adenomas (AD1, AD2, AD3, AD4, AD5, AD6, AD7, AD8, AD9, AD10, AD11 and AD12). The shading indicates methylation as determined using methylation specific PCR.

### DETAILED DESCRIPTION OF THE PREFERRED EMBODIMENTS

Diagnostic Assay Formats

I. Detection of Methylation within the MCC Gene

One aspect of the present invention provides a method of diagnosing colorectal cancer in a subject, said method comprising determining the degree of methylation of nucleic acid that regulates expression of an MCC gene in a sample derived from the subject, wherein an enhanced degree of methylation in the sample relative to a suitable control sample is indicative of colorectal cancer.

As used herein, the term “diagnosis”, and variants thereof, such as, but not limited to “diagnose”, “diagnosed” or “diagnosing” shall not be limited to a primary diagnosis of a clinical state, however should be taken to include any primary diagnosis of a clinical state. For example, the “diagnostic assay” formats described herein are equally relevant to assessing the remission of a patient, or monitoring disease recurrence, or tumor recurrence, such as following surgery, radiation therapy, adjuvant therapy or chemotherapy, or determining the appearance of metastases of a primary tumor. All such uses of the assays described herein are encompassed by the present invention.

In the present context, the term “colorectal cancer” shall be taken to include a chromosomal abnormality in a cell of the colon and/or rectum of a human or non-human mammal characterized by deletion, reduced expression or silencing of the region linked to map position 5q21-22 of the human genome or corresponding position in the genome of a non-human mammal, or a condition selected from the group consisting of Stage 0 (carcinoma in situ) especially characterized by the presence of epithelian lesions such as serrated lesions (e.g., ACF, traditional HP, variant HP, MP, SA or SSA), stage A colorectal cancer, stage B colorectal cancer, stage C colorectal cancer, stage D coldrectal cancer, or hyperplastic polyposis or a tumor arising therefrom including carcinoma, adenocarcinoma, sarcoma, lymphoma, carcinoid or gastrointestinal stromal tumor.

Those skilled in the art will be aware that as a carcinoma progresses, metastases occur in organs and tissues outside the site of the primary tumor. For example, in the case of colorectal cancer, metastases commonly appear in a tissue selected from the group consisting of lymph nodes, lung, liver, and bone. Accordingly, the term “colorectal cancer” as used herein shall be taken to include an early or developed tumor of the colon and/or rectum, such as, for example, any metastases thereof.

As used herein, the term “MCC gene” shall be taken to mean a nucleic acid, including any genomic gene, that is linked to or positioned at map position 5q21-q22 of the human genome, or any MRNA transcript thereof, or any genomic gene or MRNA transcript from a human or non-human animal that comprises a nucleotide sequence having at least about 80% identity to the sequence of a human MCC gene transcript as set forth in SEQ ID NO: 1. As will be known to those skilled in the art, mutations within the 5q21-22 region of the human genome are common in colorectal cancer and, as a consequence, the term “MCC gene” clearly encompasses any mutation of a wild-type or naturally-occurring MCC gene, including any nucleic acid deletion thereto or any nucleic acid substitution or insertion therein. The term “MCC gene” shall also be taken to include a gene encoding a peptide, polypeptide or protein from a human or non-human animal having an amino acid sequence that is at least about 80% identical to the sequence set forth in SEQ ID NO: 2.

For the purposes of nomenclature, the nucleotide sequence set forth in SEQ ED NO: 1 relates to the mRNA transcript of the human MCC gene (Kinder et al., Science 251, 1366-1370, 1991).

Preferably, the percentage identity to SEQ ID NO: 1 or 2 is at least about 85%, more preferably at least about 90%, even more preferably at least about 95% and still more preferably at least about 99%. In a particularly preferred embodiment, the MCC gene is a human MCC gene.

In determining whether or not two nucleotide sequences fall within these defined percentage identity limits, those skilled in the art will be aware that it is necessary to conduct a side-by-side comparison of sequences. In such comparisons or alignments, differences will arise in the positioning of non-identical nucleotide residues depending upon the algorithm used to perform the alignment. In the present context, references to percentage identities between two or more nucleotide sequences shall be taken to refer to the number of identical residues between said sequences as determined using any standard algorithm known to those skilled in the art. In particular, nucleotide identities and similarities are calculated using the BLAST or BESTFIT programs of the Computer Genetics Group, Inc., University Research Park, Madison, Wis., United States of America, to maximize the number of identical/similar amino acids and to minimize the number and/or length of sequence gaps in the alignment

The term “MCC gene” shall also be taken to include any variant of SEQ ID NO: 1, such as, for example, a variant that encodes a variant of the amino acid sequence set forth in SEQ ID NO: 2 wherein arginine at position 506 is substituted for glutamine (i.e., MCC R506Q) and/or wherein lysine at position 233 is substituted for threonine (i.e., MCC K233T) and/or wherein glutamate at position 234 is substituted for alanine (i.e., MCC E234A).

The term “nucleic acid that regulates expression of an MCC gene” shall be taken to mean a nucleic acid operably linked to the coding region of said gene in a human or animal subject wherein said nucleic acid confers, promotes or otherwise modulates expression of said gene in a cell of the subject, including any enhancer element, silencer element, or promoter, preferably contained within the 5-untranslated region of the MCC gene. In view of the diagnostic applications of the present invention, it is to be understood that the term “acid that regulates expression of an MCC gene” does not extend to a heterologous promoter that is used to regulate expression of an MCC gene in a cell that has been transformed or transfected with a recombinant vector comprising the heterologous promoter operably linked to the MCC gene.

Reference herein to a “promoter” is to be taken in its broadest context and includes the transcriptional regulatory sequences of a classical genomic gene, including the TATA box which is required for accurate transcription initiation, with or without a CCAAT box sequence and additional regulatory elements (i.e., upstream activating sequences, enhancers and silencers) which alter gene expression in response to developmental and/or external stimuli, or in a tissue-specific manner. In the present context, the term “promoter” is also used to describe a regulatory sequence that confers, activates or enhances the expression of an MCC gene to which it is operably connected and which encodes an MCC polypeptide in vivo. By “operable connection” is meant that the promoter is positioned such that it regulates expression of the MCC gene, generally 5′ (upstream) relative to the coding sequence and positioned within 2 kb of the start site of transcription of the gene.

For the purposes of nomenclature, the 5-untranslated region of a human MCC gene (i.e. SEQ ID NO: 1) is set forth as SEQ ID NO: 3. In this respect, the first 220 nucleotides of transcribed sequence in SEQ ID NO: 1 correspond to the 3′-terminal 220 residues of SEQ ID NO: 3.

Preferably, the nucleic acid that regulates expression of an MCC gene comprises the nucleotide sequence set forth in SEQ ID NO: 3, more preferably, nucleotides from about position 1 to about position 560 of SEQ ID NO: 3 and still more preferably nucleotides from about position 280 to about position 404 of SEQ ID NO: 3. In this context, the term “about” shall be taken to include at least 1-10 nucleotide residues upstream or downstream of the stated residue limitations. As exemplified herein, hyper methylation of several CpG islands in the 5′-terminal 560 residues of SEQ ID NO: 3 is diagnostic of colorectal cancer, with hyper methylation of a region from position 292 to position 458 has a higher correlation with carcinoma in situ especially the occurrence of hyperplastic polyps, or stage C colorectal cancer. Accordingly, the detection of hyper methylated residues in an amplified nucleic acid fragment comprising these nucleotides, in particular an amplified fragment consisting of residues 284 to 404, has diagnostic utility in the methods described herein.

In an even more particularly preferred embodiment, the detection of hyper methylated residues in a segment selected from the group consisting of:
(i) a segment comprising residues from position 284 to position 304; and/or
(ii) a segment comprising residues from position 335 to position 355; and/or
(iii) a segment comprising residues from position 361 to position 404, is diagnostic of colorectal cancer in a human subject.

The term “methylation of nucleic acid” shall be taken to mean the addition of a methyl group by the action of a DNA methyl transferase enzyme to a “CpG island” of nucleic acid, eg., genomic DNA. Without being bound by any theory or mode of action, hyper methylation of a CpG island results in a “closed” conformation that reduces or silences gene expression. As described in detail herein, there are several methods known to those skilled in the art for determining the level or degree of methylation, including hypo methylation or hyper methylation of nucleic acid.

By “enhanced” is meant that there are a significantly larger number of methylated CpG islands in the subject diagnosed than in the reference or suitable control sample. The present invention is not to be limited by a precise number of methylated residues that are considered to be diagnostic of colorectal cancer-in a subject, because some variation between patient samples will occur depending upon the tumor stage, positioning of the methylated residue and other global effects such as, for example, a secondary structure of nucleic acid that may hinder detection of methylated residues. For example, notwithstanding extensive methylation in the 5-untranslated region of a human MCC gene (i.e. SEQ ID NO: 1) in cancer cells lines derived from hyperplastic polyps or Stage C patients, several CpG sites may be protected directly or indirectly from methylation by secondary structure, or variably methylated between samples. Moreover, notwithstanding the significant utility of methylation within residues 284 to 404 of SEQ ID NO: 3 as a diagnostic, not all subjects having colorectal cancer will necessarily exhibit methylation of all residues in the region consisting of residues 284 to 404 of SEQ ID NO: 3. Preferably, at least about 3 or 4 or 5 or 6 or 7 or 8 or 9 or 10 or 11 or 12 or 13 or 14 or 15 or 16 or 17 or 18 CpG islands are methylated in a subject having hyperplastic polyps, serrated adenoma, mixed polyps, hyperplastic polyposis or more advanced colorectal cancer.

It is also to be understood that the methods described herein are equally applicable to the detection of colorectal cancer at any stage of the disease, including a stage as early as the transition from Stage 0 (i.e., polyposis) to Stage I (i.e., colorectal tumor localized to the colon lining) or Stage II (i.e., tumor that has penetrated the wall of the colon into the abdominal cavity but not elsewhere). The detection of colorectal cancer at a later stage of the disease is also encompassed by the present invention, such as, for example, the detection of Stage III or Stage IV colorectal cancers.

Such enhanced methylation will preferably be apparent in at least about 10-20% of subjects having Stage 0 or Stage A or Stage B or Stage C colorectal cancer or colorectal cancer at a later stage of the disease. More preferably, enhanced methylation will preferably be apparent in at least about 20-30% of subjects having colorectal cancer at a stage of carcinoma in situ (especially the occurrence of hyperplastic polyps, serrated polyps or mixed polyps) to Stage C. More preferably, enhanced methylation will preferably be apparent in at least about 30-40% or 40-50% or more of subjects having colorectal cancer at a stage of carcinoma in situ to Stage C. In a particularly preferred embodiment, the enhanced methylation of MCC promoter region is detected in at least about 80% to about 100% of hyperplastic polyps (HP) and at least about 50% of Stage C colorectal tumors, especially HP and tumors from the proximal colon. However, it is to be understood that, consistent with the lack of localization of variant HP, the present invention is useful for detecting a polyp or tumor (e.g., HP, SA, MP, or Stage C tumor etc.), elsewhere in the colorectum e.g., the splenic flexure and/or distal colon.

Similarly, by employing the methods described herein, the present invention is also useful for detecting and/or diagnosing a cancer/tumor selected from the group consisting of carcinoma, adenocarcinoma, sarcoma, lymphoma, carcinoid stromal tumor and gastrointestinal stromal tumor in a subject at any stage of the tumor.

Standard means known to the skilled artisan may be used to determine the level of methylation in the subject's sample and the control sample. The methods available for assessing DNA methylation status have varying degrees of sensitivity.

a) Methylation-sensitive Endonuclease Digestion of DNA

In one embodiment, the enhanced methylation in a subject sample is determined using a process comprising treating the nucleic acid with an amount of a methylation-sensitive restriction endonuclease enzyme under conditions sufficient for nucleic acid to be digested and then detecting the fragments produced. Exemplary methylation-sensitive endonucleases include HpaI and HpaII.

Preferably, assays include internal controls that are digested with a methylation-insensitive enzyme having the same specificity as the methylatior-sensitive enzyme employed. For example, the methylation-insensitive enzyme MspI is an isoschizomer of the methylation-sensitive enzyme HpaII.

Hybridization Assay Formats

Suitable detection methods for achieving selective hybridisation to the hybridisation probe include, for example, Southern or other nucleic acid hybridization (Kawai et al., Mol. Cell. Biol. 14, 7421-7427, 1994; Gonzalgo et al., Cancer Res. 57, 594-599, 1997).

The term “selectively hybridizable” means that the probe is used under conditions where a target nucleic acid, e.g., the MCC gene promoter, hybridizes to the probe to produce a signal that is significantly above background (i.e., a high signal-to-noise ratio). The intensity of hybridization is measured, for example, by radiolabeling the probe, e.g. by incorporating [α-³⁵S] and/or [α-³²P]dNTPs, [γ-³²P]ATP, biotin, a dye ligand (e.g., FAM or TAMRA), fluorophore, or other suitable ligand into the probe prior to use and then detecting the ligand following hybridization.

Suitable hybridization conditions are determined based on the melting temperature (Tm) of a nucleic acid duplex comprising the probe, e.g., as described by Berger and Kimmel (1987, Guide to Molecular Cloning Techniques, Methods in Enzymology, Vol 152, Academic Press, San Diego Calif.).

The skilled artisan will be aware that optimum hybridisation reaction conditions should be determined empirically for each probe, although some generalities can be applied. Preferably, hybridisations employing short oligonucleotide probes are performed at low to medium stringency. For the purposes of defining the level of stringency, a low-stringency hybridization is achieved using a hybridization buffer and/or a wash solution comprising the following:
(i) a salt concentration that is equivalent to about 2×SSC to about 6×SSC buffer; SDS; and
(iii) an incubation temperature between about room temperature and about 45° C.

In this respect, the skilled artisan is aware that 1×SSC consists of 0.15 M NaCl, 0.015 M Na₃ Citrate pH 7.0.

Moderate stringency hybridisation conditions will generally be achieved using a hybridization buffer and/or a wash solution comprising:
(i) a salt concentration that is equivalent to about 0.1×SSC to about 2×SSC buffer;
(ii) a detergent concentration equivalent to 0.1% (w/v) SDS to about 1% (w/v) SDS; and
(iii) an incubation temperature between about 42° C. and about 55° C.

For long probes or oligonucleotides having high G+C content (high melting temperature), high stringency hybridisation conditions can achieve a high signal-to-noise ratio as desired. High stringency hybridisation conditions will generally be achieved using a hybridization buffer and/or a wash solution comprising:
(i) a salt concentration that is equivalent to about 0.1×SSC buffer or lower salt concentration;
(ii) a detergent concentration equivalent to 0.1% (w/v) SDS or lower detergent concentration; and
(iii) an incubation temperature above about 55° C. and preferably above about 65° C.

Conditions for specifically hybridizing nucleic acid, and conditions for washing to remove non-specific hybridizing nucleic acid, are well understood by those skilled in the art. For the purposes of further clarification only, reference to the parameters affecting hybridization between nucleic acid molecules is found in Ausubel et al. (Current Protocols in Molecular Biology, Wiley Interscience, ISBN 047150338, 1992), which is herein incorporated by reference.

For detecting fragments produced by endonuclease digestion using a hybridisation assay format, any suitable hybridisation probe derived from the promoter region of the MCC gene set forth in SEQ ID NO: 3 can be used in accordance with standard procedures. This is because the detection involves hybridisation to all fragments produced, as opposed to a selective hybridization, and then comparing the fragments produced in the test sample to those fragments produced for a suitable control sample.

Preferred hybridization probes will comprise at least about 18 contiguous nucleotides in length from SEQ ID NO: 3, more preferably at least about 50 contiguous nucleotides from SEQ ID NO: 3, preferably incorporating one or more CpG islands that are hyper methylated in colorectal cancer. Preferred probes will hybridise to a region that includes residues 284-404 of SEQ ID NO: 3 or a sequence that is complementary thereto, or a portion thereof including one or more CpG islands that are hyper methylated in colorectal cancer.

In a particularly preferred embodiment, the probe will include the MspI/HpaII recognition site located at position 291 to position 294 of SEQ ID NO: 3. In this respect, digestion of nucleic acid from a colorectal cancer sample with HpaII will fail to cleave at this position, thereby producing a larger DNA fragment than for a healthy/normal control sample in which the site is not methylated. In contrast, the methylation-insensitive enzyme MspI will produce the same-size fragment for both the colorectal cancer sample and the control sample.

As will be known to the skilled artisan, longer probes are preferred, because these generally produce higher signal-to-noise ratio than shorter probes and/or permit higher stringency hybridisation and wash conditions to be employed. Accordingly, it is preferable to use hybridization probes that comprise at least about 100 contiguous nucleotides from SEQ ID NO: 3 and even more preferably at least about 200 contiguous nucleotide residues. As will be known to the skilled artisan, restriction fragments derived from the MCC gene promoter are particularly useful for such applications.

In accordance with this embodiment, a difference in the fragments produced for the test sample and a negative control sample is indicative of the subject having colorectal cancer. Similarly, in cases where the control sample comprises data from a tumor, cancer tissue or a cancerous cell or pre-cancerous cell, similarity, albeit not necessarily absolute identity, between the test sample and the control sample is indicative of a positive diagnosis (i.e. colorectal cancer).

Amplification Assay Formats

Methylation-sensitive endonuclease-based assays are straightforward, however are generally of low sensitivity and require large amounts of input DNA, unless linked to a PCR detection method. Such linkage is clearly encompassed by the present invention.

Accordingly, in an alternative embodiment, the fragments produced by the restriction enzyme are detected using an amplification system, such as, for example, polymerase chain reaction (PCR), rolling circle amplification (RCA), inverse polymerase chain reaction (iPCR), in situ PCR (Singer-Sam et al., Nucl. Acids Res. 18, 687,1990), strand displacement amplification (SDA) or cycling probe technology.

Methods of PCR are known in the art and described, for example, by McPherson et al., PCR: A Practical Approach. (series eds, D. Rickwood and B. D. Hames), IRL Press Limited, Oxford. pp1-253, 1991 and by Dieffenbach (ed) and Dveksler (ed) (In: PCR Primer: A Laboratory Manual, Cold Spring Harbour Laboratories, NY, 1995), the contents of which are each incorporated in their entirety by way of reference. Generally, for PCR two non-complementary nucleic acid primer molecules comprising at least about 18 nucleotides in length, and more preferably at least 20-30 nucleotides in length are hybridised to different strands of a nucleic acid template molecule at their respective annealing sites, and specific nucleic acid molecule copies of the template that intervene the annealing sites are amplified enzymatically. Amplification products may be detected using electrophoresis and detection with a detectable marker that binds nucleic acids. Alternatively, one or more of the oligonucleotides are labelled with a detectable marker (e.g. a fluorophore) and the amplification product detected using, for example, a lightcycler (Perkin Elmer, Wellesley, Mass., USA).

Strand displacement amplification (SDA) utilises oligonucleotide primers, a DNA polymerase and a restriction endonuclease to amplify a target sequence. The oligonucleotides are hybridized to a target nucleic acid and the polymerase is used to produce a copy of the region intervening the primer annealing sites. The duplexes of copied nucleic acid and target nucleic acid are then nicked with an endonuclease that specifically recognises a sequence at the beginning of the copied nucleic acid. The DNA polymerase recognises the nicked DNA and produces another copy of the target region at the same time displacing the previously generated nucleic acid. The advantage of SDA is that it occurs in an isothermal format, thereby facilitating high-throughput automated analysis.

Cycling Probe Technology uses a chimeric synthetic primer that comprises DNA-RNA-DNA that is capable of hybridising to a target sequence. Upon hybridisation to a target sequence the RNA-DNA duplex formed is a target for RNaseH thereby cleaving the primer. The cleaved primer is then detected.

Preferred amplification primers will comprise at least about 18 contiguous nucleotides in length from SEQ ID NO: 3, more preferably at least about 50 contiguous nucleotides from SEQ ID NO: 3, preferably incorporating one or more CpG islands that are hyper methylated in colorectal cancer and flanking or comprising a methylation-sensitive endonuclease recognition site.

For primers that flank a methylation-sensitive endonuclease recognition site, it is preferred that such primers flank only those sites that are hyper methylated in colorectal cancer to ensure that a diagnostic amplification product is produced.

Preferred primers will flank a region that includes residues 284-404 of SEQ ID NO: 3 or a sequence that is complementary thereto, or a portion thereof including one or more CpG islands that are hyper methylated in colorectal cancer.

In a more preferred embodiment, the amplification primers are selected such that they flank the MspI/HpaII recognition site located at position 291 to position 294 of SEQ ID NO: 3. In this respect, amplified nucleic acid from a healthy control, but not from a colorectal cancer sample will cleave at this position following digestion with HpaII. Amplification products using such primers are expected for both a colorectal cancer sample and a normal/healthy control, however a larger amplification product is produced for a colorectal cancer sample than for a control sample as a consequence of cleavage at the HpaII recognition site by the enzyme HpaII. In a negative control amplification reaction employing MspI, the shorter amplification product is produced for both a colorectal cancer sample and a normal/healthy control using such primers.

As will be known to the skilled artisan, the precise length of the amplified product will vary depending upon the distance between the primers.

In a particularly preferred embodiment, at least one amplification primer will comprise the MspI/HpaII recognition site located at position 291 to position 294 of SEQ ID NO: 3 or a sequence that is capable of hybridising to said recognition site or the complement thereof. Accordingly, a primer that comprises this recognition sequence will produce an amplification product only when the site is methylated. In contrast, the methylation-insensitive enzyme MspI will produce the same-size fragment for both the colorectal cancer sample and the control sample. Preferably, the primer includes the HpaII recognition sequence at its 3′-end such that amplification of downstream nucleic acid occurs when the HpaII recognition sequence has not been cleaved (i.e., in the colorectal cancer sample), and not when cleaved (i.e., in a healthy or normal control sample) by HpaII. In a negative control amplification reaction employing MspI, no amplification product is produced using such a primer.

In these methods, one or more of the primers may be labelled with a detectable marker to facilitate rapid detection of amplified nucleic acid, for example, a fluorescent label (e.g. Cy5 or Cy3) or a radioisotope (e.g. ³²p)

The amplified nucleic acids are generally analysed using, for example, non-denaturing polyacrylamide gel electrophoresis, mass spectrometry (e.g., MALDI-TOF, Sequenome), liquid chromatography (eg. HPLC or dHPLC), or capillary electrophoresis. High throughput detection methods, such as, for example, matrix-assisted laser desorption/ionization time of flight (MALDI-TOF), electrospray ionization (ESI), Mass spectrometry (including tandem mass spectrometry, eg LC MS/MS), biosensor technology, evanescent fiber-optics technology or DNA chip technology (e.g., WO98/49557; WO 96/17958; Fodor et al., Science 767-773, 1991; U.S. Pat. No. 5,143,854; and U.S. Pat. No. 5,837,832, the contents of which are all incorporated herein by reference), are especially preferred for all assay formats described herein, especially when screening of large numbers of samples, such as, for example, public health screening of subjects having a higher risk of developing colorectal cancer. A particular example of a suitable solid substrate for producing DNA chip arrays is the commercially available BIACore™ chip (Pharmacia Biosensors).

Alternatively, amplification of a nucleic acid may be continuously monitored using a melting curve analysis method, such as that described in, for example, U.S. Pat. No. 6,174,670, which is incorporated herein by reference.

Alternatively, or in addition, the nucleotide sequence of the amplified DNA is determined according to standard procedures.

(b) SSCP Based Assays

In an alternative embodiment of the present invention, the enhanced methylation in a subject sample is determined by performing a process comprising treating the nucleic acid with an amount of DNaseI under conditions sufficient for nucleic acid to be digested and then detecting the fragments produced.

This assay format is predicated on the understanding that methylated DNA has a more tightly-closed conformation than non-hyper methylated DNA and, as a consequence, is less susceptible to endonuclease digestion by DNase I.

In accordance with this embodiment, DNA fragments of different lengths are produced by DNase I digestion of methylated compared to non-hyper methylated DNA.

Hybridization Assay Format

To detect the fragments of differing length produced by DNase I digestion, art-recognized detection methods are employed, such as, for example, Southern or other nucleic acid hybridization (Kawai et al., Mol. Cell. Biol. 14, 7421-7427, 1994; Gonzalgo et al., Cancer Res. 57, 594-599, 1997). Such assay formats are generally described herein above and apply mutatis mutandis to SSCP-based processes presently described.

As with the use of methylation-sensitive endonucleases in a hybridisation assay format, the probe can be selected from any portion of SEQ ID NO: 3 that incorporates or flanks a region that is hyper methylated in colorectal cancer. Preferred probes will hybridise to a region that includes residues 284-404 of SEQ ID NO: 3 or a sequence that is complementary thereto, or a portion thereof.

For Southern hybridization detection systems, or non-PCR detection systems, a difference in the length of fragments produced for the test sample and a negative control sample is indicative of the subject having colorectal cancer. Preferably, a larger fragment is produced from the colorectal cancer patient sample, Similarly, in cases where the control sample comprises data from a tumor, cancer tissue or a cancerous cell or pre-cancerous cell, similarity, albeit not necessarily absolute identity, between the test sample and the control sample is indicative of a positive diagnosis (i.e. colorectal cancer).

High throughput detection methods, such as, for example, matrix-assisted laser desorption/ionization time of flight (MALDI-TOF), electrospray ionization (ESI), Mass spectrometry (including tandem mass spectrometry, eg LC MS/MS), biosensor technology, evanescent fiber-optics technology or DNA chip technology, can also be employed.

Amplification Assay Format

In an alternative embodiment, an amplification assay is used to detect the fragments of differing length produced by DNase I digestion, such as, for example, polymerase chain reaction (PCR), rolling circle amplification (RCA), inverse polymerase chain reaction (iPCR), in situ PCR (Singer-Sam et al., Nucl. Acids Res. 18, 687,1990), strand displacement amplification, or cycling probe technology. Subject to appropriate selection of probes and/or primers as described below, such assay formats are generally described herein above and apply mutatis mutandis to SSCP-based processes presently described.

PCR-SSCP is performed essentially as described by Gregory and Feil Nucleic Acids Res., 27, e32i-e32iv, 1999. High throughput detection methods, such as, for example, matrix-assisted laser desorption/ionization time of flight (MALDI-TOF), electrospray ionization (ESI), Mass spectrometry (including tandem mass spectrometry, eg LC MS/MS), biosensor technology, evanescent fiber-optics technology or DNA chip technology, can also be employed.

In general, the assay format comprises a positive read-out system in which DNA from a colorectal cancer sample is detected as a positive signal. In one embodiment, nucleic acid is subjected to DNase I digestion and the fragments produced are amplified using primers that bind to or flank methylated regions of the MCC gene promoter in a polymerase chain reaction or equivalent detection means.

For PCR-SSCP, amplification primers flanking or comprising one or more CpG islands that are resistant to DNase I digestion in a colorectal cancer sample but not resistant to DNase I digestion in a healthy/normal control or healthy/normal test sample are used to amplify the DNase I-generated fragments. In this case, the production of a specific nucleic acid fragment using DNase I is diagnostic of colorectal cancer, because the DNA is not efficiently degraded. In contrast, template DNA from a healthy/normal subject sample is degraded by the action of DNase I and, as a consequence, amplification fails to produce a discrete amplification product.

Primers suitable for such an assay format are readily derived from the description herein of hyper methylated CpG islands in the 5-terminal 560 residues of SEQ ID NO: 3. In a preferred embodiment, the primers are selected from the group consisting of (i) a primer comprising a sequence contained within residues from position 284 to position 304 of SEQ ID NO: 3; (ii) a primer comprising a sequence contained within residues from position 335 to position 355 of SEQ ID NO: 3; (iii) a primer comprising a sequence contained within residues from position 361 to position 382 of SEQ ID NO: 3; (iv) a primer comprising a sequence contained within residues from position 383 to position 404 of SEQ ID NO: 3; and (v) a primer comprising a sequence that is complementary to any one of (i) to (iv).

Even more preferably, each primer will comprise a sequence as set forth in SEQ ID Nos: 11-16 or complementary to any one or more of SEQ ID Nos: 11-16. Minor sequence variants thereof that have substantially the same melting temperature as the exemplified probes may also be used.

For the purposes of nomenclature, the nucleotide sequence set forth in SEQ ID NO: 11 corresponds to residues at positions 284 to 304 of SEQ ID NO: 3 in the 5-untranslated region of the MCC gene promoter. The sequence set forth in SEQ ID NO: 12 corresponds to residues at positions 335 to 355 of SEQ ID NO: 3 in the 5-untranslated region of the MCC gene promoter. The sequence set forth in SEQ ID NO: 13 corresponds to the complement of residues at positions 361 to 382 of SEQ ID NO: 3 in the 5-untranslated region of the MCC gene promoter. The sequence set forth in SEQ ID NO: 14 corresponds to residues at positions 335 to 354 of SEQ ID NO: 3 in the 5-untranslated region of the MCC gene promoter. The sequence set forth in SEQ ID NO: 15 corresponds to residues at positions 359 to 377 of SEQ ID NO: 3 in the 5-untranslated region of the MCC gene promoter. The sequence set forth in SEQ ID NO: 16 corresponds to the complement of residues at positions 383 to 404 of SEQ ID NO: 3 in the 5-untranslated region of the MCC gene promoter.

Still more preferably, for any detection format described herein that comprises an amplification step, the primers used will be a combination selected from the group consisting of:
(i) a primer comprising the sequence set forth in SEQ ID NO: 11 and a primer comprising a sequence that is the complement of SEQ ID NO: 12;
(ii) a primer comprising the sequence set forth in SEQ ID NO: 11 and a primer comprising the sequence set forth in SEQ ID NO: 13;
(iii) a primer comprising the sequence set forth in SEQ ID NO: 11 and a primer comprising a sequence that is the complement of SEQ ID NO: 14;
(iv) a primer comprising the sequence set forth in SEQ ID NO: 11 and a primer comprising a sequence that is the complement of SEQ ID NO: 15;
(v) a primer comprising the sequence set forth in SEQ ID NO: 11 and a primer comprising the sequence set forth in SEQ ID NO: 16;
(vi) a primer comprising the sequence set forth in SEQ ID NO: 12 and a primer comprising the sequence set forth in SEQ ID NO: 13;
(vii) a primer comprising the sequence set forth in SEQ ID NO: 12 and a primer comprising a sequence that is the complement of SEQ ID NO: 15;
(viii) a primer comprising the sequence set forth in SEQ ID NO: 12 and a primer comprising the sequence set forth in SEQ ID NO: 16;
(ix) a primer comprising the sequence set forth in SEQ ID NO: 13 and a primer comprising the sequence set forth in SEQ ID NO: 14;
(x) a primer comprising the sequence set forth in SEQ ID NO: 14 and a primer comprising a sequence that is the complement of SEQ ID NO: 15;
(xi) a primer comprising the sequence set forth in SEQ ID NO: 14 and a primer comprising the sequence set forth in SEQ ID NO: 16; and
(xii) a primer comprising the sequence set forth in SEQ ID NO: 15 and a primer comprising the sequence set forth in SEQ ID NO: 16.

It is to be understood that the detection step of the assay formats described herein clearly encompass the use of multiple rounds of amplifications and/or combinations of amplification and classical nucleic acid hybridization steps, in any order. For example, it is possible to amplify nucleic acid of the MCC gene promoter using a combination of a primer comprising the sequence set forth in SEQ ID NO: 11 and a primer comprising the sequence of SEQ ID NO: 13, and then to hybridize the amplified nucleic acid using a labeled hybridization probe that comprises the nucleotide sequence of SEQ ID NO: 12 or a complementary sequence thereto. Similarly, it is possible to amplify nucleic acid of the MCC gene promoter using a combination of a primer comprising the sequence set forth in SEQ ID NO: 14 and a primer comprising the sequence of SEQ ID NO: 16, and then to hybridize the amplified nucleic acid using a labeled hybridization probe that comprises the nucleotide sequence of SEQ ID NO: 15 or a complementary sequence thereto. The use of other hybridization probes capable of hybridizing to the amplified nucleic acid is clearly contemplated by the present invention, the only requirement being that the probe comprises a nucleotide sequence that occurs within the MCC gene promoter at a position between the two amplification primer sequences and preferably does not comprise a sequence of either amplification primer used. The skilled artisan will readily be capable of determining the nucleotide sequence of a suitable hybridization probe to perform this embodiment based upon the disclosure in SEQ ID NO: 3 and, as a consequence, the present invention is not to be limited by the precise sequence of a hybridization probe used in conjunction with amplification primers as described.

For multiple rounds of amplification, it is particularly preferred to use a nested amplification format to achieve enhanced specificity of the amplification products. For example, a first series of amplification reactions may employ a primer comprising the sequence set forth in SEQ ID NO: 11 and a primer comprising the sequence of SEQ ID NO: 13, and a second series of amplification reactions may employ: (i) a primer comprising the sequence set forth in SEQ ID NO: 11 and a primer comprising a sequence that is complementary to the sequence of SEQ ID NO: 12; or alternatively, (ii) a primer comprising the sequence set forth in SEQ ID NO: 12 and a primer comprising the sequence of SEQ ID NO: 13; or alternatively, (iii) a primer comprising the sequence set forth in SEQ ID NO: 12 and a primer comprising the sequence of SEQ ID NO: 14; or alternatively, (iv) a primer comprising the sequence of SEQ ID NO: 12 and a primer comprising the a sequence that is complementary to SEQ ID NO: 15; or alternatively, (v) a primer comprising the sequence of SEQ ID NO: 14 and a primer comprising the a sequence that is complementary to SEQ ID NO: 15. Similarly, for a first series of amplification reactions employing a primer comprising the sequence set forth in SEQ ID NO: 11 and a primer comprising the sequence of SEQ ID NO: 16, a second series of amplification reactions may employ anyone or more of the sequences set forth in SEQ ID Nos: 12-15 in an appropriate combination as described supra to amplify the intervening region there between. The performance of each and every of the above-mentioned second series of amplification reactions simultaneously or contemporaneously is also encompassed by the present invention.

Other primer combinations are also not to be excluded when using multiple amplifications to detect nucleic acid, the only requirement being that the primers are selected such that they comprise nucleotide sequences that occur within the MCC gene promoter at a position between the two amplification primer sequences used for the first series of amplifications. The skilled artisan will readily be capable of determining the nucleotide sequence of suitable amplification primers to perform this embodiment based upon the disclosure in SEQ ID NO: 3 and, as a consequence, the present invention is not to be limited by the precise sequence of second round amplification primers used in conjunction with the first round amplification primers as described. Combinations of the exemplified primers set forth in SEQ ID Nos: 11-16 are preferred for first and second round amplifications because these are derived from methylated sequences of the MCC gene promoter and, as a consequence, provide enhanced specificity in the detection of hyper methylated sequences.

The use of such methylated sequence-specific primers in all detection stages also provides for validation of a primary diagnosis of colorectal cancer based on the use of a single primer set or hybridization probe, and a reduced level of false positive diagnoses than would otherwise be the case.

In colorectal cancer subjects, methylation of cytosine residues within the CpG islands of these sequences in the MCC gene promoter is enhanced and, as a consequence, protected from DNase I digestion. This enables enhanced hybridization of one or more probes to the MCC gene promoter in the cancer sample thereby producing an enhanced signal relative to a normal/healthy control.

The present invention also encompasses the use of real-time quantitative forms of PCR, such as, for example, TaqMan (Holland et al., Proc. Natl Acad. Sci. USA, 88, 7276-7280, 1991; Lee et al., Nucleic Acid Res. 21, 3761-3766, 1993) to perform this embodiment. TaqMan indicates the probe used to detect specific sequences in amplified PCR products by employing the 5- to 3-exonuclease activity of Taq DNA polymerase. A TaqMan probe (about 20-30 nucleotides in length), disabled from extension at the 3-end, generally consists of a site-specific sequence labeled with a fluorescent reporter dye and a fluorescent quencher dye e.g., TAMRA or FAM, or alternatively or in addition, a Black Hole Quencher (BHQ) fluorescent dye that prevents fluorescence until a hybridization event occurs (Biosearch Technologies, Inc., Novato Calif. 94949-5750, USA). As will be known to the skilled artisan, each of the fluorescent reporter dye, fluorescent quencher dye and BHQ may be added to the 5-end or 3-end of the probe and the present invention encompasses all such alternatives. During PCR, the TaqMan probe hybridizes to its complementary single strand DNA sequence within the methylated region of the MCC gene promoter. When amplification occurs, the TaqMan probe is degraded due to the 5→3′ exonuclease activity of Taq DNA polymerase, thereby separating the quencher from the reporter during extension. Due to the release of the quenching effect on the reporter, the fluorescence intensity of the reporter dye increases. During the entire amplification process this light emission increases exponentially, the final level being measured by spectrophotometry upon termination of the PCR. Because increase of the fluorescence intensity of the reporter dye is only achieved when probe hybridization and amplification of the target sequence has occurred, the TaqMan assay offers a sensitive method to determine the presence or absence of specific sequences. The TaqMan assay allows high sample throughput because no gel-electrophoresis is required for detection.

In adapting the TaqMan assay to this positive read-out format, it is preferred to utilize a TaqMan probe comprising the sequences set forth in SEQ ID NOs: 12 or 15 labelled at its 5′- and 3′-ends with different fluorescent ligands, e.g., TAMRA and/or PAM and/or BHQ. In a particularly preferred embodiment, the probe is labelled at the 5′-end using FAM and BHQ and at the 3′-end using TAMRA, or alternatively, at the 5-end with TAMRA and BHQ and at the 3-end with PAM, or alternatively, at the 5′-end using FAM and at the 3′-end using TAMRA and BHQ, or alternatively, at the 5-end with TAMRA and at the 3-end with FAM and BHQ. Such a probe is generally used in conjunction with amplification primers having different and non-overlapping sequences with SEQ ID NOs: 12 or 15 in the MCC gene promoter, as described herein above.

In one preferred embodiment of the TaqMan assay, the amplification primers set forth in SEQ ID Nos: 11 and 13 are used in conjunction with a TaqMan probe comprising the nucleotide sequence of SEQ ID NO: 12. In an alternative embodiment, the amplification primers set forth in SEQ ID Nos: 14 and 16 are used in conjunction with a TaqMan probe comprising the nucleotide sequence of SEQ ID NO: 15.

c) Selective Mutagenesis of Non-hyper Methylated DNA

In an alternative embodiment of the present invention, the enhanced methylation in a subject sample is determined using a process comprising treating the nucleic acid with an amount of a compound that selectively mutates a non-methylated cytosine residue within a CpG island under conditions sufficient to induce mutagenesis.

Preferred compounds mutate cytosine to uracil or thymidine, such as, for example, a metal salt of bisulphite, eg., sodium bisulphite or potassium bisulphite (Frommer et al., Proc. Natl. Acad. Sci. USA 89, 1827-1831, 1992). Bisulfite treatment of DNA is known to distinguish methylated from non-methylated cytosine residues, by mutating cytosine residues that are not protected by methylation, including cytosine residues that are not within a CpG island or that are positioned within a CpG island that is not subject to methylation. Both strands of a sequence are susceptible to mutagenesis using bisulphite.

The nucleotide sequence of one strand of bisulphite treated DNA, containing the methylated region of the MCC gene promoter is set forth in SEQ ID NO: 17. For the purposes of nomenclature, the nucleotide sequence set forth in SEQ ID NO: 17 relates to the 5-untranslated region of a bisulphite-mutated MCC gene comprising cytosine residues that have been protected from mutagenesis by hyper methylation in colorectal cancer cells. In this respect, SEQ ID NO: 17 provides a mutated version of the wild-type sequence set forth in SEQ ID NO: 3.

The nucleotide sequence of a non-hyper methylated MCC gene promoter following bisulphite treatment is set forth in SEQ ID NO: 24. For the purposes of nomenclature, the nucleotide sequence set forth in SEQ ID NO: 24 relates to the 5-untranslated region of a bisulphite-mutated MCC gene wherein, due to the absence of methylation, diagnostic cytosine residues have not been protected from mutagenesis. In this respect, SEQ ID NO: 24 provides a mutated version of the wild-type sequence set forth in SEQ ID NO: 3 and differs from SEQ ID NO: 17 in so far as diagnostic CpG islands have not been protected from mutation.

The skilled artisan may readily determine nucleotide sequences of both the methylated and non-methylated forms of the MCC promoter, by performing bisulphite sequencing of the complementary strands in normal and colorectal cancer tissues. This generally involves determining the pattern of methylation on the opposing strand and/or the nucleotide sequences of the opposing strands to SEQ ID Nos: 3, 17 or 24 following bisulphite treatment. In this respect, the cytosine residues on both strands are converted to thymine residues by bisulphite unless protected by methylation.

Depending upon the assay format in question, suitable probes and primers for performing the following embodiments of the invention are readily derived from SEQ ID NO: 17 and/or SEQ ID NO: 24.

(c)(i) Positive Read-out Assay Format

In one embodiment, the assay format comprises a positive read-out system in which DNA from a colorectal cancer sample is detected as a positive signal. Preferably, the non-hyper methylated DNA from a healthy or normal control subject is not detected or only weakly detected.

In a preferred embodiment, the enhanced methylation in a subject sample is determined using a process comprising:
(i) treating the nucleic acid with an amount of a compound that selectively mutates a non-methylated cytosine residue within a CpG island under conditions sufficient to induce mutagenesis thereby producing a mutated nucleic acid;
(ii) hybridizing the nucleic acid to a probe or primer comprising a nucleotide sequence that is complementary to a sequence comprising the methylated cytosine residue under conditions such that selective hybridization to the non-mutated nucleic acid occurs; and
(iii) detecting the selective hybridization.

In this context, the term “selective hybridisation” means that hybridisation of a probe or primer to the non-mutated nucleic acid occurs at a higher frequency or rate, or has a higher maximum reaction velocity, than hybridisation of the same probe or primer to the corresponding mutated sequence. Preferably, the probe or primer does not hybridise to the non-hyper methylated sequence carrying the mutation(s) under the reaction conditions used.

For positive read-out assay formats that detect DNA from a colorectal cancer sample is detected as a positive signal following treatment with bisulphite, it is preferred to use probes and/or primers derived from SEQ ID NO: 17, in which cytosine residues from the wild-type MCC gene promoter have been mutated to thymidine other than those cytosine residues within a CpG island that is hyper methylated in a colorectal cancer sample.

Hybridization-based Assay Format

In one embodiment the hybridisation is detected using Southern, dot blot, slot blot or other nucleic acid hybridisation means (Kawai et al., Mol. Cell. Biol. 14, 7421-7427, 1994; Gonzalgo et al., Cancer Res. 57, 594-599, 1997). Subject to appropriate probe selection, such assay formats are generally described herein above and apply mutatis mutandis to the presently described selective mutagenesis approach.

Preferably, a ligase chain reaction format is employed to distinguish between a mutated and non-mutated MCC gene promoter. Ligase chain reaction (described in EP 320,308 and U.S. Pat. No. 4,883,750) uses at least two oligonucleotide probes that anneal to a target nucleic acid in such a way that they are juxtaposed on the target nucleic acid (i.e., MCC gene promoter sequence). In a ligase chain reaction assay, the target nucleic acid is hybridised to a first probe that is complementary to a diagnostic portion of the target sequence (the diagnostic probe) e.g., a methylated CpG island, and with a second probe that is complementary to a nucleotide sequence contiguous with the diagnostic portion (the contiguous probe), under conditions wherein the diagnostic probe remains bound substantially only to the target nucleic acid. The diagnostic and contiguous probes can be of different lengths and/or have different melting temperatures such that the stringency of the hybridization can be adjusted to permit their selective hybridisation to the target, wherein the probe having the higher melting temperature is hybridised at higher stringency and, following washing to remove unbound and/or non-selectively bound probe, the other probe having the lower melting temperature is hybridised at lower stringency. The diagnostic probe and contiguous probe are then covalently ligated such as, for example, using T4 DNA ligase, to thereby produce a larger target probe that is complementary to the target sequence, and the probes that are not ligated are removed by modifying the hybridisation stringency. In this respect, probes that have not been ligated will selectively hybridise under lower stringency hybridisation conditions than probes that have been ligated. Accordingly, the stringency of the hybridisation can be increased to a stringency that is at least as high as the stringency used to hybridise the longer probe, and preferably at a higher stringency due to the increased length contributed by the shorter probe following ligation.

It is preferred to melt the target-probe duplex, elute the dissociated probe and confirm that is has been ligated, e.g., by determining its length using electrophoresis, mass spectrometry, nucleotide sequence analysis, gel filtration, or other means known to the skilled artisan.

It will be apparent to the skilled artisan that, by virtue of the clustered arrangement of methylated cytosine residues within the MCC gene promoter as provided by the present invention, ligase chain reaction is particularly sensitive for distinguishing between multiple methylated residues and multiple non-hyper methylated residues, by careful selection of combinations of probes based upon the sequence set forth in SEQ ID NO: 17. Accordingly, in an alternative embodiment of the ligase chain reaction, the ligated probes are detected by adding an “anchor” primer following the ligation reaction and performing PCR in accordance with standard amplification procedures as described herein. The “anchor” primer is selected such that it comprises a nucleotide sequence that will anneal to the opposing strand to the ligated probe and have a 5′- to 3′-orientation that will permit amplification of target nucleic acid that intervenes the ligated probe and anchor primer. The length of the amplification product can then be confirmed in the usual manner wherein a longer product confirms the ligation event.

In another preferred mode, one or both of the probes is labeled such that the presence or absence of the target sequence can be tested by melting the target-probe duplex, eluting the dissociated probe, and testing for the label(s). Where both probes are labelled, different ligands are used to permit distinction between the ligated and unligated probes, in which case the presence of both labels in the same eluate fraction confirs the ligation event.

It is also known to attach a hook to a probe that is not labeled, so that the labeled probe may be recovered by catching the hook. In both instances, both the diagnostic probe and the contiguous probe are required to anneal to their respective target sites in the target nucleic acid for the label to appear in the assay.

If the target nucleic acid is bound to a solid matrix e.g., in a Southern hybridisation, slot blot, dot blot, or microchip assay format, the presence of both the diagnostic and contiguous probes can be determined directly.

Probes suitable for such an assay format are readily derived from the description herein of hyper methylated CpG islands in the 5-terminal 560 residues of SEQ ID NO: 17.

In accordance with this embodiment, the diagnostic probe and preferably also the contiguous probe should be selected such that they selectively hybridize to wild type sequences in the MCC gene promoter that are methylated in samples from subjects having colorectal cancer and thereby protected from mutation. By “selectively hybridize” in this context is meant that the probe(s) anneal at a significantly higher frequency under the conditions employed to a mutated target sequence of a hyper methylated MCC gene promoter derived from a colorectal cancer sample compared to a mutated target sequence of an MCC gene promoter derived from a healthy or normal control subject, thereby producing a high signal-to-noise ratio in the assay. Preferably, the probe(s) have 3′-terminal and/or 5′-terminal sequences that comprise a CpG island that is hyper methylated in colorectal cancer compared to a healthy or normal control subject, such that the diagnostic probe and contiguous probe are capable of being ligated only when the cytosine of the CpG island has not been mutated to thymidine e.g., in the case of a methylated cytosine residue.

In a preferred embodiment, the diagnostic and contiguous probes are selected from the group consisting of (i) a probe comprising a sequence contained within the sequence set forth in SEQ ID NO: 17 and having a 5′-terminal guanosine residue at a position within SEQ ID NO: 17 selected from the group consisting of position 293, position 298, position 303, position 339, position 342, position 348, position 360, position 362, position 365, position 367, position 373, position 395 and position 399; (ii) a probe comprising a sequence contained within the sequence set forth in SEQ ID NO: 17 and having a 3′-terminal cytosine residue at a position within SEQ ID NO: 17 selected from the group consisting of position 292, position 297, position 302, position 338, position 341, position 347, position 359, position 361, position 364, position 366 position 372, position 394 and position 398; (iii) a probe comprising a sequence that is complementary to a portion of SEQ ID NO: 17 and having a 5′-terminal guanosine residue complementary to a cytosine residue within SEQ ID NO: 17 at a position selected from the group consisting of position 292, position 297, position 302, position 338, position 341, position 347, position 359, position 361, position 364, position 366 position 372, position 394 and position 398; (iv) a probe comprising a sequence that is complementary to a portion of SEQ ID NO: 17 and having a 3′-terminal cytosine residue complementary to a guanosine residue within SEQ ID NO: 17 at a position selected from the group consisting of position 293, position 298, position 303, position 339, position 342, position 348, position 360, position 362, position 365, position 367, position 373, position 395 and position 399; and (v) a probe comprising a sequence that is complementary to any one of (i) to (iv).

As will be understood from the preceding description, exemplary combinations of diagnostic and contiguous probes that are ligatable are selected by combination of a probe set forth in sub-paragraph (i) supra with a probe set forth in sub-paragraph (ii) supra, or alternatively, by combination of a probe set forth in sub-paragraph (iii) supra with a probe set forth in sub-paragraph (iv).

Alternatively, multiple sets of diagnostic probes and multiple sets of contiguous probes can be employed to provide for assay products that detect clusters of methylated residues in the MCC gene promoter. Again, the selection of these probes can be based upon the criteria for probe design described in the preceding paragraphs.

Amplification-based Assay Format

In an alternative embodiment, the hybridization is detected using an amplification system. In methylation-specific PCR formats (MSP; Herman et al. Proc. Natl. Acad. Sci. USA 93, 9821-9826, 1992), the hybridization is detection using a process comprising amplifying the bisulphite-treated DNA. In positive read-out formats, methylation of cytosine residues within the CpG islands of these sequences in the MCC gene promoter of a colorectal cancer sample is enhanced and, as a consequence, protected from mutation. This enables enhanced annealing of one or more primers as described to the MCC gene promoter in the cancer sample thereby producing an enhanced signal relative to a normal/healthy control.

Any amplification assay format described herein can be used, such as, for example, polymerase chain reaction (PCR), rolling circle amplification (RCA), inverse polymerase chain reaction (iPCR), in situ PCR (Singer-Sam et al., Nucl. Acids Res. 18, 687,1990), strand displacement amplification, or cycling probe technology.

PCR techniques have been developed for detection of gene mutations (Kuppuswamy et al., Proc. Natl. Acad. Sci. USA 88,1143-1147, 1991) and quantitation of allelic-specific expression (Szabo and Mann, Genes Dev. 9, 3097-3108, 1995; and Singer-Sam et al., PCR Methods Appl. 1, 160-163, 1992). Such techniques use internal primers, which anneal to a PCR-generated template and terminate immediately 5′ of the single nucleotide to be assayed. Such as format is readily combined with ligase chain reaction as described herein above.

The use of a real-time quantitative assay format is particularly preferred.

Subject to the selection of appropriate primers, such assay formats are generally described herein above and apply mutatis mutandis to the presently described selective mutagenesis approach.

In a preferred embodiment, the primers are selected from the group consisting of (i) a primer comprising a sequence contained within residues from position 284 to position 304 of SEQ ID NO: 17; (ii) a primer comprising a sequence contained within residues from position 335 to position 355 of SEQ ID NO: 17; (iii) a primer comprising a sequence contained within residues from position 361 to position 382 of SEQ ID NO: 17; (v) a primer comprising a sequence contained within residues from position 383 to about position 403 or about position 404 of SEQ ID NO: 17; and (v) a primer comprising a sequence that is complementary to any one of (i) to (iv).

Amplification primers will preferably comprise sequences corresponding or complementary to any one or more of SEQ ID Nos: 18-23. For the purposes of nomenclature, the nucleotide sequence set forth in SEQ ID NO: 18 corresponds to residues at positions 284 to 304 of SEQ ID NO: 17 (i.e., in the 5-untranslated region of the MCC gene promoter from a colorectal cancer sample following bisulphite treatment of DNA). The sequence set forth in SEQ ID NO: 19 corresponds to residues at positions 335 to 355 of SEQ ID NO: 17. The sequence set forth in SEQ ID NO: 20 corresponds to the complement of residues at positions 361 to 382 of SEQ ID NO: 17 in the 5-untranslated region of the MCC gene promoter. The sequence set forth in SEQ ID NO: 21 corresponds to residues at positions 335 to 354 of SEQ ID NO: 17. The sequence set forth in SEQ ID NO: 22 corresponds to residues at positions 359 to 377 of SEQ ID NO: 17. The sequence set forth in SEQ ID NO: 23 corresponds to the complement of residues at positions 383 to about position 403 or about position 404 of SEQ ID NO: 17.

Still more preferably, for any detection format described herein that comprises an amplification step, the primers used will be a combination selected from the group consisting of:
(i) a primer comprising the sequence set forth in SEQ ID NO: 18 and a primer comprising a sequence that is the complement of SEQ ID NO: 19;
(ii) a primer comprising the sequence set forth in SEQ ID NO: 18 and a primer comprising the sequence set forth in SEQ ID NO: 20;
(iii) a primer comprising the sequence set forth in SEQ ID NO: 18 and a primer comprising a sequence that is the complement of SEQ ID NO: 21;
(iv) a primer comprising the sequence set forth in SEQ ID NO: 18 and a primer comprising a sequence that is the complement of SEQ ID NO: 22;
(v) a primer comprising the sequence set forth in SEQ ID NO: 18 and a primer comprising the sequence set forth in SEQ ID NO: 23;
(vi) a primer comprising the sequence set forth in SEQ ID NO: 19 and a primer comprising the sequence set forth in SEQ ID NO: 20;
(vii) a primer comprising the sequence set forth in SEQ ID NO: 19 and a primer comprising a sequence that is the complement of SEQ ID NO: 22;
(viii) a primer comprising the sequence set forth in SEQ ID NO: 19 and a primer comprising the sequence set forth in SEQ ID NO: 23;
(ix) a primer comprising the sequence set forth in SEQ ID NO: 20 and a primer comprising the sequence set forth in SEQ ID NO: 21;
(x) a primer comprising the sequence set forth in SEQ ID NO: 21 and a primer comprising a sequence that is the complement of SEQ ID NO: 22;
(xi) a primer comprising the sequence set forth in SEQ ID NO: 21 and a primer comprising the sequence set forth in SEQ ID NO: 23; and
(xii) a primer comprising the sequence set forth in SEQ ID NO: 22 and a primer comprising the sequence set forth in SEQ ID NO: 23.

Other combinations of suitable primers disclosed herein are also encompassed, and these will be readily identified by the skilled artisan.

It is to be understood that the detection step of the assay formats described herein clearly encompass the use of multiple rounds of amplifications and/or combinations of amplification and classical nucleic acid hybridization steps, in any order. For example, it is possible to amplify nucleic acid of the mutated (bisulphite-modified) MCC gene promoter using a combination of a primer comprising the sequence set forth in SEQ ID NO: 18 and a primer comprising the sequence of SEQ ID NO: 20, and then to hybridize the amplified nucleic acid using a labeled hybridization probe that comprises the nucleotide sequence of SEQ ID NO: 19 or a complementary sequence thereto. Similarly, it is possible to amplify nucleic acid of the mutated MCC gene promoter using a combination of a primer comprising the sequence set forth in SEQ ID NO: 21 and a primer comprising the sequence of SEQ ID NO: 23, and then to hybridize the amplified nucleic acid using a labeled hybridization probe that comprises the nucleotide sequence of SEQ ID NO: 22 or a complementary sequence thereto. The use of other hybridization probes capable of hybridizing to the amplified nucleic acid is clearly contemplated by the present invention, the only requirement being that the probe comprises a nucleotide sequence that occurs within the MCC gene promoter at a position between the two amplification primer sequences and preferably does not comprise a sequence of either amplification primer used. The skilled artisan will readily be capable of determining the nucleotide sequence of a suitable hybridization probe to perform this embodiment based upon the disclosure in SEQ ID NO: 17 and, as a consequence, the present invention is not to be limited by the precise sequence of a hybridization probe used in conjunction with amplification primers as described.

For multiple rounds of amplification, it is particularly preferred to use a nested amplification format to achieve enhanced specificity of the amplification products. For example, a first series of amplification reactions may employ a primer comprising the sequence set forth in SEQ ID NO: 18 and a primer comprising the sequence of SEQ ID NO: 20, and a second series of amplification reactions may employ: (i) a primer comprising the sequence set forth in SEQ ID NO: 18 and a primer comprising a sequence that is complementary to the sequence of SEQ ID NO: 19; or alternatively, (ii) a primer comprising the sequence set forth in SEQ ID NO: 19 and a primer comprising the sequence of SEQ ID NO: 20; or alternatively, (iii) a primer comprising the sequence set forth in SEQ ID NO: 19 and a primer comprising the sequence of SEQ ID NO: 21; or alternatively, (iv) a primer comprising the sequence of SEQ ID NO: 19 and a primer comprising the a sequence that is complementary to SEQ ID NO: 22; or alternatively, (v) a primer comprising the sequence of SEQ ID NO: 21 and a primer comprising the a sequence that is complementary to SEQ ID NO: 22. Similarly, for a first series of amplification reactions employing a primer comprising the sequence set forth in SEQ ID NO: 18 and a primer comprising the sequence of SEQ ID NO: 23, a second series of amplification reactions may employ any one or more of the sequences set forth in SEQ ID Nos: 19-22 in an appropriate combination as described supra to amplify the intervening region there between. The performance of each and every of the above-mentioned second series of amplification reactions simultaneously or contemporaneously is also encompassed by the present invention.

Other primer combinations are also not to be excluded when using multiple amplifications to detect nucleic acid, the only requirement being that the primers are selected such that they comprise nucleotide sequences that occur within the MCC gene promoter at a position between the two amplification primer sequences used for the first series of amplifications. The skilled artisan will readily be capable of determining the nucleotide sequence of suitable amplification primers to perform this embodiment based upon the disclosure in SEQ ID NO: 17 and, as a consequence, the present invention is not to be limited by the precise sequence of second round amplification primers used in conjunction with the first round amplification primers as described. Combinations of the exemplified primers set forth in SEQ ID Nos: 18-23 are preferred for first and second round amplifications because these are derived from methylated sequences of the MCC gene promoter and, as a consequence, provide enhanced specificity in the detection of hyper methylated sequences.

The use of such methylated sequence-specific primers in all detection stages also provides for validation of a primary diagnosis of colorectal cancer based on the use of a single primer set or hybridization probe, and a reduced level of false positive diagnoses than would otherwise be the case.

The present invention also encompasses the use of real-time quantitative forms of PCR, such as, for example, TaqMan (Holland et al., Proc. Natl Acad. Sci. USA, 88, 7276-7280, 1991; Lee et al., Nucleic Acid Res. 21, 3761-3766, 1993) to perform this embodiment. In adapting the TaqMan assay to this positive read-out format, it is preferred to utilize a TaqMan probe comprising the sequence set forth in SEQ ID NOs: 19 or 22 or complementary thereto labelled at its 5′- and 3′- ends with different fluorescent ligands, e.g., TAMRA and/or FAM and/or a BHQ. In a particularly preferred embodiment, the probe is labelled at the 5′-end using FAM and at the 3′-end using TAMRA. Such a probe is generally used in conjunction with amplification primers having different and non-overlapping sequences with SEQ ID NOs: 19 or 22 in the MCC gene promoter, as described herein above.

In one preferred embodiment of the TaqMan assay, the amplification primers set forth in SEQ ID Nos: 18 and 20 are used in conjunction with a TaqMan probe comprising the nucleotide sequence of SEQ ID NO: 19. In an alternative embodiment, the amplification primers set forth in SEQ ID Nos: 21 and 23 are used in conjunction with a TaqMan probe comprising the nucleotide sequence of SEQ ID NO: 22.

As with other amplification-based assay formats, the amplification product is analysed using a range of procedures, including gel electrophoresis, gel filtration, mass spectrometry, and in the case of labeled primers, by identifying the label in the amplification product. In an alternative embodiment, restriction enzyme digestion of PCR products amplified from bisulfite-converted DNA is performed essentially as described by Sadri and Hornsby, Nucl. Acids Res. 24, 5058-5059, 1996; and Xiong and Laird, Nucl. Acids Res. 25, 2532-2534, 1997), to analyze the product formed.

High throughput detection methods, such as, for example, matrix-assisted laser desorption/ionization time of flight (MALDI-TOF), electrospray ionization (ESI), Mass spectrometry (including tandem mass spectrometry, eg LC MS/MS), biosensor technology, evanescent fiber-optics technology or DNA chip technology, can also be employed.

As with the other assay formats described herein that utilize hybridization and/or amplification detection systems, combinations of such processes as described herein above are particularly contemplated by the selective mutagenesis-based assay formats of the present invention. In a particularly preferred embodiment, the enhanced methylation is detected by performing a process comprising:
(i) treating the nucleic acid with an amount of a compound that selectively mutates a non-methylated cytosine residue within a CpG island under conditions sufficient to induce mutagenesis thereby producing a mutated nucleic acid;
(ii) hybridizing the nucleic acid to two non-overlapping and non-complementary primers each of which comprises a nucleotide sequence that is complementary to a sequence in the DNA comprising a methylated cytosine residue under conditions such that hybridization to the non-mutated nucleic acid occurs;
(iii) amplifying nucleic acid intervening the hybridized primers thereby producing a DNA fragment consisting of a sequence that comprises a primer sequence;
(iv) hybridizing the amplified DNA fragment to a probe comprising a nucleotide sequence that corresponds or is complementary to a sequence comprising a methylated cytosine residue under conditions such that hybridization to the non-mutated nucleic acid occurs; and
(v) detecting the hybridization.

Based upon the teaching provided of methylated residues within the 5-untranslated region of the MCC gene promoter, the skilled artisan will readily be able to identify other probes and primers, and other combinations of primers suitable for performing the various embodiments described herein. Each of the exemplified probes and primers comprise about 1-5 CpG islands that are specifically methylated in colorectal cancer cell lines and/or Stage (C) colorectal cancer patient samples. Shorter probes or primers, sequence variants that comprise the CpG island regions of these probes, and other fimctionally-equivalent probes are also contemplated for use in the inventive diagnostic assays described herein. Other probe/primer combinations, and other primer combinations are also encompassed by the present invention.

(c)(ii) Negative Read-out Assays

In an alternative embodiment, the assay format comprises a negative read-out system in which reduced methylation of DNA from a healthy/normal control sample is detected as a positive signal and preferably, methylated DNA from a colorectal cancer sample is not detected or is only weakly detected.

In a preferred embodiment, the reduced methylation is determined using a process comprising:
(i) treating the nucleic acid with an amount of a compound that selectively mutates a non-methylated cytosine residue within a CpG island under conditions sufficient to induce mutagenesis thereby producing a mutated nucleic acid;
(ii) hybridizing the nucleic acid to a probe or primer comprising a nucleotide sequence that is complementary to a sequence comprising the mutated cytosine residue under conditions such that selective hybridization to the mutated nucleic acid occurs; and
(iii) detecting the selective hybridization.

In this context, the term “selective hybridisation” means that hybridisation of a probe or primer to the mutated nucleic acid occurs at a higher frequency or rate, or has a higher maximum reaction velocity, than hybridisation of the same probe or primer to the corresponding non-mutated sequence. Preferably, the probe or primer does not hybridise to the methylated sequence under the reaction conditions used.

For negative read-out assay formats that detect DNA from a healthy/normal control subject sample as a positive signal following treatment with bisulphite, it is preferred to use probes and/or primers derived from SEQ ID NO: 24, in which cytosine residues from the wild-type MCC gene promoter have been mutated to thymidine other than those cytosine residues within a CpG island that appears to be methylated in a healthy/normal control subject.

Hybridization-based Assay format

In one embodiment the hybridisation is detected using Southern, dot blot, slot blot or other nucleic acid hybridisation means (Kawai et al., Mol. Cell. Biol. 14, 7421-7427, 1994; Gonzalgo et al., Cancer Res. 57, 594-599, 1997). Subject to appropriate probe selection, such assay formats are generally described herein above and apply mutatis mutandis to the presently described selective mutagenesis approach.

Preferably, a ligase chain reaction format is employed to distinguish between a non-mutated and mutated MCC gene promoter. In this respect, the assay requirements and conditions are as described herein above for positive read-out assays and apply mutatis mutandis to the present format.

However the selection of probes will differ. For negative read-out assays, combinations of probes are based upon the sequence-set forth in SEQ ID NO: 24.

In an alternative embodiment of the ligase chain reaction applicable to a negative read-out, the ligated probes are detected by adding an “anchor” primer based on SEQ ID NO: 24 following the ligation reaction and performing PCR in accordance with standard amplification procedures as described herein.

Preferably, the ligase chain reaction probe(s) have 3′-terminal and/or 5′-terminal sequences that comprise a CpG island that is not methylated in a healthy control subject, but is hyper methylated in colorectal cancer, such that the diagnostic probe and contiguous probe are capable of being ligated only when the cytosine of the CpG island is mutated to thymidine e.g., in the case of a non-methylated cytosine residue.

In a preferred embodiment, the diagnostic and contiguous probes are selected from the group consisting of (i) a probe comprising a sequence contained within the sequence. set forth in SEQ ID NO: 24 and having a 5′-terminal guanosine residue at a position within SEQ ID NO: 24 selected from the group consisting of position 293, position 298, position 303, position 339, position 342, position 348, position 360, position 362, position 365, position 367, position 373, position 395 and position 399; (ii) a probe comprising a sequence contained within the sequence set forth in SEQ ID NO: 24 and having a 3′-terminal thymidine residue at a position within SEQ ID NO: 24 selected from the group consisting of position 292, position 297, position 302, position 338, position 341, position 347, position 359, position 361, position 364, position 366 position 372, position 394 and position 398; (iii) a probe comprising a sequence that is complementary to a portion of SEQ ID NO: 24 and having a 5′-terminal adenosine residue complementary to a thymidine residue within SEQ ID NO: 24 at a position selected from the group consisting of position 292, position 297, position 302, position 338, position 341, position 347, position 359, position 361, position 364, position 366 position 372, position 394 and position 398; (iv) a probe comprising a sequence that is complementary to a portion of SEQ ID NO: 24 and having a 3′-terminal cytosine residue complementary to a guanosine residue within SEQ ID NO: 24 at a position selected from the group consisting of position 293, position 298, position 303, position 339, position 342, position 348, position 360, position 362, position 365, position 367, position 373, position 395 and position 399; and (v) a probe comprising a sequence that is complementary to any one of (i) to (iv).

As will be understood from the preceding description, exemplary combinations of diagnostic and contiguous probes that are ligatable are selected by combination of a probe set forth in sub-paragraph (i) supra with a probe set forth in sub-paragraph (ii) supra, or alternatively, by combination of a probe set forth in sub-paragraph (iii) supra with a probe set forth in sub-paragraph (iv).

Alternatively, multiple sets of diagnostic probes and multiple sets of contiguous probes can be employed to provide for the detection of mutated sequences in the normal/healthy control that would otherwise be protected from mutation by methylation in colorectal cancer. Again, the selection of these probes can be based upon the criteria for probe design described in the preceding paragraphs.

Amplification-based Assay Format

In an alternative embodiment, the hybridization is detected using an amplification system using any amplification assay format as described herein above for positive read-out assay albeit using primers (and probes where applicable) that are derived from the nucleotide sequence set forth in SEQ ID NO: 24.

In negative read-out formats, mutation of non-methylated cytosine residues within the CpG islands of the MCC gene promoter of a healthy/normal subject is enhanced relative to the colorectal cancer sample. This enables enhanced annealing of one or more primers derived from SEQ ID NO: 24 to the MCC gene promoter in the bisulphite-treated control sample thereby producing an enhanced signal relative to a colorectal cancer sample.

In a preferred embodiment, the primers are selected from the group consisting of (i) a primer comprising a sequence contained within residues from position 284 to position 304 of SEQ ID NO: 24; (ii) a primer comprising a sequence contained within residues from position 335 to position 355 of SEQ ID NO: 24; (iii) a primer comprising a sequence contained within residues from position 361 to position 382 of SEQ ID NO: 24; (v) a primer comprising a sequence contained within residues from position 383 to about position 404 of SEQ ID NO: 24; and (v) a primer comprising a sequence that is complementary to any one of (i) to (iv).

Amplification primers will preferably comprise sequences corresponding or complementary to any one or more of SEQ ID Nos: 25-30. For the purposes of nomenclature, the nucleotide sequence set forth in SEQ ID NO: 25 corresponds to residues at positions 284 to about 304 of SEQ ID NO: 24 in the 5-untranslated region of a MCC gene promoter from a healthy/normal control following mutagenesis using bisulphite. The sequence set forth in SEQ ID NO: 26 corresponds to residues at positions 335 to 355 of SEQ ID NO: 24 in the 5-untranslated region of a MCC gene promoter from a healthy/normal control following mutagenesis using bisulphite. The sequence set forth in SEQ ID NO: 27 corresponds to the complement of residues at positions 361 to 382 of SEQ ID NO: 18 in the 5-untranslated region of a MCC gene promoter from a healthy/normal control following mutagenesis using bisulphite. The sequence set forth in SEQ ID NO: 28 corresponds to residues at positions 335 to 354 of SEQ ID NO: 24 in the 5-untranslated region of a MCC gene promoter from a healthy/normal control following mutagenesis using bisulphite. The sequence set forth in SEQ ID NO: 29 corresponds to residues at positions 359 to 377 of SEQ ID NO: 24 in the 5-untranslated region of a MCC gene promoter from a healthy/normal control following mutagenesis using bisulphite. The sequence set forth in SEQ ID NO: 30 corresponds to the complement of residues at positions 383 to 404 of SEQ ID NO: 18 in the 5-untranslated region of a MCC gene promoter from a healthy/normal control following mutagenesis using bisulphite. Accordingly, these primers are derived from the sequence of a non-hyper methylated MCC gene promoter that has been mutated using bisulphite.

Still more preferably, for any detection format described herein that comprises an amplification step, the primers used will be a combination selected from the group consisting of:
(i) a primer comprising the sequence set forth in SEQ ID NO: 25 and a primer comprising a sequence that is the complement of SEQ ID NO: 26;
(ii) a primer comprising the sequence set forth in SEQ ID NO: 25 and a primer comprising the sequence set forth in SEQ ID NO: 27;
(iii) a primer comprising the sequence set forth in SEQ ID NO: 25 and a primer comprising a sequence that is the complement of SEQ ID NO: 28;
(iv) a primer comprising the sequence set forth in SEQ ID NO: 25 and a primer. comprising a sequence that is the complement of SEQ ID NO: 29;
(v) a primer comprising the sequence set forth in SEQ ID NO: 25 and a primer comprising the sequence set forth in SEQ ID NO: 30;
(vi) a primer comprising the sequence set forth in SEQ ID NO: 26 and a primer comprising the sequence set forth in SEQ ID NO: 27;
(vii) a primer comprising the sequence set forth in SEQ ID NO: 26 and a primer comprising a sequence that is the complement of SEQ ID NO: 29;
(viii) a primer comprising the sequence set forth in SEQ ID NO: 26 and a primer comprising the sequence set forth in SEQ ID NO: 30;
(ix) a primer comprising the sequence set forth in SEQ ID NO: 27 and a primer comprising the sequence set forth in SEQ ID NO: 28;
(x) a primer comprising the sequence set forth in SEQ ID NO: 28 and a primer comprising a sequence that is the complement of SEQ ID NO: 29;
(xi) a primer comprising the sequence set forth in SEQ ID NO: 28 and a primer comprising the sequence set forth in SEQ ID NO: 30; and
(xii) a primer comprising the sequence set forth in SEQ ID NO: 29 and a primer comprising the sequence set forth in SEQ ID NO: 30.

It is to be understood that the detection step of the assay formats described herein clearly encompass the use of multiple rounds of amplifications and/or combinations of amplification and classical nucleic acid hybridization steps, in any order. For example, it is possible to amplify nucleic acid of the MCC gene promoter using a combination of a primer comprising the sequence set forth in SEQ ID NO: 25 and a primer comprising the sequence of SEQ ID NO: 27, and then to hybridize the amplified nucleic acid using a labeled hybridization probe that comprises the nucleotide sequence of SEQ ID NO: 26 or a complementary sequence thereto. Similarly, it is possible to amplify nucleic acid of the MCC gene promoter using a combination of a primer comprising the sequence set forth in SEQ ID NO: 28 and a primer comprising the sequence of SEQ ID NO: 30, and then to hybridize the amplified nucleic acid using a labeled hybridization probe that comprises the nucleotide sequence of SEQ ID NO: 29 or a complementary sequence thereto. The use of other hybridization probes capable of hybridizing to the amplified nucleic acid is clearly contemplated by the present invention, the only requirement being that the probe comprises a nucleotide sequence that occurs within the MCC gene promoter at a position between the two amplification primer sequences and preferably does not comprise a sequence of either amplification primer used. The skilled artisan will readily be capable of determining the nucleotide sequence of a suitable hybridization probe to perform this embodiment based upon the disclosure in SEQ ID NO: 3 and, as a consequence, the present invention is not to be limited by the precise sequence of a hybridization probe used in conjunction with amplification primers as described.

For multiple rounds of amplification, it is particularly preferred to use a nested amplification format to achieve enhanced specificity of the amplification products. For example, a first series of amplification reactions may employ a primer comprising the sequence set forth in SEQ ID NO: 25 and a primer comprising the sequence of SEQ ID NO: 27, and a second series of amplification reactions may employ: (i) a primer comprising the sequence set forth in SEQ ID NO: 25 and a primer comprising a sequence that is complementary to the sequence of SEQ ID NO: 26; or alternatively, (ii) a primer comprising the sequence set forth in SEQ ID NO: 26 and a primer comprising the sequence of SEQ ID NO: 27; or alternatively, (iii) a primer comprising the sequence set forth in SEQ ID NO: 26 and a primer comprising the sequence of SEQ ID NO: 28; or alternatively, (iv) a primer comprising the sequence of SEQ ID NO: 26 and a primer comprising the a sequence that is complementary to SEQ ID NO: 29; or alternatively, (v) a primer comprising the sequence of SEQ ID NO: 28 and a primer comprising the a sequence that is complementary to SEQ ID NO: 29. Similarly, for a first series of amplification reactions employing a primer comprising the sequence set forth in SEQ ID NO: 25 and a primer comprising the sequence of SEQ ID NO: 30, a second series of amplification reactions may employ any one or more of the sequences set forth in SEQ ID Nos: 26-29 in an appropriate combination as described supra to amplify the intervening region there between. The performance of each and every of the above-mentioned second series of amplification reactions simultaneously or contemporaneously is also encompassed by the present invention.

For combinations of amplification and classical nucleic acid hybridization steps, it is preferred to use a combination of a primer comprising the sequence set forth in SEQ ID NO: 25 and a primer comprising the sequence of SEQ ID NO: 27, and then to hybridize the amplified nucleic acid using a labeled hybridization probe that comprises the nucleotide sequence of SEQ ID NO: 26 or a complementary sequence thereto. Alternatively, a combination of a primer comprising the sequence set forth in SEQ ID NO: 28 and a primer comprising the sequence of SEQ ID NO: 30 is used for amplification, and the amplified nucleic acid is hybridized to a labeled hybridization probe that comprises the nucleotide sequence of SEQ ID NO: 29 or a complementary sequence thereto. The use of other hybridization probes capable of hybridizing to the amplified nucleic acid is clearly contemplated by the present invention, the only requirement being that the probe comprises a nucleotide sequence that occurs within the MCC gene promoter at a position between the two amplification primer sequences and preferably does not comprise a sequence of either amplification primer used. The skilled artisan will readily be capable of determining the nucleotide sequence of a suitable hybridization probe to perform this embodiment based upon the disclosure in SEQ ID NO: 24 and, as a consequence, the present invention is not to be limited by the precise sequence of a hybridization probe used in conjunction with amplification primers as described.

In adapting the TaqMan assay to this positive read-out format, it is preferred to utilize a TaqMan probe comprising the sequences set forth in SEQ ID NOs: 26 or 29 labelled at its 5′- and 3′-ends with different fluorescent ligands, e.g., TAMRA and/or FAM and/or a BHQ. In a particularly preferred embodiment, the probe is labelled at the 5′-end using FAM and at the 3′-end using TAMRA. Such a probe is generally used in conjunction with amplification primers having different and non-overlapping sequences with SEQ ID NOs: 26 or 29 in the MCC gene promoter, as described herein above.

In one preferred embodiment of the TaqMan assay, the amplification primers set forth in SEQ ID Nos: 25 and 27 are used in conjunction with a TaqMan probe comprising the nucleotide sequence of SEQ ID NO: 26. In an alternative embodiment, the amplification primers set forth in SEQ ID Nos: 28 and 30 are used in conjunction with a TaqMan probe comprising the nucleotide sequence of SEQ ID NO: 29.

In a particularly preferred embodiment, the reduced methylation in the normal/healthy control subject is detected by performing a process comprising:
(i) treating the nucleic acid with an amount of a compound that selectively mutates non-methylated cytosine residues under conditions sufficient to induce mutagenesis thereby producing a mutated nucleic acid;
(ii) hybridizing the nucleic acid to two non-overlapping and non-complementary primers each of which comprises a nucleotide sequence that is complementary to a sequence in the DNA comprising a mutated cytosine residue under conditions such that hybridization to the mutated nucleic acid occurs;
(iii) amplifying nucleic acid intervening the hybridized primers thereby producing a DNA fragment consisting of a sequence that comprises a primer sequence;
(iv) hybridizing the amplified DNA fragment to a probe comprising a nucleotide sequence that corresponds or is complementary to a sequence comprising a mutated cytosine residue under conditions such that hybridization to the mutated nucleic acid occurs; and
(v) detecting the hybridization.
   d) Multiplex Assay Formats

Improved accuracy of diagnosis is also obtained by multiplexing the assay of the invention with other diagnostic platforms as described herein (eg., hypermethylation of a gene selected from the group consisting of HLTF, APC, p16^{INK4a},p14^{ARF}, HPP1, hMLH1, MGMT, and combinations thereof) and/or methylation of 3-4 CIMP markers and/or a deletion or point mutation of APC or DCC and/or mutation of HLTF and/or one or more tumor suppressor genes selected from the group consisting of pRB, p53, WT1, NF1, NF2, a ERM family protein). Naturally, when used in combination with the assay of the present invention, population coverage may be compromised for such improved accuracy.

Accordingly, the present invention provides multiplexed assays comprising the detection of hypermethylation in the MCC gene promoter in combination with the detection of hypermethylation within a gene selected from the group consisting of HLTF, APC, p16^{INK4a},p14^{ARF}, HPP1, hMLH1, MGMT, and combinations thereof. Alternatively, or in addition, this embodiment provides for the detection of hyper methylation in the MCC gene promoter in combination with a determination of a a CpG island methylation phenotype (CIMP) of the sample. Alternatively, or in addition, this embodiment provides for the detection of hyper methylation in the MCC gene promoter in combination with a determination of a mutation within the DCC gene and/or APC gene.

Detection of methylated CpG sites within the 5′ region of the p16^{INKa} gene is preferably performed using MS PCR essentially as described by Clark et al., Nucleic Acids Res. 22, 2990-2997, 1994 and/or Herman et al., Proc. Natl. Acad. Sci. USA, 93, 9821-9826, 1996. Briefly, about 1.0 μg of purified DNA is diluted in 0.3N NaOH and DNA denatured at 37° C. for about 15 min. The sample is then treated with sodium bisulfite solution pH 5.0) and hydroquinone prepared fresh for each analysis. Samples are incubated at about 55° C. for about 16 h, in the presence of an overlay of mineral oil to prevent evaporation. The solution is then cooled to −80° C. for about 10 min, the mineral oil removed, and bisulfite-modified DNA purified e.g., using the Wizard DNA Clean-UP System and Vacuum Manifold (Promega) according to the manufacturer's instructions. Cytosine-to-uracil conversion is achieved using alkali (e.g., 0.3N NaOH) at 37° C. for about 15 min. A methylation-positive colorectal cancer cell line (e.g., SW-480; American Type Culture Collection) and/or a methylation-negative cell line (e.g., SK-N-SH; American Type Culture Collection) can be used as controls for the bisulfite conversion, DNA recovery, and PCR reactions (as with assays for MCC promoter methylation or methylation of any other gene described herein). For each polyp or tumor sample, bisulfite-treated DNA is amplified using commercially available primers (Life Technologies, Inc.) specific for methylated (M primers) and nonmethylated (U primers) CpG sites within the p16^{INK4a} promoter region; (M primers: 5-TTATTAGAGGGTGGGGCGGATCGC (SEQ ID NO: 35) and 5-GACCCCGAACCGCGACCGTAA (SEQ ID NO: 36); and U primers: 5-TTATTAGAGGGTGGGGTGGATTGT (SEQ ID NO: 37) and 5-CCACCTAAATCAACCTCCAACCA (SEQ ID NO: 38). Standard PCR reaction conditions are used (e.g., GeneAmp PCR buffer, Perldn-Elmer Corp.; 1.5 mM MgCl₂; 200 μM each deoxynucleotide triphosphate; 0.4 μM each primer; 50 ng modified DNA template; and 2.5 units of AmpliTaq, Perkin-Elmer Corp. in a total volume of 50 μl; preheated at 94° C. for 1 min, then 30-35 cycles of 94° C. for 30 s, 65° C. for 10-30 s, 72° C. for 30 s, and a final extension at 72° C. for 10 min).

Methods used to determine methylation of p16^{ARF14} are also suitable for determining the methylation of other genes multiplexed with MCC.

Detection of methylated CpG sites within the 5′ region of the HPPI gene is preferably performed using MS PCR essentially as described above using the primers:

5-GTTATCGTCGTCGTITTTGTTGTC-3′ (SEQ ID NO: 39) and 5-GACTTCCGAAAAACACAAAATCG-3′ (SEQ ID NO: 40). The amplification product is preferably detected by hybridizing a probe comprising the sequence 5-6FAM-CCGAACAACGAACTACTAAACATCCCGCG-TAMRA -3′ (SEQ ID NO: 41) to the amplification product.

Detection of methylated CpG sites within the 5′ region of the HMLHI gene is preferably performed using MS PCR essentially as described above using the primers:

5-TAATCTATCGCCGCCTCATCG-3′ (SEQ ID NO: 42) and 5-TCGTTATATATCGTTCGTAGTATTCGTGTTTAGTTC-3′ (SEQ ID NO: 43). The amplification product is preferably detected by hybridizing a probe comprising the sequence 5-6FAM-CGAACGCGACGTCAAACGCCACTA-TAMRA -3′ (SEQ ID NO: 44) to the amplification product.

Detection of methylated CpG sites within the 5′ region of the O⁶-Methylguanine-DNA methyltransferase (MGMT) gene is preferably performed using MS PCR essentially as described above using the primers:

5-TTTCGACGTTCGTAGGTTTCGC-3 (SEQ ID NO: 45) and 5-GCACTCTTCCGAAAACGAAACG-3′ (SEQ ID NO: 46). The amplification product is preferably detected by hybridizing a probe to the amplification product.

For determining CIMP, it is preferred to use MCA essentially as described by Toyota et al., Cancer Res. 59, 2307-2312, 1999. Briefly, a fragment of genomic DNA is digested with the methylation sensitive SmaI enzyme to remove unmethylated SmaI sites, and produce a blunt ended fragment. Methylated SmaI sites are then digested with the nonmethylation-sensitive isoschizomer XmaI, which digests methylated CpG islands within SmaI recognition sites, leaving a CCGG overhang. Adaptors are ligated to these overhangs, and PCR is performed to amplify the methylated sequences. The MCA amplicons are hybridizaed to one or more MINT probes e.g., in a dot blot analysis. Preferably, the MINT1, MINT2, MINT12 and MINT 31 probes are employed.

Preferably, signals are generated using at least 3 or 4 MINT probes.

As used herein, the term “HLTF gene” shall be taken to mean a nucleic acid, including any genomic gene, that is linked to or positioned at map position 3q24-q25 of the human genome, or any MRNA transcript thereof, or any genomic gene or mRNA transcript from a human or non-human animal that comprises a nucleotide sequence having at least about 80% identity to the sequence of a human HTFF gene transcript as set forth in SEQ ID NO: 4 or encoding a polypeptide having an amino acid sequence that is at least about 60% identity to a sequence as set forth in SEQ ID NO: 5 or 6 and preferably exhibits an activity characteristic of HLTF by virtue of HLTF belonging to the SWI/SNF family of transcriptional activators.

For the purposes of nomenclature, the nucleotide sequence of the open reading frame, 5-untranslated region and 3-untranslated region of the HLTF gene (Ding et al., DNA Cell Biol 15, 429-442, 1996, incorporated herein by reference) is set forth herein as SEQ ID NO: 4. It is known that there are two HLTF proteins encoded by SEQ ID NO: 4 which are the products of alternate translation initiation.

As used herein, the term “DCC gene” or “CRC18 gene” or “CRCR1 gene” shall be taken to mean a nucleic acid, including any genomic gene, that is linked to or positioned at map position 18q21.3 of the human genome, or any mRNA transcript thereof, or any genomic gene or MRNA transcript from a human or non-human animal that comprises a nucleotide sequence having at least about 80% identity to the sequence of a human DCC gene transcript as set forth in SEQ ID NO: 7 or encoding a polypeptide having an amino acid sequence that is at least about 60% identity to a sequence as set forth in SEQ ID NO: 8.

For the purposes of nomenclature, the nucleotide sequence of the open reading frame and 3-untranslated region of the human DCC gene (O'Boyle et al., Cancer Invest. 21, 484-485, 2003; Bamias et al., Cancer Invest. 21, 333-340, 2003; Hedrick et al., Genes Devel. 8, 1174-1183, 1994; Cho et al., Genomics 19, 525-531, 1994; and Fearon et al., Science 247, 49-56, 1990 each of which is incorporated herein by reference) is set forth herein as SEQ ID NO: 7. The amino acid sequence of the corresponding encoded DCC polypeptide is set forth as SEQ ID NO: 8.

As used herein, the term “APC. gene” shall be taken to mean a nucleic acid, including any genomic gene, that is linked to or positioned at map position 5q21 of the human genome, or any mRNA transcript thereof, or any genomic gene or MRNA transcript from a human or non-human animal that comprises a nucleotide sequence having at least about 80% identity to the sequence of a human APC gene transcript as set forth in SEQ ID NO: 9 or encoding a polypeptide having an amino acid sequence that is at least about 60% identity to a sequence as set forth in SEQ ID NO: 10.

For the purposes of nomenclature, the nucleotide sequence of the 5-untranslated region, open reading frame, and 3-untranslated region of the human APC gene (Su et al., Hum. Genet. 106, 101-107, 2000; Vogelstein et al., U.S. Pat. Reissue No. RE36,713 May 23, 2000; Albertsen et al., U.S. Pat. No. 5,648,212 Jul. 15, 1997; each of which is incorporated herein by reference) is set forth herein as SEQ ID NO: 9. The amino acid sequence of the corresponding encoded APC polypeptide is set forth as SEQ ID NO: 10.

The skilled artisan will readily be in a position to determine a mutation in a DCC and/or an APC gene based on the sequence disclosures herein and/or information available in the public domain.

Any multiplex/multianalyte assay format described herein that involve detection of MCC hypermethylation will enhance the rate of positive detection of early stage colorectal cancer to about 80% or higher, and strengthen the predictive value of tests for determining a malignant potential of a polyp, especially HP.

In one embodiment, probes and primers specific for genes other than MCC are combined with primers and probes for MCC in single reactions to identify diagnostic methylation patterns in both gene promoters simultaneously. The only requirement for such approaches is that the probes/primers are separately labelled or otherwise separately detectable. Alternatively, reactions are performed separately using the same samples as starting material.

In an alternative embodiment, the detection of hyper methylation within the MCC gene promoter is combined with detection of mutations within the APC gene essentially as described in U.S. Pat. No. 5,648,212 and /or by Traverso et al., New England J. Med 346, 311-320, 2002.

In an alternative embodiment, the detection of hyper methylation within the MCC gene promoter is combined with detection of mutations within the DCC gene.

The present invention clearly encompasses any and all combinations of such multiplex assay formats.

Biological Samples

The present invention is particularly applicable to non-invasive detection of colorectal cancer. Accordingly, suitable samples include bodily fluids and stool samples. DNA can be prepared readily from all such samples using standard procedures. However, stool samples are known to contain various inhibitors of DNA polymerase and, as a consequence, it is preferred to capture the DNA from such samples onto magnetic beads coated with suitable probes to permit amplification of a hyper methylated region of the MCC gene promoter e.g., between nucleotide positions 284 and 404 of SEQ ID Nos: 3, 17 or 24. The isolation of DNA from stool samples is described by Traverso et al., for amplification of the APC gene between codons 1210 and 1581.

Bodily fluids shall be taken to include whole blood, serum, peripheral blood mononuclear cells (PBMC), buffy coat fraction, urine, semen, or abdominal fluid.

In an alternative embodiment, the biological sample will comprise colorectum tissue derived preferably by colonoscopy, or cells, and those tissues known to comprise cancer cells arising from a micrometastasis of a colorectal cancer.

In the present context, the term “cancer cell” includes any biological specimen or sample comprising a cancer cell irrespective of its degree of isolation or purity, such as, for example, tissues, organs, cell lines, bodily fluids, or histology specimens that comprise a cell in the early stages of transformation or having been transformed.

As the present invention is particularly useful for the early detection of cancer in the medium to long term, the definition of “cancer cell” is not to be limited by the stage of a cancer in the subject from which said cancer cell is derived (ie. whether or not the patient is in remission or undergoing disease recurrence or whether or not the cancer is a primary tumor or the consequence of metastases). Nor is the term “cancer cell” to be limited by the stage of the cell cycle of said cancer cell.

Preferably, the biological sample has been isolated previously from the subject. In such cases, the sample may be processed or partially processed into a nucleic acid sample that is substantially free of contaminating protein. All such embodiments are encompassed by the present invention.

Control Assay Samples

It will be apparent from the preceding description that the diagnostic method provided by the present invention involves a degree of quantification to determine elevated or enhanced methylation of nucleic acid in tissue that is suspected of comprising a colorectal tumour cell or metastases thereof. Such quantification is readily provided by the inclusion of appropriate control samples in the assays as described below.

As will be known to the skilled artisan, when internal controls are not included in each assay conducted, the control may be derived from an established data set.

Data pertaining to the control subjects are selected from the group consisting of:
1. a data set comprising measurements of the degree of methylation for a typical population of subjects known to have colorectal cancer;

2. a data set comprising measurements of the degree of methylation for the subject being tested wherein said measurements have been made previously, such as, for example, when the subject was known to healthy or, in the case of a subject having colorectal cancer, when the subject was diagnosed or at an earlier stage in disease progression;
3. a data set comprising measurements of the degree of methylation for a healthy individual or a population of healthy individuals;
4. a data set comprising measurements of the degree of methylation for a normal individual or a population of normal individuals; and
5. a data set comprising measurements of the degree of methylation from the subject being tested wherein the measurements are determined in a matched sample.

Those skilled in the art are readily capable of determining the baseline for comparison in any diagnostic assay of the present invention without undue experimentation, based upon the teaching provided herein.

In the present context, the term “typical population” with respect to subjects known to have colorectal cancer shall be taken to refer to a population or sample of subjects diagnosed with colorectal cancer that is representative of the spectrum of colorectal cancer patients. This is not to be taken as requiring a strict normal distribution of morphological or clinicopathopathological parameters in the population, since some variation in such a distribution is permissible. Preferably, a “typical population” will exhibit a spectrum of colorectal cancers at different stages of disease progression and with tumours at different stages and having different morphologies or degrees of differentiation. It is particularly preferred that a “typical population” exhibits the expression characteristics of a cohort of subjects or non-cancerous cell lines as described herein.

In the present context, the term “healthy individual” shall be taken to mean an individual who is known not to suffer from colorectal cancer, such knowledge being derived from clinical data on the individual, including, but not limited to, a different colorectal cancer diagnostic assay to that described herein. As the present invention is particularly useful for the early detection of colorectal cancer or survival, it is preferred that the healthy individual is asymptomatic with respect to the any symptoms associated with colorectal cancer.

The term “normal individual” shall be taken to mean an individual having a normal level of methylation or expression of a colorectal cancer-associated gene or a normal level of expression of a colorectal cancer-associated protein in a particular sample derived from said individual. As will be known to those skilled in the art, data obtained from a sufficiently large sample of the population will normalize, allowing the generation of a data set for determining the average level of a particular parameter.

Accordingly, the level of methylation or expression of a colorectal cancer-associated gene or the level of expression of a colorectal cancer-associated protein can be determined for any population of individuals, and for any sample derived from said individual, for subsequent comparison to levels determined for a sample being assayed. Where such normalized data sets are relied upon, internal controls are preferably included in each assay conducted to control for variation.

The term “matched sample” shall be taken to mean that a control sample is derived from the same subject as the test sample is derived, at approximately the same point in time. Preferably, the control sample shows little or no morphological and/or pathological indications of colorectal cancer. Matched samples are not applicable to blood-based or serum-based assays. Accordingly, it is preferable that the matched sample is a region of a colon, colon wall or lining, abdominal cavity, derived from a subject suffering from colorectal cancer, wherein that sample does not include malignant cells or exhibit any symptom of the disease (e.g., no detectable polyposis, carcinoma in situ, tumor or metastases as determined by art-recognized methods such as, for example, biopsy of resected colon). Preferably, the sample comprises less than about 20% malignant colorectal cells, more preferably less than about 10% malignant colorectal cells, even more preferably less than about 5% malignant colorectal cells and most preferably less than about 1% malignant colorectal cells. Morphological and pathological indications of malignant colorectal cells are known in the art and/or described herein.

MCC Probes/Primers

A further aspect of the present invention provides an isolated nucleic acid probe or primer that is capable of selectively hybridising to a region of the MCC gene promoter that is hyper methylated in a colorectal cancer, wherein said region comprises or is contained within nucleotide residues from about position 284 to about position 404 of SEQ ID NO: 3 or SEQ ID NO: 17 or SEQ ID NO: 24.

In those cases where the diagnostic probe and the contiguous probe are not already available, they must be synthesized. Apparatus for such synthesis is presently available commercially, such as the Applied Biosystems 380A DNA synthesizer and techniques for synthesis of various nucleic acids are available in the literature.

In one embodiment, the probes are prepared for ligation, e.g., if ligase is to be used, the probe which will have its 5′ end adjacent the 3′ end of the other probe when hybridized to the sample nucleic acid is phosphorylated in order to later be able to form a phosphodiester bond between the two probes. One of the probes is then labeled. This labeling can be done as part of the phosphorylation process above using radioactive phosphorus, or can be accomplished as a separate operation by covalently attaching chromophores, fluorescent moieties, enzymes, antigens, chemiluminescent moieties, groups with specific binding activity, or electrochemically detectable moieties, etc. such as, for example, using T4 polynucleotide kinase.

In accordance with this embodiment, preferred hybridisation probes are selected from the group consisting of
(i) a probe having a nucleotide sequence set forth in SEQ ID NO: 3 or comprising nucleotide residues from about position 284 to about position 403 or about position 404 of SEQ ID NO: 3;
(ii) a probe having a nucleotide sequence set forth in SEQ ID NO: 17 or comprising nucleotide residues from about position 284 to about position 403 or about position 404 of SEQ ID NO: 14;
(iii) a probe having a nucleotide sequence set forth in SEQ ID NO: 24 or comprising nucleotide residues from about position 284 to about position 403 or about position 404 of SEQ ID NO: 18;
(iv) a probe of at least about 18 nucleotides in length and comprising a sequence contained within the sequence set forth in SEQ ID NO: 17 and having a 5′-terminal guanosine residue at a position within SEQ ID NO: 17 selected from the group consisting of position 293, position 298, position 303, position 339, position 342, position 348, position 360, position 362, position 365, position 367, position 373, position 395 and position 399;
(v) a probe of at least about 18 nucleotides in length and comprising a sequence contained within the sequence set forth in SEQ ID NO: 17 and having a 3′-terminal cytosine residue at a position within SEQ ID NO: 17 selected from the group consisting of position 292, position 297, position 302, position 338, position 341, position 347, position 359, position 361, position 364, position 366 position 372, position 394 and position 398;
(vi) a probe of at least about 18 nucleotides in length and comprising a sequence that is complementary to a portion of SEQ ID NO: 17 and having a 5′-terminal guanosine residue complementary to a cytosine residue within SEQ ID NO: 17 at a position selected from the group consisting of position 292, position 297, position 302, position 338, position 341, position 347, position 359, position 361, position 364, position 366 position 372, position 394 and position 398;
(vii) a probe of at least about 18 nucleotides in length and comprising a sequence that is complementary to a portion of SEQ ID NO: 17 and having a 3′-terminal cytosine residue complementary to a guanosine residue within SEQ ID NO: 17 at a position selected from the group consisting of position 293, position 298, position 303, position 339, position 342, position 348, position 360, position 362, position 365, position 367, position 373, position 395 and position 399;
(viii) a probe of at least about 18 nucleotides in length and comprising a sequence contained within the sequence set forth in SEQ ID NO: 24 and having a 5′-terminal guanosine residue at a position within SEQ ID NO: 24 selected from the group consisting of position 293, position 298, position 303, position 339, position 342, position 348, position 360, position 362, position 365, position 367, position 373, position 395′ and position 399;
(ix) a probe of at least about 18 nucleotides in length and comprising a sequence contained within the sequence set forth in SEQ ID NO: 24 and having a 3′-terminal cytosine residue at a position within SEQ ID NO: 24 selected from the group consisting of position 292, position 297, position 302, position 338, position 341, position 347, position 359, position 361, position 364, position 366 position 372, position 394 and position 398;
(x) a probe of at least about 18 nucleotides in length and comprising a sequence that is complementary to a portion of SEQ ID NO: 24 and having a 5′-terminal guanosine residue complementary to a cytosine residue within SEQ ID NO: 24 at a position selected from the group consisting of position 292, position 297, position 302, position 338, position 341, position 347, position 359, position 361, position 364, position 366 position 372, position 394 and position 398;
(xi) a probe of at least about 18 nucleotides in length and comprising a sequence that is complementary to a portion of SEQ ID NO: 24 and having a 3′-terminal cytosine residue complementary to a guanosine residue within SEQ ID NO: 24 at a position selected from the group consisting of position 293, position 298, position 303, position 339, position 342, position 348, position 360, position 362, position 365, position 367, position 373, position 395 and position 399;
(xii) a probe comprising a sequence that is complementary to any one of (i) to (xi).

Particularly preferred amplification primers comprise a nucleotide sequence selected from the group consisting of SEQ ID Nos: 11, 12, 13, 14, 15, 16, 18, 19, 20, 21, 22, 23, 25, 26, 27, 28, 29 and 30, and sequences complementary thereto.

The present invention is further described with reference to the following non-limiting examples and the accompanying drawings.

### EXAMPLE 1

### Loss of Expression of the MCC Gene in Colon Cancer Correlates with Hypermethylation of the MCC Gene Promoter

Materials and Methods

Patients

Stage C colon cancer patients operated between 1971 and 1999 were identified. The cancers were resected and the patient outcome was followed up for a minimum of 4 years. For this study, patients were selected who had not received any adjuvant chemotherapy. All rectal, sigmoid and metachronous cancers as well as ulcerative colitis patients were excluded. Archival pathology specimens were re-evaluated for pathology and tumour and the corresponding normal tissue specimens (normal lymph node or colon tissue) were manually microdissected from H&E stained serial sections. DNA was successfully purified from 103 cases using the Puregene DNA Isolation Kit (Gentra Systems).

Bisulphite Sequencing.

DNA from the colorectal cancer cell lines HCT116 (ATCC CCL-247), HCT15 (ATCC CCL-225), SW620 (ATCC CCL-227), SW116 (ATCC CCL-233), LS411N (ATCC CRL-2159), LS513 (CRL-2134), LoVo (ATCC CCL-229), CaCo-2 (ATCC HTB-37), Co-115, HPR600, LIM1215, Lisp 1, KM12SM, and the ovarian cancer cell line TOV21G was bisulphite-treated using standard methods (Herman et al., Proc Natl Acad Sci USA., 93(18), 9821-9826, 1996). The MCC promoter region containing a CpG island was amplified using primers MCC-promF 5′-GGTTAGTAGTTAGATAGTTGT-3′ (SEQ ID NO: 31), and MCC-promR 5′-TACTTAATCCCTTCTACCAC-3′ (SEQ ID NO: 32). PCR conditions were 95° C. (30s), 53° C. (45s), 72° C. (90s) for 40 cycles with an initial denaturation at 95° C. for 12 min and final elongation at 72° C. for 7 min. All PCR reactions in this study were carried out using AmpliTaq Gold (Applied Biosystems) in a DNA engine DYAD™ (MJ Research).

A 514-bp fragment was detected in all cell lines and sequenced using BigDye on an ABI PRISM 3700 DNA Analyzer (Applied Biosystems).

Methylation Specific PCR

A densely methylated section of the MCC promoter was amplified from bisulphite-treated patient specimens and cell line DNA using methylation specific primers MCC-metF1 5′-TATTGTTTCGGAACGGGGCGT-3′ (SEQ ID NO: 18) and MCC-metR1 5′-CAAAAAACTCGATAACGCGACG-3′ (SEQ ID NO: 20). PCR conditions were 95° C. (30s), 61° C. (45s), 72° C. (30s) for 35 cycles. The cycling conditions included an initial denaturation step at 95° C. for 12 min and final elongation at 72° C. for 7 min. The predicted fragment size of the amplification product was about 99 bp.

For control reactions, MYOD1 gene primers were used to amplify a MYOD1 gene fragment (Usadel et al., Cancer Res. 62(2), 371-375, 2002) in a region of the gene that does not contain any CpG nucleotide sequences. Accordingly, this control reaction ensured integrity of each DNA specimen. PCR conditions were 95° C. (45s), 57° C. (45s), 72° C. (60s) for 40 cycles.

The p16 gene promoter was amplified using primers for the methylated sequences as previously described (Herman et al., Prc. Natl Acad. Sci. USA 93, 9821-9826, 1996). PCR conditions included 40 cycles of amplification wherein each cycle consisted of 95° C. for 30 s, 65° C. for 30 s, and 72° C. for 30 s. These 40 cycles were flanked by an initial denaturation step at 95° C. for 12 min, and a final elongation step at 72° C. for 7 min. PCR reactions were carried out using AmpliTaq Gold (Applied Biosystems) in a DNA engine DYAD (MJ. Reasearch).

All methylation-specific PCR products were visualised on 6% polyacrylamide gels.

Methylation Specific Real-time PCR

The primers MCC-met-1 and MCC-metR1 were also used in a Real-time PCR together with the methylated amplicon-specific fluorogenic hybridisation probe FAM5′-TTTCGTCGTTGTCGTAGTTGC-3′ TAMRA (SEQ ID NO: 20). The PCR was carried out using the TaqMan PCR buffer A pack (Applied Biosystems) in a Rotorgene 3000 (Corbett Research).

In an alternative embodiment, TaqMan assays are performed using the amplification primers set forth in SEQ ID Nos: 21 and 23, with a TaqMan probe based upon the nucleotide sequence set forth in SEQ ID NO: 22, appropriately labeled at the 5-end using FAM and at the 3-end using BHQ-1. The PCR was carried out using the

REalMasterMix Probe ROX (Eppendorf) with 4 mM MgCl₂ in a Rotorgene 3000 (Corbett Research). PCR conditions included an initial denaturation at 95° C. for 2 min, followed by 50 cycles wherein each cycle consisted of 95° C. for 15 s and 60° C. for 60 s. All specimens were also amplified with the primers and a TaqMan probe (Usadel et al., Cancer Res. 62, 371-375, 2002) specific for the MYOD control gene sequences referred to herein above, to ensure that each specimen had been bisulphite converted regardless of methylation status. The same PCR conditions were used for MYOD as for MCC.

cDNA Analysis.

RNA was extracted from cell lines HCT-115, LS411N and TOV21G using an RNeasy Maxi kit (Qiagen). cDNA was prepared with the Expand Reverse Transcriptase kit (Roche). A 265-bp fragment was amplified from exons 1 and 2 using primers MCC-cDNAF1 5′-CTGGGTGAAAATGGCTGTCT-3′ (SEQ ID NO: 33) and MCC-cDNAR1 5′-GTTCCCTCTCTGTTTGCTGG-3′ (SEQ ID NO: 34). PCR conditions were 95° C. (30s), 58° C. (60s), 72° C. (50s) for 35 cycles with an initial denaturation step at 95° C. for 12 min and final elongation at 72° C. for 7 min.

A housekeeping gene ALAS-1 was used for an internal reference PCR (422 bp fragment) to test for the integrity of the cDNA specimens in the same conditions.

Results

CpG island Methylation of the MCC Promoter.

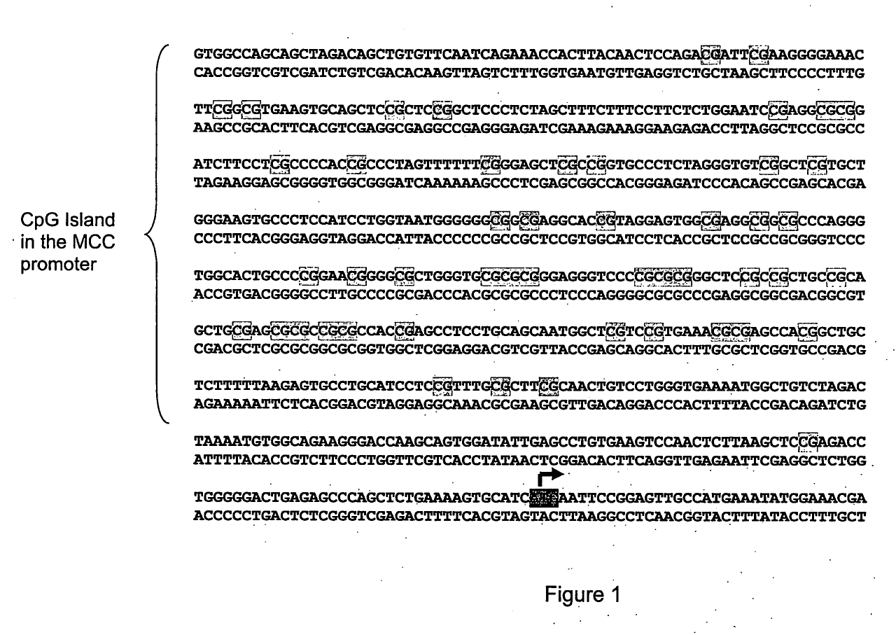

A MCC cDNA sequence obtained from GenBank (BC009279) was amended and extended 5′ using further sequence data from the Ensembl Genome Browser located at the URL ensemble.org. The sequence discrepancy involved nucleotides 1-7 of the GenBank entry. A 403-bp section of the MCC promoter (−542 to −139) was found to contain 48 CpG sites (FIG. 1).

Bisulphite sequencing of the promoter revealed extensive methylation in six colon cancer cell lines, Lisp 1, Co-115, LS513, KM12SM, LS411N, HCT15.

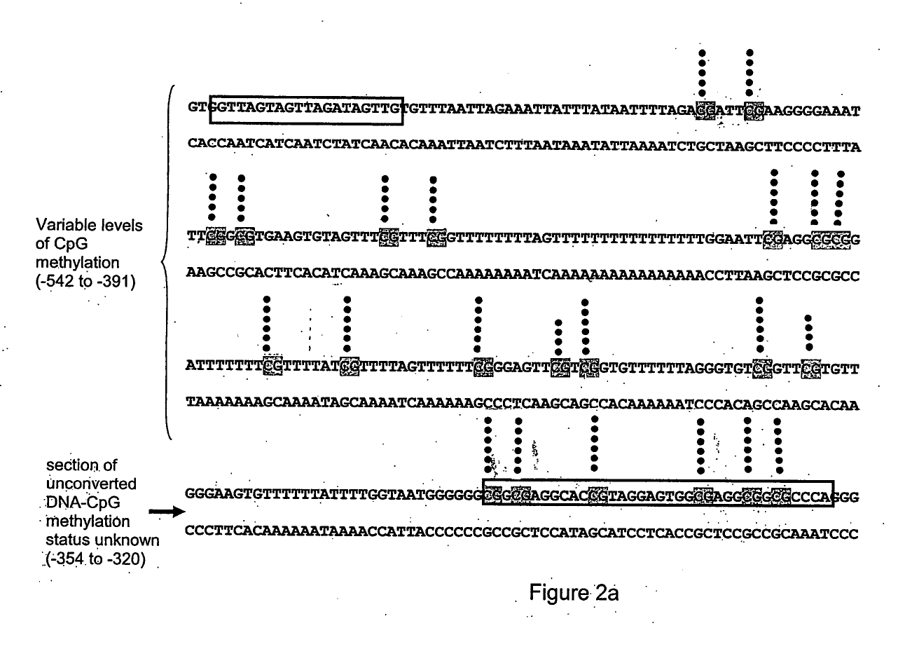

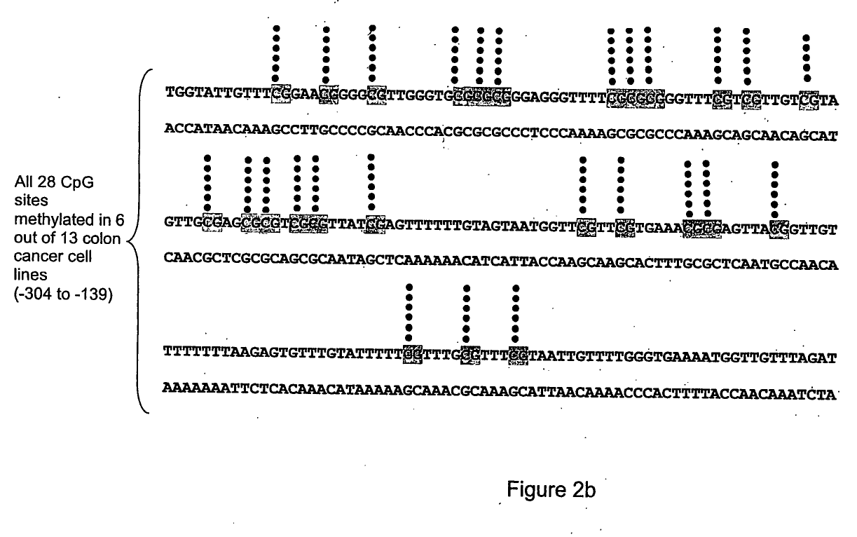

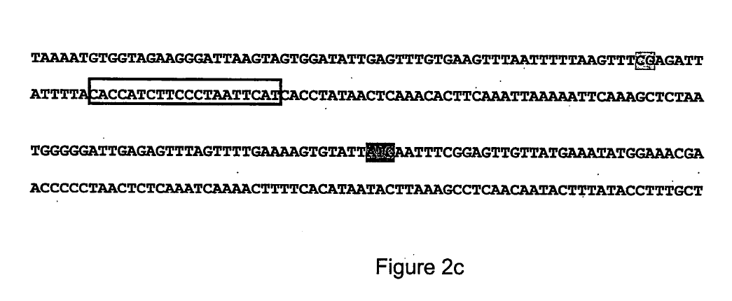

The majority of the CpG sites in the other cell lines studied (SW620, HPR600, SW116, CaCo2, LoVo, HCT116, LIM1215, TOV21G) were bisulphite-converted, except for six sites in the middle of the CpG island. These six sites showed a partial or a complete lack of bisulphite conversion, which also affected the other five C nucleotides in this region (−354 to −320; FIG. 2). One further CpG site (−269) outside this region showed partial methylation in six of the eight “unmethylated” cell lines.

The section of unconverted DNA appeared to divide the CpG island into two identifiably distinct regions:
(i) a region where methylation was consistently detected in all 26 CpG sites (region −304 to −139) for colon cancer cell lines; and
(ii) a region where methylation was variable (region −542 to −391) in those colorectal cancer cell lines that exhibited hiethylation in region (i). For example, all 10 CpG sites in region −465 to −391 showed only partial methylation in the cell line LS411N, ie. every site showed both methylated and unmethylated sequence peaks of varying intensity. In contrast, the cell line HCT15 was more completely methylated in this region with only 2 out of the 10 sites partially unmethylated.
   RNA Expression of the MCC Gene.

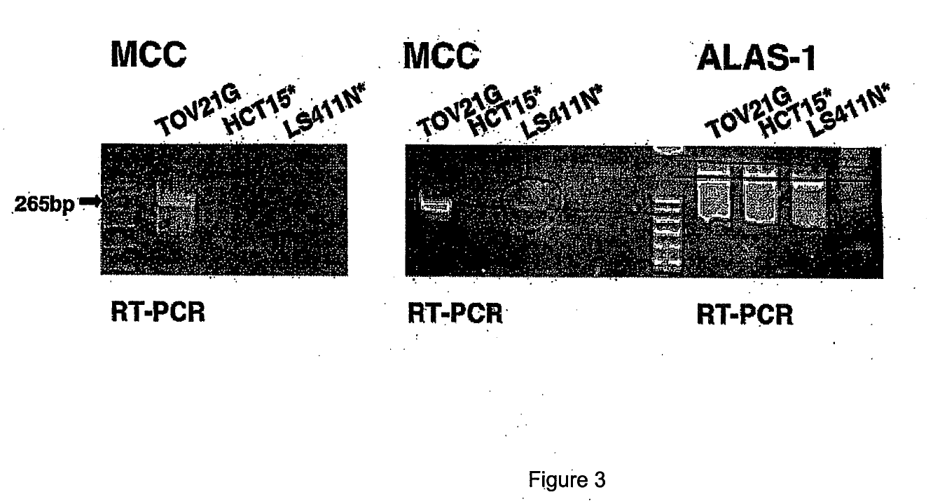

To correlate our sequencing results with the MCC gene expression, cDNA was prepared from three cell lines HCT15, LS411N and TOV21G. RT-PCR was carried out using a primer set in exons 1 and 2. The predicted 265-bp fragment was consistently amplified in the cell line TOV21G that showed no methylation of the MCC promoter (FIG. 3). No fragment was detectable in the cell line HCT15, whereas the control housekeeping gene ALAS-1 amplified normally. A MCC cDNA fragment was detectable in the cell line LS411N but with much lower intensity compared with the control ALAS-1 PCR and it was not always detectable in every RT-PCR. Significantly, the 26 CpG sites in region −304 to −139 were the shown to be an important indicator of MCC gene silencing or reduced expression.

Methylation-specific PCR.

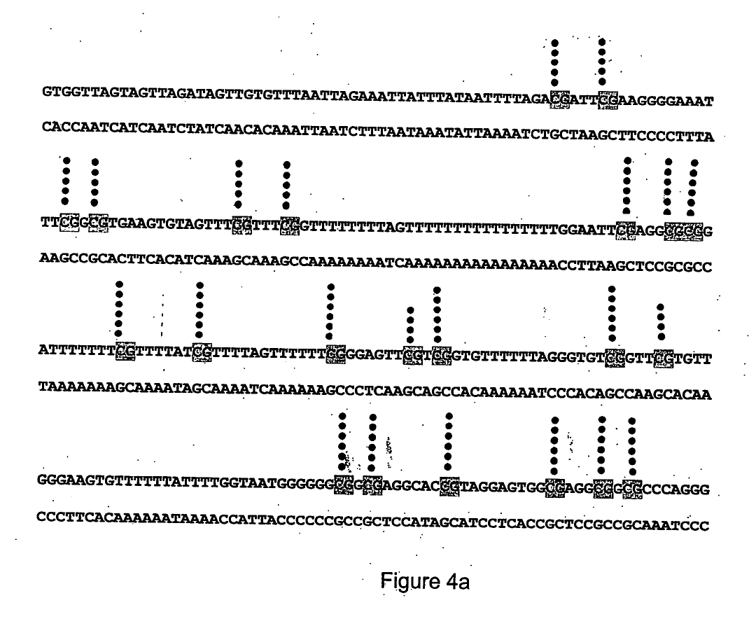

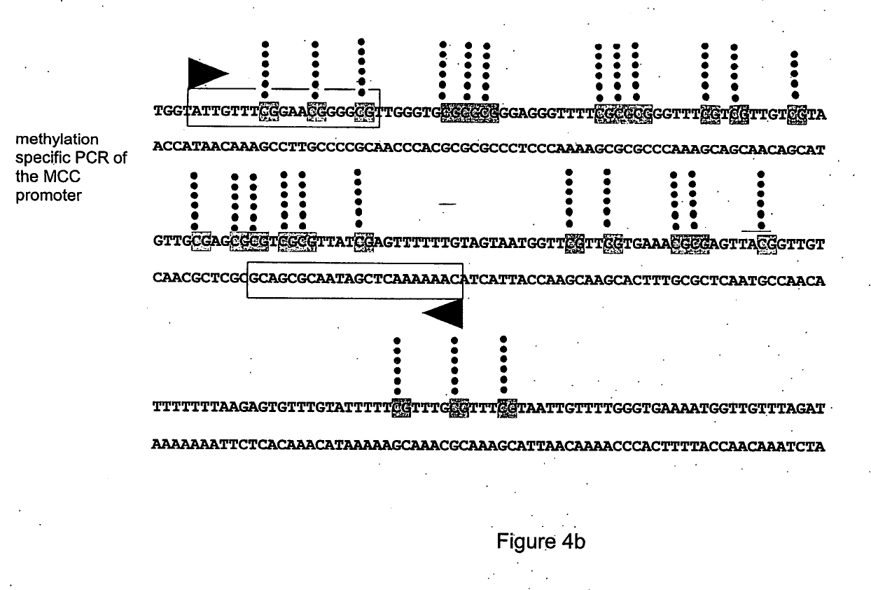

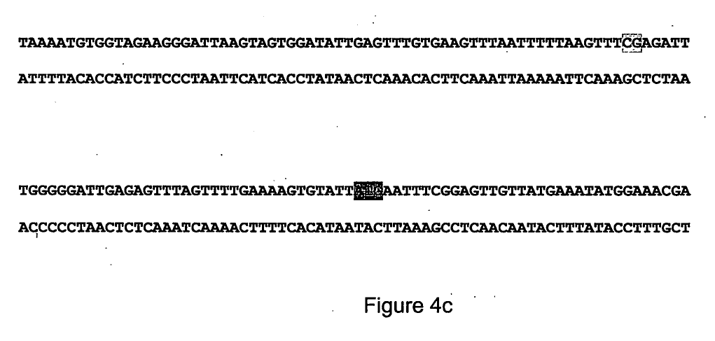

Proceeding on the basis of data correlating reduced MCC gene expression with methylation in the region between −304 to −139 of the promoter, we designed a methylation-specific PCR assay to amplify the region of the MCC gene promoter between about position −304 to −139 (FIG. 4). The reason for selecting this area were twofold. First, it was advantageous to avoid the region of unconverted DNA that would produce false positives for methylation. Second, although it was possible to detect methylation in the region of variable methylation, it might produce false negatives and miss those specimens with greatly reduced expression.

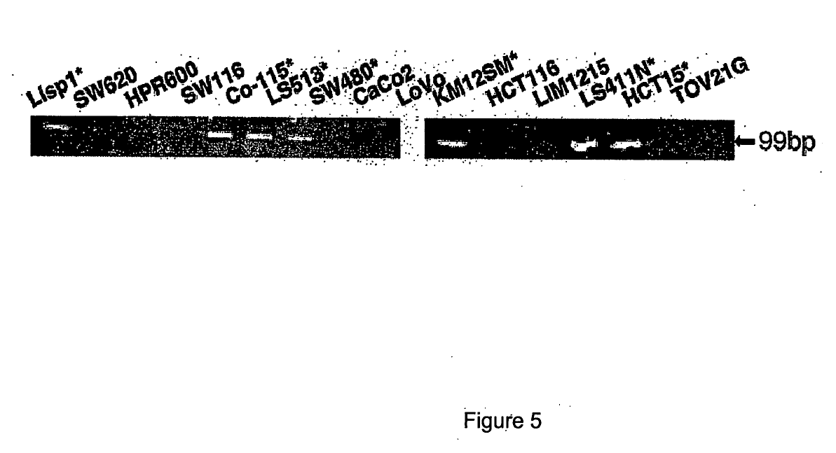

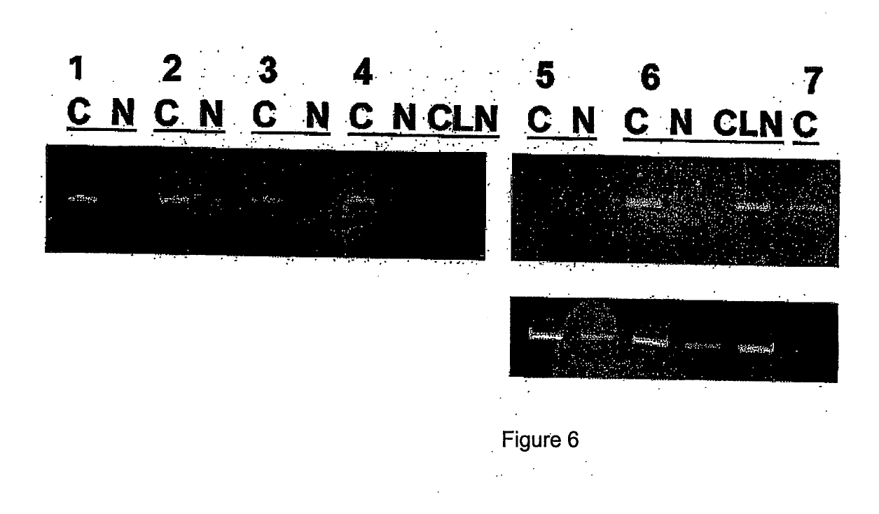

Two primers specific for the methylated sequence that contained 3 and 4 CpG sites (i.e., SEQ ID Nos: 18 and 20) were used to amplify the MCC promoter in the 14 cell lines for which bisulphite sequencing results were available. Only the six cell lines that had showed methylation produced the predicted 99bp fragment (FIG. 5). In contrast, no amplification product was detected in the eight unmethylated cell lines. The integrity of the bisulphite-treated DNA specimens was confirmed using primers for the MYOD1 gene, which amplify independently of CpG methylation (data not shown). We next proceeded to amplify the MCC promoter from bisulphite-treated clinical colon cancer specimens using the same protocol and MYOD1 as the internal control. The 99-bp fragment was detected in 54 out of 103 (52.4%) of stage C colon cancers but was not detected in the corresponding normal tissue from the same patients (FIG. 6).

Correspondence between promoter methylation and lack of RNA expression of the MCC gene was further confirmed using methylation specific PCR and RT-PCR in colorectal cancer cell lines Lisp1 and KM12SM. Correspondence between non-methylation and normal RNA expression of the MCC gene was confirmed in colorectal cancer cell lines SW620, LoVo, HCT116, and LIM1215.

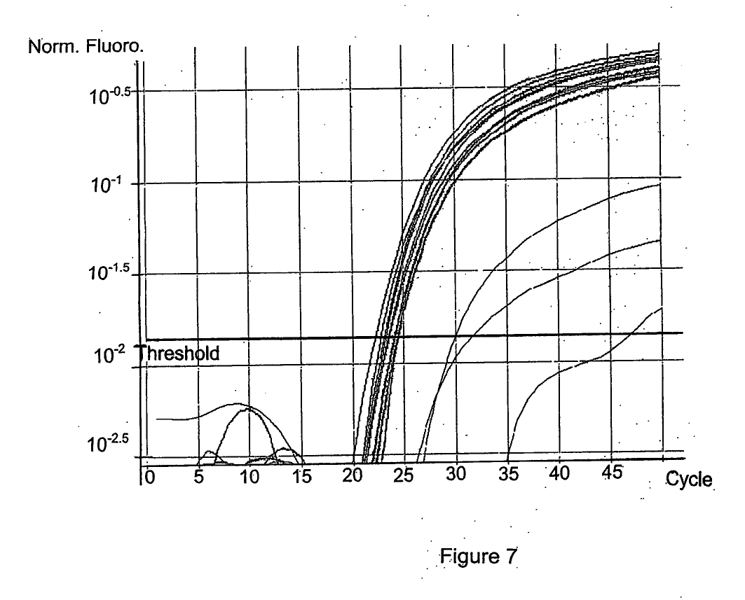

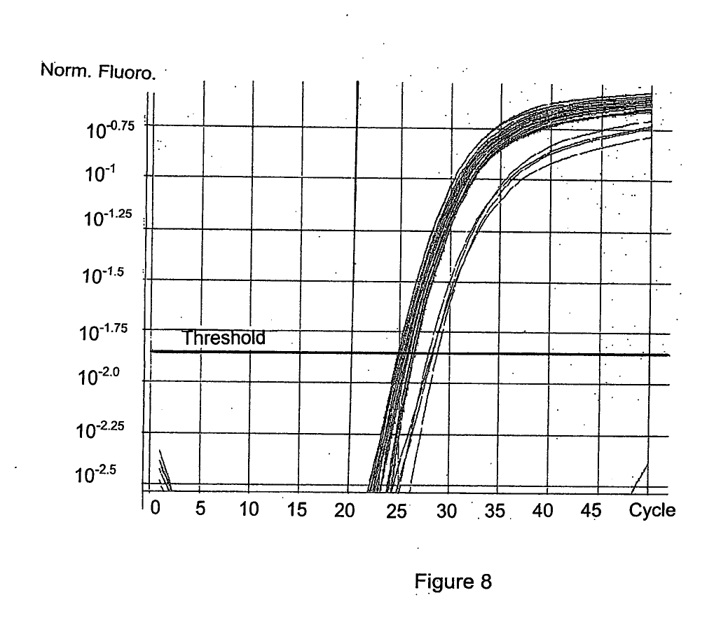

Data presented in FIGS. 7 and 8 show the results of Real Time PCR (TaqMan assay). Methylation of p16^{ARF14} was also determined essentially as described herein for the cohort of 103 patients having Stage C colorectal cancer, to determine if there is simultaneous methylation of both gene promoters. Data are summarized in Table 1. Those data indicate that MCC methylation is associated with p16^{ARF14} methylation (p=0.00012).

TABLE 1
Number of patients with MCC and p16
methylation in stage C colon cancer

| | | | Total no. |
|---|---|---|---|
| | p16 methylated | p16 unmethylated | patients |
| MCC methylated | 27 | 27 | 54 (52.4%) |
| MCC unmethylated | 7 | 42 | 49 (47.6%) |
| Total no. patients | 34 (33.0%) | 69 (67.0%) | 103 |

### EXAMPLE 2

### Methylation of MCC Promoter Sequences in Hyperplastic Polyps

Tissue Samples

A total of 43 polyp samples were obtained. At the filing date, 19 samples had been classified as hyperplastic polyp or adenoma Seven hyperplastic polyps were analyzed, at least two of which were classified as variant BPs. Twelve adenomas were also analyzed.

Methylation Specific Real-time PCR.

The primers MCC-metF2 (SEQ ID NO: 19) and MCC-metR2 (SEQ ID NO: 23) were used in a real-time PCR together with the methylated amplicon-specific fluorogenic hybridisation probe 5′-6FAM-CGCGTCGCGTrATCGAGTT-BHQ-1-3′ (SEQ ID NO: 22). The PCR was carried out using the RealMasterMix Probe ROX (Eppendorf) with 4 mM Mg²⁺ in a Rotorgene 3000 (Corbett Research). PCR conditions were 95° C. (15s), 60° C. (60s) for 50 cycles, with an initial denaturation at 95° C. for 2 min. All

specimens were also amplified with the primers and a TaqMan probe (Usadel et al, Cancer Res 62, 371-375, 2002) specific for the MYOD gene to ensure that each specimen had been bisulphite converted regardless of methylation status. The same PCR conditions were used as for MCC.

APC gene promoter methylation and p16 gene promoter methylation were also analysed in these specimens. The p16 gene promoter was amplified using primers for the methylated sequences and PCR conditions as described herein above (e.g., Example 1 and Herman et al., Proc. Natl acad. Sci. USA 93, 9821-9826, 1996). Methylated APC gene promoter sequences were amplified using the PCR primers and fluorogenic hybridization TaqMan probe as described by Usadel et al., Cancer Res. 62, 371-175, 2002). Real-time PCRs were carried out using the real MasterMix Probe ROX (Eppendorf) in a Rotorgene 3000 (Corbett Research). PCR conditions were 95° C. (15s), 60° C. (60s) for 50 cycles, with an initial denaturation at 95° C. for 2 min.

Results

Data for MCC are summarized in Table 2. All of the HP analysed and about 40% of adenomas showed MCC methylation. Those data indicate that MCC methylation is associated with the early stages of colorectal tumorigenesis and is more common in hyperplastic polyps than in adenoma (p=0.016).

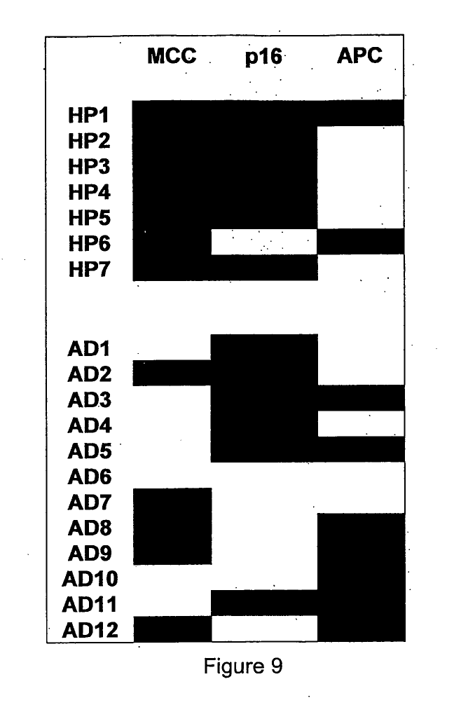

The MCC, APC and p16 methylation data are summarized in FIG. 9. Those data show that methylation of MCC was found with either p16 methylation and/or APC methylation in all HPs examined, and that methylation of MCC or pl6 or APC occurred in 11 of the 12 adenomas tested.

TABLE 2
MCC methylation in early stage of colon carcinogenesis

| | Hyperplastic polyps | Adenomas |
|---|---|---|
| MCC methylated | 7 | 5 |
| MCC unmethylated | 0 | 7 |
| Total no. specimens | 7 | 12 |

1. A method of diagnosing colorectal cancer or hyperplastic polyposis in a subject, said method comprising determining the degree of methylation of nucleic acid that regulates expression of an MCC gene in a sample derived from the subject, wherein an enhanced degree of methylation in the sample relative to a suitable control sample is indicative of colorectal cancer or hyperplastic polyposis.

2. A method for determining a predisposition for neoplastic transformation in a subject having colorectal lesions or polyps said method comprising determining the degree of methylation of nucleic acid that regulates expression of an MCC gene in a sample derived from a lesion or polyp wherein an enhanced degree of methylation in the sample relative to a suitable control sample is indicative of a predisposition for neoplastic transformation in the subject.

3. A method of monitoring the efficacy of treatment for colorectal cancer or hyperplastic polyposis in a subject, said method comprising treating a subject in need of treatment for colorectal cancer or hyperplastic polyposis and determining the degree of methylation of nucleic acid that regulates expression of an MCC gene in a sample derived from the subject, wherein a degree of methylation in the sample comparable to that for a suitable control sample is indicative of effective treatment.

4. A method of monitoring the efficacy of treatment for colorectal cancer or hyperplastic polyposis in a subject, said method comprising treating a subject in need of treatment for colorectal cancer or hyperplastic polyposis and determining the degree of methylation of nucleic acid that regulates expression of an MCC gene in a sample derived from the subject, wherein a hyper methylation of the MCC promoter in the sample compared to the level of methylation for a suitable control sample indicates that treatment is not effective.

5. The method according to claim 1 wherein the colorectal cancer is a carcinoma in situ.

6. The method of claim 5 wherein the carcinoma in situ is characterized by the presence of polyps in the proximal and distal colorectum.

7. The method of claim 6 wherein the polyps are hyperplastic polyps.

8. The method according to claim 1 wherein the colorectal cancer is a stage A colorectal cancer.

9. The method according to claim 1 wherein the colorectal cancer is a stage B colorectal cancer.

10. The method according to claim 1 wherein the colorectal cancer is a stage C colorectal cancer.

11. The method according to claim 1 wherein the colorectal cancer is a stage D colorectal cancer.

12. The method according to claim 1 wherein the sample comprises tissue or cells from the colorectum, colorectal polyps, or a stool sample.

13. The method according to claim 1 wherein determining the degree of methylation of nucleic acid that regulates expression of an MCC gene in a sample consists of determining the degree of methylation in a region of an MCC gene consisting of nucleotides 1 to 560 of SEQ ID NO: 3 or a complementary sequence thereto.

14. The method according to claim 1 wherein determining the degree of methylation of nucleic acid that regulates expression of an MCC gene in a sample consists of determining the degree of methylation in a region of an MCC gene consisting of nucleotides 292 to 458 of SEQ ID NO: 3 or a complementary sequence thereto.

15. The method according to claim 1 wherein determining the degree of methylation of nucleic acid that regulates expression of an MCC gene in a sample consists of determining the degree of methylation in a region of an MCC gene consisting of nucleotides 284 to 304 of SEQ ID NO: 3 or a complementary sequence thereto.

16. The method according to claim 1 wherein determining the degree of methylation of nucleic acid that regulates expression of an MCC gene in a sample consists of determining the degree of methylation in a region of an MCC gene consisting of nucleotides 335 to 355 of SEQ ID NO: 3 or a complementary sequence thereto.

17. The method according to claim 1 wherein determining the degree of methylation of nucleic acid that regulates expression of an MCC gene in a sample consists of determining the degree of methylation in a region of an MCC gene consisting of nucleotides 361 to 404 of SEQ ID NO: 3 or a complementary sequence thereto.

18. The method according to claim 1 comprising performing methylation-sensitive endonuclease digestion of DNA from the sample.

19. The method according to claim 1 comprising hybridizing a probe comprising an MspI/HpaII recognition site to nucleic acid digested with HpaII enzyme for a time and under conditions sufficient to digest non-methylated DNA.

20. The method of claim 19 wherein the probe comprises nucleotides from about position 291 to about position 294 of SEQ ID NO: 3 or a complementary sequence thereto.

21. The method according to claim 1 comprising subjecting nucleic acid from the sample to DNase I digestion and amplifying the fragments produced using primers that bind to or flank methylated regions in the MCC gene promoter comprising a sequence from position 1 to about position 560 of SEQ ID NO: 3 or a complementary sequence thereto.

22. The method of claim 21 wherein the primers flank a region of the MCC gene including residues 284-404 of SEQ ID NO: 3 or a complementary sequence thereto.

23. The method of claim 21 wherein a primer comprises a nucleotide sequence selected from the group consisting of SEQ ID NOs: 11-16 or 18-23 or 25-34 or a complementary sequence thereto.

24. The method according to claim 21 further comprising detecting the amplified fragments with a nucleic acid probe capable of specifically hybridizing to the amplified fragment.

25. The method of claim 24 wherein the probe comprises a sequence set forth in SEQ ID NO: 12 or 15 labelled at its 5′-end and 3′-end with one or more fluorescent ligands and/or a Black Hole Quencher (BHQ).

26. The method of claim 25 wherein the fluorescent ligands are selected from the group consisting of TAMRA and FAM.

27. The method according to claim 1 comprising treating nucleic acid from the sample with an amount of a compound that selectively mutates nonmethylated cytosine residues in nucleic acid under conditions sufficient to induce mutagenesis.

28. The method of claim 27 wherein the compound is a metal salt of bisulphite.

29. The method of claim 28 wherein the compound is sodium bisulphite or potassium bisulphite.

30. The method according to claim 27 further comprising amplifying a fragment using primers that flank a methylated cytosine residue or mutated residue at an equivalent position in non-methylated nucleic acid.

31. The method of claim 30 wherein the primers flank a region of the MCC gene including residues 284-404 of SEQ ID NO: 3 or a complementary sequence thereto.

32. The method of claim 30 wherein a primer comprises a nucleotide sequence selected from the group consisting of SEQ ID NOs: 11-16 or 18-23 or 25-34 or a complementary sequence thereto.

33. The method according to claim 30 further comprising detecting the amplified fragments with a nucleic acid probe capable of specifically hybridizing to the amplified fragment.

34. The method of claim 33 wherein the probe comprises a sequence set forth in SEQ ID NO: 12 or 15 labelled at its 5′-end and 3′-end with one or more fluorescent ligands and/or a Black Hole Quencher (BHQ).

35. The method of claim 34 wherein the fluorescent ligands are selected from the group consisting of TAMRA and FAM.

36. The method according to claim 1 wherein the enhanced degree of methylation consists of at least about 3 CpG islands in the nucleic acid being methylated in the sample relative to a suitable control sample.

37. The method according to claim 1 further comprising determining the degree of methylation of a control sample.

38. The method according to claim 1 wherein a control sample is a data set selected from the group consisting of:
a) a data set comprising measurements of the degree of methylation for a typical population of subjects known to have colorectal cancer;
b) a data set comprising measurements of the degree of methylation for the subject being tested wherein said measurements have been made previously, such as, for example, when the subject was known to healthy or, in the case of a subject having colorectal cancer, when the subject was diagnosed or at an earlier stage in disease progression;
c) a data set comprising measurements of the degree of methylation for a healthy individual or a population of healthy individuals;
d) a data set comprising measurements of the degree of methylation for a normal individual or a population of normal individuals; and
e) a data set comprising measurements of the degree of methylation from the subject being tested wherein the measurements are determined in a matched sample.

39. The method according to claim 1 further comprising isolating the sample from the subject.

40. The method of claim 39 wherein the sample is derived by colonoscopy.

41. The method according to claim 1 further comprising determining the level of methylation another gene selected from the group consisting of HLTF, APC, p16^{INK4a},p14^{ARF}, HPP1, hMLH1, MGMT, and combinations thereof wherein hypermethylation of MCC and the other gene is indicative of colorectal cancer or hyperplastic polyposis or a predisposition of a lesion or polyp to neoplastic transformation.

42. The method according to claim 1 further comprising determining a CpG island methylation phenotype (CIMP) for the subject wherein methylation of at least about 3-4 CIMP markers in combination with hypermethylation of MCC is indicative of colorectal cancer or hyperplastic polyposis or a predisposition of a lesion or polyp to neoplastic transformation.

43. The method according to claim 1 further comprising determining a mutation in the APC gene or DCC gene wherein hypermethylation of MCC in combination with a deletion or point mutation of APC or DCC are indicative of of colorectal cancer or hyperplastic polyposis or a predisposition of a lesion or polyp to neoplastic transformation.

44. An isolated nucleic acid probe or primer that is capable of selectively hybridising to a region of the MCC gene promoter that is hyper methylated in a colorectal cancer, wherein said region comprises or is contained within nucleotide residues from about position 284 to about position 404 of SEQ ID NO: 3 or SEQ ID NO: 17 or SEQ ID NO: 24 or a complementary sequence thereto.

45. A probe or primer consisting of a nucleotide sequence set forth in any one SEQ ID NOs: 11-16 or 18-23 or 25-34 or a complementary sequence thereto.


SEQUENCE LISTING (46 sequences)

SEQ ID NO: 1 - DNA, 4181 bp, Homo sapiens
  CDS (221)..(2707)
cctcctgcagcaatggctcgtccgtgaaacgcgagccacggctgctctttttaagagtgcctgcatcctccgtttgcgcttcgcaactgtcctgggtgaaaatggctgtctagactaaaatgtggcagaagggaccaagcagtggatattgagcctgtgaagtccaactcttaagctccgagacctgggggactgagagcccagctctgaaaagtgcatcatgaattccggagttgccatgaaatatggaaacgactcctcggccgagctgagtgagctccattcagcagccctggcatcactaaagggagatatagtggaacttaataaacgtctccagcaaacagagagggaacgggaccttctggaaaagaaattggccaaggcacagtgcgagcagtcccacctcatgagagagcatgaggatgtccaggagcgaacgacgcttcgctatgaggaacgcatcacagagctccacagcgtcattgcggagctcaacaagaagatagaccgtctgcaaggcaccaccatcagggaggaagatgagtactcagaactgcgatcagaactcagccagagccaacacgaggtcaacgaggactctcgaagcatggaccaagaccagacctctgtctctatccccgaaaaccagtctaccatggttactgctgacatggacaactgcagtgacctgaactcagaactgcagagggtgctgacagggctggagaatgttgtctgcggcaggaagaagagcagctgcagcctctccgtggccgaggtggacaggcacattgagcagctcaccacagccagcgagcactgtgacctggctattaagacagtcgaggagattgagggggtgcttggccgggacctgtatcccaacctggctgaagagaggtctcggtgggagaaggagctggctgggctgagggaagagaatgagagcctgactgccatgctgtgcagcaaagaggaagaactgaaccggactaaggccaccatgaatgccatccgggaagagcgggaccggctccggaggcgggtcagagagcttcaaactcgactacagagcgtgcaggccacaggtccctccagccctggccgcctcacttccaccaaccgcccgattaaccccagcactggggagctgagcacaagcagcagcagcaatgacattcccatcgccaagattgctgagagggtgaagctatcaaagacaaggtccgaatcgtcatcatctgatcggccagtcctgggctcagaaatcagtagcataggggtatccagcagtgtggctgaacacctggcccactcacttcaggactgctccaatatccaagagattttccaaacactctactcacacggatctgccatctcagaaagcaagattagagagtttgaggtggaaacagaacggctgaatagccggattgagcacctcaaatcccaaaatgacctcctgaccataaccttggaggaatgtaaaagcaatgctgagaggatgagcatgctggtgggaaaatacgaatccaatgccacagcgctgaggctggccttgcagtacagcgagcagtgcatcgaagcctacgaactcctcctggcgctggcagagagtgagcagagcctcatcctggggcagttccgagcggcgggcgtggggtcctcccctggagaccagtcgggggatgaaaacatcactcagatgctcaagcgagctcatgactgccggaagacagctgagaacgctgccaaggccctgctcatgaagctggacggcagctgtgggggagcctttgccgtggccggctgcagcgtgcagccctgggagagcctttcctccaacagccacaccagcacaaccagctccacagccagtagttgcgacaccgagttcactaaagaagacgagcagaggctgaaggattatatccagcagctcaagaatgacagggctgcggtcaagctgaccatgctggagctggaaagcatccacatcgatcctctcagctatgacgtcaagcctcggggagacagccagaggctggatctggaaaacgcagtgcttatgcaggagctcatggccatgaaggaggagatggccgagttgaaggcccagctctacctactggagaaagagaagaaggccctggagctgaagctgagcacgcgggaggcccaggagcaggcctacctggtgcacattgagcacctgaagtccgaggtggaggagcagaaggagcagcggatgcgatccctcagctccaccagcagcggcagcaaagataaacctggcaaggagtgtgctgatgctgcctccccagctctgtccctagctgaactcaggacaacgtgcagcgagaatgagctggctgcggagttcaccaacgccattcgtcgagaaaagaagttgaaggccagagttcaagagctggtgagtgccttggagagactcaccaagagcagtgaaatccgacatcagcaatctgcagagttcgtgaatgatctaaagcgggccaacagcaacctggtggctgcctatgagaaagcaaagaaaaagcatcaaaacaaactgaagaagttagagtcgcagatgatggccatggtggagagacatgagacccaagtgaggatgctcaagcaaagaatagctctgctagaggaggagaactccaggccacacaccaatgaaacttcgctttaatcagcactcacgcaccggagttctgcccatgggaagtaaactgcagcaggccactggggacagaagggcccatgtacttgttgggaggaggaggaaagggaaggctggcaggtaggtcggcacttggacaatggagtgccccaactcaacccttggggtgactggccatggtgacattgtggactgtatccagaggtgcccgctcttccctcctgggcccacaacagcgtgtaaacacatgttctgtgcctgctcagcagagcctcgtttctgctttcagcactcactctccccctcctcttctggtctggcggctgtgcatcagtgggatcccagacatttgtttctgtaagattttccattgtatcctctttttggtagatgctgggctcatcttctagaatctcgtttctcctctttcctcctgcttcatgggaaaacagacctgtgtgtgcctccagcatttaaaaggactgctgatttgtttactacagcaaggctttggtttccaagtcccgggtctcaactttaagatagaggcggccataagaggtgatctctgggagttataggtcatgggaagagcgtagacaggtgttacttacagtcccagatacactaaagttacaaacagaccaccaccaggactgtgcctgaacaattttgtattgagagaataaaaacttccttcaatcttcattttggaggcagggctgggaagggagcgctctcttgattctgggatttctccctctcagtggagccttattaatatccaagacttagagctgggaatctttttgatacctgtagtggaactaaaattctgtcaggggtttcttcaagagctgagaaacattattagcacttcccgccccagggcactacataattgctgttctgctgaatcaaatctcttccacatgggtgcatttgtagctctggacctgtctctacctaaggacaagacactgaggagatactgaacattttgcaaaacttatcacgcctacttaagagtgctgtgtaacccccagttcaagacttagctcctgttgtcatgacggggacagagtgagggaatggtagttaaggcttcttttttgcccccagatacatggtgatggttagcatatggtgcttaaaaggttaaatttcaagcaaaatgcttacagggctaggcagtaccaaagtaactgaattatttcaggaaggtcttcaatcttaaaacaaattcattattctttttcagttttacctcttctctctcagttctacactgatacacttgaaggaccatttactgtttttttctgtagcaccagagaatccatccaaagttccctatgaaaaatgtgttccattgccatagctgactacaaattaaagttgaggaggtttctgcatagagtctttatgtccataagctacgggtaggtctattttcagagcatgatacaaattccacagg

SEQ ID NO: 2 - protein, 829 aa, Homo sapiens
MNSGVAMKYGNDSSAELSELHSAALASLKGDIVELNKRLQQTERERDLLEKKLAKAQCEQSHLMREHEDVQERTTLRYEERITELHSVIAELNKKIDRLQGTTIREEDEYSELRSELSQSQHEVNEDSRSMDQDQTSVSIPENQSTMVTADMDNCSDLNSELQRVLTGLENVVCGRKKSSCSLSVAEVDRHIEQLTTASEHCDLAIKTVEEIEGVLGRDLYPNLAEERSRWEKELAGLREENESLTAMLCSKEEELNRTKATMNAIREERDRLRRRVRELQTRLQSVQATGPSSPGRLTSTNRPINPSTGELSTSSSSNDIPIAKIAERVKLSKTRSESSSSDRPVLGSEISSIGVSSSVAEHLAHSLQDCSNIQEIFQTLYSHGSAISESKIREFEVETERLNSRIEHLKSQNDLLTITLEECKSNAERMSMLVGKYESNATALRLALQYSEQCIEAYELLLALAESEQSLILGQFRAAGVGSSPGDQSGDENITQMLKRAHDCRKTAENAAKALLMKLDGSCGGAFAVAGCSVQPWESLSSNSHTSTTSSTASSCDTEFTKEDEQRLKDYIQQLKNDRAAVKLTMLELESIHIDPLSYDVKPRGDSQRLDLENAVLMQELMAMKEEMAELKAQLYLLEKEKKALELKLSTREAQEQAYLVHIEHLKSEVEEQKEQRMRSLSSTSSGSKDKPGKECADAASPALSLAELRTTCSENELAAEFTNAIRREKKLKARVQELVSALERLTKSSEIRHQQSAEFVNDLKRANSNLVAAYEKAKKKHQNKLKKLESQMMAMVERHETQVRMLKQRIALLEEENSRPHTNETSL

SEQ ID NO: 3 - DNA, 595 bp, Homo sapiens
gtggccagcagctagacagctgtgttcaatcagaaaccacttacaactccagacgattcgaaggggaaacttcggcgtgaagtgcagctccgctccggctccctctagctttctttccttctctggaatccgaggcgcggatcttcctcgccccaccgccctagttttttcgggagctcgccggtgccctctagggtgtcggctcgtgctgggaagtgccctccatcctggtaatggggggcggcgaggcaccgtaggagtggcgaggcggcgcccagggtggcactgccccggaacggggcgctgggtgcgcgcgggagggtccccgcgcgggctccgccgctgccgcagctgcgagcgcgccgcgccaccgagcctcctgcagcaatggctcgtccgtgaaacgcgagccacggctgctctttttaagagtgcctgcatcctccgtttgcgcttcgcaactgtcctgggtgaaaatggctgtctagactaaaatgtggcagaagggaccaagcagtggatattgagcctgtgaagtccaactcttaagctccgagacctgggggactgagagcccagctctgaaaagtgcatc

SEQ ID NO: 4 - DNA, 5457 bp, Homo sapiens
  CDS (313)..(3339)
ttcgcctctctcatgatcccaggaagactaacaggctctttgtgacactggcactgcgctgtcggatccagctgttgtcattgtgaaggggtgttctcttcctcatttcctgaaggatttgttgcagaaggagacggcgtcgacgtctgactggactcgcggcgacttacctttcagtcgtgcgctcctgatccggcgctcggaatttgtccccggcttcagggctgcggggcctggaaggaggcgtatcgaggcggctcgaaaacgatccaggggagccgaggcgctcctcttgtcatcccactcagcgccatgtcctggatgttcaagagggatccagtttggaagtacttgcagactgtccagtatggagttcatggaaattttccacgcctctcatatccaactttctttctacgttttgaattccaagatgttatccctccagatgactttctaactagtgatgaagaagtagattccgttttatttggaagtttgagaggtcatgtggttggactacgctattacacgggagtagttaataataatgaaatggttgcattacaacgagatcctaataacccttatgataagaatgcaattaaagtaaacaatgtgaatggaaatcaagttggccatttaaagaaagagcttgcaggtgctttggcctatatcatggacaacaaattggcacaaattgaaggggtagttccttttggtgcaaacaatgcttttaccatgcctctgcatatgactttttggggaaaagaagaaaatagaaaagcggtttcagatcagttgaagaaacatggatttaaattgggtcctgcaccaaaaactttaggattcaatttggaaagtggttggggctctggaagagctggaccaagctatagtatgccagtgcatgctgcagtacagatgacaactgaacagcttaaaacagaatttgacaaattgtttgaagatttaaaagaagatgataaaacccatgaaatggaaccagctgaggctattgaaacaccactgcttccacatcaaaaacaagctctagcttggatggtgtcacgggaaaatagcaaagaacttccaccattctgggaacagcgaaatgacttatactataacacaataacaaatttttctgagaaggaccgaccagaaaatgtccatggaggaattttagctgatgatatgggtttgggtaaaactcttacggccattgcagtaatccttaccaacttccatgatggcagacctcttcctattgaaagagttaaaaagaatctactgaagaaggaatataatgttaacgatggctctatgaaacttggaggaaacaataccagtgaaaaggcagatggactaagcaaagacgcatctagatgtagtgaacaacccagtatttcagatatcaaggagaagagtaagtttcgcatgtcagaattgtctagctcccgccccaaaagaagaaaaactgctgtccagtacatagaaagcagtgattcagaggaaattgaaacaagtgaattgccgcagaaaatgaaaggcaaactgaaaaatgtacagtctgaaactaaaggcagggcgaaagcaggatcttctaaggttatagaagatgtggcatttgcatgtgcattaacttcatctgttcctacaacaaaaaagaaaatgttgaaaaagggagcttgtgcagtggaggggtcaaagaaaactgatgttgaggagagaccaagaacaacactgatcatctgtccgctttctgtgttaagcaactggattgaccagtttggacaacatataaaatcagatgtacacttgaatttttatgtttattatggtcctgatcgtattagagaaccggccttactttcaaaacaggatattgttttgactacgtataatattttaactcatgactatggaactaaaggagatagtccattacatagcataaggtggctaagagtgatcctggatgaaggacatgccatacgaaatccaaatgctcagcagacaaaagctgtacttgacttagaatcagaaagaagatgggttttgacaggtactccaatccagaattctttaaaggacttgtggtctcttctttcctttttaaaacttaaaccatttattgatagagaatggtggcatagaacaatacagcgtcctgtcacaatgggagatgaaggaggacttaggcgtttacagtccctaattaaaaatattacacttagaagaacaaagacaagcaaaattaaaggaaaacctgttttggagttaccagaacgtaaagtatttattcagcacattacactttcagatgaagagagaaagatttatcagtctgtgaaaaatgaaggcagagccactattggaaggtattttaatgaagggactgtcctggcacattatgcagatgtcctgggtcttttgcttagactgcggcaaatttgttgccatacttaccttcttacaaatgcagtgtcttccaatggcccctcaggaaatgatacacctgaagaactgagaaagaagttaataaggaagatgaagttaattctgagctcaggttcagatgaggaatgtgcaatttgcctggattctttaacagttcctgtgataacacattgtgcacatgtattttgtaaaccctgtatttgccaagtcattcagaatgagcagccacatgctaaatgccctttatgcagaaatgatatacatgaagataatttattagaatgtcctccagaagaattagcacgtgacagtgagaaaaagtctgatatggaatggacatccagttcaaagattaatgcgctaatgcacgcattgactgacttaagaaagaagaatcccaacataaaaagtttggttgtttctcagtttacaacattcctgtctttaatagaaataccacttaaagcctctggatttgtgtttactcgtttggatggttccatggcccaaaagaaaagagttgaatcaattcagtgttttcaaaacactgaagcaggatctccaactataatgcttctgtccttaagagcaggtggagttggtttgaatctgtctgcagcttctcgagtgtttttaatggatccagcctggaatcctgctgctgaagatcagtgctttgacagatgccatagacttggtcagaagcaagaagttatcatcacaaaattcattgtaaaggactctgttgaagaaaatatgctgaaaatacaaaacaaaaagagagaacttgcagcaggagcctttggaactaaaaaaccaaatgctgacgaaatgaaacaagccaaaattaatgaaatcagaacattaattgacttataatttgtgggattttagtaaggtcagtttgattggatacttaagttttagaaatgagaaaaatacagagttttagaaatgagatctagagaacacgtcttctaaaaggggcatattttatattagtgaagaggtattactgacacaatttcttctatatatgaacctatttttaatgaaacttcaaatagcaataagttccgttatatactgtggcctgaaataatttgagaaaaaaggttactttgttattcagcttttcataatatctatgctgagtattttcacgtatcttccaagtactcagctttttcgtatttcaaataaggtcagaccttttatacttttgaccaaatagttaatttctatgttggacacttagttatttaccaaagcctccgatttgtgatgcagtgtttgtagtccttgtaaacaatatatacagactatacaagaattaattttattgggctttcaaaaaccatatttgcattccagaaccaaatcttaaatgagaccaaagtccaggttagcacaggtttttatttttcctacagctacattgagatatagttcacataaatgacttgggagttttatgttcatgaaaaaattagggattatgttaagagtactattttttccattttagtaaagtagtactacacacacattgtttaaatgtaactgctcgtgtctgaggtataaatatagtctgtgggagtgagagcaaaccagtcctacccaatttgatttgaatattttaaattatggaacctgctaaatagatatttctataaatagataatttttatttatgtagctttttttggaagtaactttataaatttttataattcagaagactactatatgtgagaggcgtgatatctggatggaagttgggctggatgatctccaaagtcgtttcaactcttaaagacatcttaatcctgaatgtaaacaattgttatgtgtttagaatcagaatttgattttgaacttgagtaattcatccttacagctatctgtagaattagtcatcttttttcttttcttttttttttacttttttgttaataagcaaacttatttgctgcagaatttgggttgactcctgagcatatttaaaacaaagaagctagaaatttagcagtcagattaggtaggtggttttatttcaaagggaaactttaatccaaagaaagattaattactctaacaaacaagagaagcttcatgtttgatgatacagatttaagaatacctgagacttaagagtgttggaagtcatttgatagaaaagatgagatcggagacaatgttgtgttatagggcacacattgaaggtatatgccaaatctctcacaagataagtccttttctccctgtgccgttttcttgctcaaagggaacagtgaattagccagctagaatcttcctggtccctttttgaggcagtagcaggtaaggaaagggctgattttcatcaaaaccaagacctttctgcaggcatgatagtggaataataatgtgggattagcccgctagcattaggagcagttgggaagttacctggtagatcaagcattacacacaaaaaatcaagttgatcagagtatgggttctgcatgtagcaatacttcagtgagattaagtataaacagtttttggcaaaaaacaacacagtctactctttctgcttacaaagacaaagccttacaaactcactatgaaggtgaaagggagacagcttgcttctttgcccagacatttacaaagttgtttttaaaacacactcataagtaagtttggcaagttgtttaaaaaatgtctctttgttttgtacagttctgttagatgttgttatattttaaaagtttaatttaaaaaatttaatttgtccttcctaagaaggataaatatataaaaaagccatctggaatgaaaacttcctatatgctagctgttgtcttattattatatagaaaaataactttagaaaaatattgaagacattgtattaccacttgtgattcaaacaattttgtggttaaaactggattttaaatttaaaaatcaataaaaatttcaaatgttaaaaaaaaaaaaaaaaaa

SEQ ID NO: 5 - protein, 1009 aa, Homo sapiens [sequence omitted]

SEQ ID NO: 6 - protein, 887 aa, Homo sapiens [sequence omitted]

SEQ ID NO: 7 - DNA, 4608 bp, Homo sapiens
  CDS (1)..(4341)
atggagaatagtcttagatgtgtttgggtacccaagctggcttttgtactcttcggagcttccttgctcagcgcgcatcttcaagtaaccggttttcaaattaaagctttcacagcactgcgcttcctctcagaaccttctgatgccgtcacaatgcggggaggaaatgtcctcctcgactgctccgcggagtccgaccgaggagttccagtgatcaagtggaagaaagatggcattcatctggccttgggaatggatgaaaggaagcagcaactttcaaatgggtctctgctgatacaaaacatacttcattccagacaccacaagccagatgagggactttaccaatgtgaggcatctttaggagattctggctcaattattagtcggacagcaaaagttgcagtagcaggaccactgaggttcctttcacagacagaatctgtcacagccttcatgggagacacagtgctactcaagtgtgaagtcattggggagcccatgccaacaatccactggcagaagaaccaacaagacctgactccaatcccaggtgactcccgagtggtggtcttgccctctggagcattgcagatcagccgactccaaccgggggacattggaatttaccgatgctcagctcgaaatccagccagctcaagaacaggaaatgaagcagaagtcagaattttatcagatccaggactgcatagacagctgtattttctgcaaagaccatccaatgtagtagccattgaaggaaaagatgctgtcctggaatgttgtgtttctggctatcctccaccaagttttacctggttacgaggcgaggaagtcatccaactcaggtctaaaaagtattctttattgggtggaagcaacttgcttatctccaatgtgacagatgatgacagtggaatgtatacctgtgttgtcacatataaaaatgagaatattagtgcctctgcagagctcacagtcttggttccgccatggtttttaaatcatccttccaacctgtatgcctatgaaagcatggatattgagtttgaatgtacagtctctggaaagcctgtgcccactgtgaattggatgaagaatggagatgtggtcattcctagtgattattttcagatagtgggaggaagcaacttacggatacttggggtggtgaagtcagatgaaggcttttatcaatgtgtggctgaaaatgaggctggaaatgcccagaccagtgcacagctcattgtccctaagcctgcaatcccaagctccagtgtcctcccttcggctcccagagatgtggtccctgtcttggtttccagccgatttgtccgtctcagctggcgcccacctgcagaagcgaaagggaacattcaaactttcacggtctttttctccagagaaggtgacaacagggaacgagcattgaatacaacacagcctgggtcccttcagctcactgtgggaaacctgaagccagaagccatgtacacctttcgagttgtggcttacaatgaatggggaccgggagagagttctcaacccatcaaggtggccacacagcctgagttgcaagttccagggccagtagaaaacctgcaagctgtatctacctcacctacctcaattcttattacctgggaaccccctgcctatgcaaacggtccagtccaaggttacagattgttctgcactgaggtgtccacaggaaaagaacagaatatagaggttgatggactatcttataaactggaaggcctgaaaaaattcaccgaatatagtcttcgattcttagcttataatcgctatggtccgggcgtctctactgatgatataacagtggttacactttctgacgtgccaagtgccccgcctcagaacgtctccctggaagtggtcaattcaagaagtatcaaagttagctggctgcctcctccatcaggaacacaaaatggatttattaccggctataaaattcgacacagaaagacgacccgcaggggtgagatggaaacactggagccaaacaacctctggtacctattcacaggactggagaaaggaagtcagtacagtttccaggtgtcagccatgacagtcaatggtactggaccaccttccaactggtatactgcagagactccagagaatgatctagatgaatctcaagttcctgatcaaccaagctctcttcatgtgaggccccagactaactgcatcatcatgagttggactcctcccttgaacccaaacatcgtggtgcgaggttatattatcggttatggcgttgggagcccttacgctgagacagtgcgtgtggacagcaagcagcgatattattccattgagaggttagagtcaagttcccattatgtaatctccctaaaagcttttaacaatgccggagaaggagttcctctttatgaaagtgccaccaccaggtctataaccgatcccactgacccagttgattattatcctttgcttgatgatttccccacctcggtcccagatctctccacccccatgctcccaccagtaggtgtacaggctgtggctcttacccatgatgctgtgagggtcagctgggcagacaactctgtccctaagaaccaaaagacgtctgaggtgcgactttacaccgtccggtggagaaccagcttttctgcaagtgcaaaatacaagtcagaagacacaacatctctaagttacacagcaacaggcctcaaaccaaacacaatgtatgaattctcggtcatggtaacaaaaaacagaaggtccagtacttggagcatgactgcacatgccaccacgtatgaagcagcccccacctctgctcccaaggactttacagtcattactagggaagggaagcctcgtgccgtcattgtgagttggcagcctcccttggaagccaatgggaaaattactgcttacatcttattttataccttggacaagaacatcccaattgatgactggattatggaaacaatcagtggtgataggcttactcatcaaatcatggatctcaaccttgatactatgtattactttcgaattcaagcacgaaattcaaaaggagtggggccactctctgatcccatcctcttcaggactctgaaagtggaacaccctgacaaaatggctaatgaccaaggtcgtcatggagatggaggttattggccagttgatactaatttgattgatagaagcaccctaaatgagccgccaattggacaaatgcaccccccgcatggcagtgtcactcctcagaagaacagcaacctgcttgtgatcattgtggtcaccgttggtgtcatcacagtgctggtagtggtcatcgtggctgtgatttgcacccgacgctcttcagcccagcagagaaagaaacgggccacccacagtgctggcaaaaggaagggcagccagaaggacctccgaccccctgatctttggatccatcatgaagaaatggagatgaaaaatattgaaaagccatctggcactgaccctgcaggaagggactctcccatccaaagttgccaagacctcacaccagtcagccacagccagtcagaaacccaactgggaagcaaaagcacctctcattcaggtcaagacactgaggaagcagggagctctatgtccactctggagaggtcgctggctgcacgccgagccccccgggccaagctcatgattcccatggatgcccagtccaacaatcctgctgtcgtgagcgccatcccggtgccaacgctagaaagtgcccagtacccaggaatcctcccgtctcccacctgtggatatccccacccgcagttcactctccggcctgtgccattcccaacactctcagtggaccgaggtttcggagcaggaagaagtcagtcagtgagtgaaggaccaactacccaacaaccacctatgctgcccccatctcagcctgagcattctagcagcgaggaggcaccaagcagaaccatccccacagcttgtgttcgaccaactcacccactccgcagctttgctaatcctttgctacctccaccaatgagtgcaatagaaccgaaagtcccttacacaccacttttgtctcagccagggcccactcttcctaagacccatgtgaaaacagcctcccttgggttggctggaaaagcaagatcccctttgcttcctgtgtctgtgccaacagcccctgaagtgtctgaggagagccacaaaccaacagaggattcagccaatgtgtatgaacaggatgatctgagtgaacaaatggcaagtttggaaggactcatgaagcagcttaatgccatcacaggctcagccttttaacatgtatttctgaatggatgaggtgaattttccgggaactttgcagcataccaattacccataaacagcacacctgtgtccaagaactctaaccagtgtacaggtcacccatcaggaccactcagttaaggaagatcctgaagcagttcagaaggaataagcattccttctttcacaggcatcaggaattgtcaaatgatgattatgagttccctaaacaaaagcaaagatgcattttcactgcaatgtcaaagtttagctgct

SEQ ID NO: 8 - protein, 1447 aa, Homo sapiens
MENSLRCVWVPKLAFVLFGASLLSAHLQVTGFQIKAFTALRFLSEPSDAVTMRGGNVLLDCSAESDRGVPVIKWKKDGIHLALGMDERKQQLSNGSLLIQNILHSRHHKPDEGLYQCEASLGDSGSIISRTAKVAVAGPLRFLSQTESVTAFMGDTVLLKCEVIGEPMPTIHWQKNQQDLTPIPGDSRVVVLPSGALQISRLQPGDIGIYRCSARNPASSRTGNEAEVRILSDPGLHRQLYFLQRPSNVVAIEGKDAVLECCVSGYPPPSFTWLRGEEVIQLRSKKYSLLGGSNLLISNVTDDDSGMYTCVVTYKNENISASAELTVLVPPWFLNHPSNLYAYESMDIEFECTVSGKPVPTVNWMKNGDVVIPSDYFQIVGGSNLRILGVVKSDEGFYQCVAENEAGNAQTSAQLIVPKPAIPSSSVLPSAPRDVVPVLVSSRFVRLSWRPPAEAKGNIQTFTVFFSREGDNRERALNTTQPGSLQLTVGNLKPEAMYTFRVVAYNEWGPGESSQPIKVATQPELQVPGPVENLQAVSTSPTSILITWEPPAYANGPVQGYRLFCTEVSTGKEQNIEVDGLSYKLEGLKKFTEYSLRFLAYNRYGPGVSTDDITVVTLSDVPSAPPQNVSLEVVNSRSIKVSWLPPPSGTQNGFITGYKIRHRKTTRRGEMETLEPNNLWYLFTGLEKGSQYSFQVSAMTVNGTGPPSNWYTAETPENDLDESQVPDQPSSLHVRPQTNCIIMSWTPPLNPNIVVRGYIIGYGVGSPYAETVRVDSKQRYYSIERLESSSHYVISLKAFNNAGEGVPLYESATTRSITDPTDPVDYYPLLDDFPTSVPDLSTPMLPPVGVQAVALTHDAVRVSWADNSVPKNQKTSEVRLYTVRWRTSFSASAKYKSEDTTSLSYTATGLKPNTMYEFSVMVTKNRRSSTWSMTAHATTYEAAPTSAPKDFTVITREGKPRAVIVSWQPPLEANGKITAYILFYTLDKNIPIDDWIMETISGDRLTHQIMDLNLDTMYYFRIQARNSKGVGPLSDPILFRTLKVEHPDKMANDQGRHGDGGYWPVDTNLIDRSTLNEPPIGQMHPPHGSVTPQKNSNLLVIIVVTVGVITVLVVVIVAVICTRRSSAQQRKKRATHSAGKRKGSQKDLRPPDLWIHHEEMEMKNIEKPSGTDPAGRDSPIQSCQDLTPVSHSQSETQLGSKSTSHSGQDTEEAGSSMSTLERSLAARRAPRAKLMIPMDAQSNNPAVVSAIPVPTLESAQYPGILPSPTCGYPHPQFTLRPVPFPTLSVDRGFGAGRSQSVSEGPTTQQPPMLPPSQPEHSSSEEAPSRTIPTACVRPTHPLRSFANPLLPPPMSAIEPKVPYTPLLSQPGPTLPKTHVKTASLGLAGKARSPLLPVSVPTAPEVSEESHKPTEDSANVYEQDDLSEQMASLEGLMKQLNAITGSAF

SEQ ID NO: 9 - DNA, 10386 bp, Homo sapiens
  CDS (39)..(8567)
  misc_feature (9521)..(9521): n is a, c, g, or t
attgaggactcggaaatgaggtccaagggtagccaaggatggctgcagcttcatatgatcagttgttaaagcaagttgaggcactgaagatggagaactcaaatcttcgacaagagctagaagataattccaatcatcttacaaaactggaaactgaggcatctaatatgaaggaagtacttaaacaactacaaggaagtattgaagatgaagctatggcttcttctggacagattgatttattagagcgtcttaaagagcttaacttagatagcagtaatttccctggagtaaaactgcggtcaaaaatgtccctccgttcttatggaagccgggaaggatctgtatcaagccgttctggagagtgcagtcctgttcctatgggttcatttccaagaagagggtttgtaaatggaagcagagaaagtactggatatttagaagaacttgagaaagagaggtcattgcttcttgctgatcttgacaaagaagaaaaggaaaaagactggtattacgctcaacttcagaatctcactaaaagaatagatagtcttcctttaactgaaaatttttccttacaaacagatatgaccagaaggcaattggaatatgaagcaaggcaaatcagagttgcgatggaagaacaactaggtacctgccaggatatggaaaaacgagcacagcgaagaatagccagaattcagcaaatcgaaaaggacatacttcgtatacgacagcttttacagtcccaagcaacagaagcagagaggtcatctcagaacaagcatgaaaccggctcacatgatgctgagcggcagaatgaaggtcaaggagtgggagaaatcaacatggcaacttctggtaatggtcagggttcaactacacgaatggaccatgaaacagccagtgttttgagttctagtagcacacactctgcacctcgaaggctgacaagtcatctgggaaccaaggtggaaatggtgtattcattgttgtcaatgcttggtactcatgataaggatgatatgtcgcgaactttgctagctatgtctagctcccaagacagctgtatatccatgcgacagtctggatgtcttcctctcctcatccagcttttacatggcaatgacaaagactctgtattgttgggaaattcccggggcagtaaagaggctcgggccagggccagtgcagcactccacaacatcattcactcacagcctgatgacaagagaggcaggcgtgaaatccgagtccttcatcttttggaacagatacgcgcttactgtgaaacctgttgggagtggcaggaagctcatgaaccaggcatggaccaggacaaaaatccaatgccagctcctgttgaacatcagatctgtcctgctgtgtgtgttctaatgaaactttcatttgatgaagagcatagacatgcaatgaatgaactagggggactacaggccattgcagaattattgcaagtggactgtgaaatgtacgggcttactaatgaccactacagtattacactaagacgatatgctggaatggctttgacaaacttgacttttggagatgtagccaacaaggctacgctatgctctatgaaaggctgcatgagagcacttgtggcccaactaaaatctgaaagtgaagacttacagcaggttattgcaagtgttttgaggaatttgtcttggcgagcagatgtaaatagtaaaaagacgttgcgagaagttggaagtgtgaaagcattgatggaatgtgctttagaagttaaaaaggaatcaaccctcaaaagcgtattgagtgccttatggaatttgtcagcacattgcactgagaataaagctgatatatgtgctgtagatggtgcacttgcatttttggttggcactcttacttaccggagccagacaaacactttagccattattgaaagtggaggtgggatattacggaatgtgtccagcttgatagctacaaatgaggaccacaggcaaatcctaagagagaacaactgtctacaaactttattacaacacttaaaatctcatagtttgacaatagtcagtaatgcatgtggaactttgtggaatctctcagcaagaaatcctaaagaccaggaagcattatgggacatgggggcagttagcatgctcaagaacctcattcattcaaagcacaaaatgattgctatgggaagtgctgcagctttaaggaatctcatggcaaataggcctgcgaagtacaaggatgccaatattatgtctcctggctcaagcttgccatctcttcatgttaggaaacaaaaagccctagaagcagaattagatgctcagcacttatcagaaacttttgacaatatagacaatttaagtcccaaggcatctcatcgtagtaagcagagacacaagcaaagtctctatggtgattatgtttttgacaccaatcgacatgatgataataggtcagacaattttaatactggcaacatgactgtcctttcaccatatttgaatactacagtgttacccagctcctcttcatcaagaggaagcttagatagttctcgttctgaaaaagatagaagtttggagagagaacgcggaattggtctaggcaactaccatccagcaacagaaaatccaggaacttcttcaaagcgaggtttgcagatctccaccactgcagcccagattgccaaagtcatggaagaagtgtcagccattcatacctctcaggaagacagaagttctgggtctaccactgaattacattgtgtgacagatgagagaaatgcacttagaagaagctctgctgcccatacacattcaaacacttacaatttcactaagtcggaaaattcaaataggacatgttctatgccttatgccaaattagaatacaagagatcttcaaatgatagtttaaatagtgtcagtagtagtgatggttatggtaaaagaggtcaaatgaaaccctcgattgaatcctattctgaagatgatgaaagtaagttttgcagttatggtcaatacccagccgacctagcccataaaatacatagtgcaaatcatatggatgataatgatggagaactagatacaccaataaattatagtcttaaatattcagatgagcagttgaactctggaaggcaaagtccttcacagaatgaaagatgggcaagacccaaacacataatagaagatgaaataaaacaaagtgagcaaagacaatcaaggaatcaaagtacaacttatcctgtttatactgagagcactgatgataaacacctcaagttccaaccacattttggacagcaggaatgtgtttctccatacaggtcacggggagccaatggttcagaaacaaatcgagtgggttctaatcatggaattaatcaaaatgtaagccagtctttgtgtcaagaagatgactatgaagatgataagcctaccaattatagtgaacgttactctgaagaagaacagcatgaagaagaagagagaccaacaaattatagcataaaatataatgaagagaaacgtcatgtggatcagcctattgattatagtttaaaatatgccacagatattccttcatcacagaaacagtcattttcattctcaaagagttcatctggacaaagcagtaaaaccgaacatatgtcttcaagcagtgagaatacgtccacaccttcatctaatgccaagaggcagaatcagctccatccaagttctgcacagagtagaagtggtcagcctcaaaaggctgccacttgcaaagtttcttctattaaccaagaaacaatacagacttattgtgtagaagatactccaatatgtttttcaagatgtagttcattatcatctttgtcatcagctgaagatgaaataggatgtaatcagacgacacaggaagcagattctgctaataccctgcaaatagcagaaataaaagaaaagattggaactaggtcagctgaagatcctgtgagcgaagttccagcagtgtcacagcaccctagaaccaaatccagcagactgcagggttctagtttatcttcagaatcagccaggcacaaagctgttgaattttcttcaggagcgaaatctccctccaaaagtggtgctcagacacccaaaagtccacctgaacactatgttcaggagaccccactcatgtttagcagatgtacttctgtcagttcacttgatagttttgagagtcgttcgattgccagctccgttcagagtgaaccatgcagtggaatggtaagtggcattataagccccagtgatcttccagatagccctggacaaaccatgccaccaagcagaagtaaaacacctccaccacctcctcaaacagctcaaaccaagcgagaagtacctaaaaataaagcacctactgctgaaaagagagagagtggacctaagcaagctgcagtaaatgctgcagttcagagggtccaggttcttccagatgctgatactttattacattttgccacggaaagtactccagatggattttcttgttcatccagcctgagtgctctgagcctcgatgagccatttatacagaaagatgtggaattaagaataatgcctccagttcaggaaaatgacaatgggaatgaaacagaatcagagcagcctaaagaatcaaatgaaaaccaagagaaagaggcagaaaaaactattgattctgaaaaggacctattagatgattcagatgatgatgatattgaaatactagaagaatgtattatttctgccatgccaacaaagtcatcacgtaaagcaaaaaagccagcccagactgcttcaaaattacctccacctgtggcaaggaaaccaagtcagctgcctgtgtacaaacttctaccatcacaaaacaggttgcaaccccaaaagcatgttagttttacaccgggggatgatatgccacgggtgtattgtgttgaagggacacctataaacttttccacagctacatctctaagtgatctaacaatcgaatcccctccaaatgagttagctgctggagaaggagttagaggaggagcacagtcaggtgaatttgaaaaacgagataccattcctacagaaggcagaagtacagatgaggctcaaggaggaaaaacctcatctgtaaccatacctgaattggatgacaataaagcagaggaaggtgatattcttgcagaatgcattaattctgctatgcccaaagggaaaagtcacaagcctttccgtgtgaaaaagataatggaccaggtccagcaagcatctgcgtcgtcttctgcacccaacaaaaatcagttagatggtaagaaaaagaaaccaacttcaccagtaaaacctataccacaaaatactgaatataggacacgtgtaagaaaaaatgcagactcaaaaaataatttaaatgctgagagagttttctcagacaacaaagattcaaagaaacagaatttgaaaaataattccaaggacttcaatgataagctcccaaataatgaagatagagtcagaggaagttttgcttttgattcacctcatcattacacgcctattgaaggaactccttactgtttttcacgaaatgattctttgagttctctagattttgatgatgatgatgttgacctttccagggaaaaggctgaattaagaaaggcaaaagaaaataaggaatcagaggctaaagttaccagccacacagaactaacctccaaccaacaatcagctaataagacacaagctattgcaaagcagccaataaatcgaggtcagcctaaacccatacttcagaaacaatccacttttccccagtcatccaaagacataccagacagaggggcagcaactgatgaaaagttacagaattttgctattgaaaatactccagtttgcttttctcataattcctctctgagttctctcagtgacattgaccaagaaaacaacaataaagaaaatgaacctatcaaagagactgagccccctgactcacagggagaaccaagtaaacctcaagcatcaggctatgctcctaaatcatttcatgttgaagataccccagtttgtttctcaagaaacagttctctcagttctcttagtattgactctgaagatgacctgttgcaggaatgtataagctccgcaatgccaaaaaagaaaaagccttcaagactcaagggtgataatgaaaaacatagtcccagaaatatgggtggcatattaggtgaagatctgacacttgatttgaaagatatacagagaccagattcagaacatggtctatcccctgattcagaaaattttgattggaaagctattcaggaaggtgcaaattccatagtaagtagtttacatcaagctgctgctgctgcatgtttatctagacaagcttcgtctgattcagattccatcctttccctgaaatcaggaatctctctgggatcaccatttcatcttacacctgatcaagaagaaaaaccctttacaagtaataaaggcccacgaattctaaaaccaggggagaaaagtacattggaaactaaaaagatagaatctgaaagtaaaggaatcaaaggaggaaaaaaagtttataaaagtttgattactggaaaagttcgatctaattcagaaatttcaggccaaatgaaacagccccttcaagcaaacatgccttcaatctctcgaggcaggacaatgattcatattccaggagttcgaaatagctcctcaagtacaagtcctgtttctaaaaaaggcccaccccttaagactccagcctccaaaagccctagtgaaggtcaaacagccaccacttctcctagaggagccaagccatctgtgaaatcagaattaagccctgttgccaggcagacatcccaaataggtgggtcaagtaaagcaccttctagatcaggatctagagattcgaccccttcaagacctgcccagcaaccattaagtagacctatacagtctcctggccgaaactcaatttcccctggtagaaatggaataagtcctcctaacaaattatctcaacttccaaggacatcatcccctagtactgcttcaactaagtcctcaggttctggaaaaatgtcatatacatctccaggtagacagatgagccaacagaaccttaccaaacaaacaggtttatccaagaatgccagtagtattccaagaagtgagtctgcctccaaaggactaaatcagatgaataatggtaatggagccaataaaaaggtagaactttctagaatgtcttcaactaaatcaagtggaagtgaatctgatagatcagaaagacctgtattagtacgccagtcaactttcatcaaagaagctccaagcccaaccttaagaagaaaattggaggaatctgcttcatttgaatctctttctccatcatctagaccagcttctcccactaggtcccaggcacaaactccagttttaagtccttcccttcctgatatgtctctatccacacattcgtctgttcaggctggtggatggcgaaaactcccacctaatctcagtcccactatagagtataatgatggaagaccagcaaagcgccatgatattgcacggtctcattctgaaagtccttctagacttccaatcaataggtcaggaacctggaaacgtgagcacagcaaacattcatcatcccttcctcgagtaagcacttggagaagaactggaagttcatcttcaattctttctgcttcatcagaatccagtgaaaaagcaaaaagtgaggatgaaaaacatgtgaactctatttcaggaaccaaacaaagtaaagaaaaccaagtatccgcaaaaggaacatggagaaaaataaaagaaaatgaattttctcccacaaatagtacttctcagaccgtttcctcaggtgctacaaatggtgctgaatcaaagactctaatttatcaaatggcacctgctgtttctaaaacagaggatgtttgggtgagaattgaggactgtcccattaacaatcctagatctggaagatctcccacaggtaatactcccccggtgattgacagtgtttcagaaaaggcaaatccaaacattaaagattcaaaagataatcaggcaaaacaaaatgtgggtaatggcagtgttcccatgcgtaccgtgggtttggaaaatcgcctgaactcctttattcaggtggatgcccctgaccaaaaaggaactgagataaaaccaggacaaaataatcctgtccctgtatcagagactaatgaaagttctatagtggaacgtaccccattcagttctagcagctcaagcaaacacagttcacctagtgggactgttgctgccagagtgactccttttaattacaacccaagccctaggaaaagcagcgcagatagcacttcagctcggccatctcagatcccaactccagtgaataacaacacaaagaagcgagattccaaaactgacagcacagaatccagtggaacccaaagtcctaagcgccattctgggtcttaccttgtgacatctgtttaaaagagaggaagaatgaaactaagaaaattctatgttaattacaactgctatatagacattttgtttcaaatgaaactttaaaagactgaaaaattttgtaaataggtttgattcttgttagagggtttttgttctggaagccatatttgatagtatactttgtcttcactggtcttattttgggaggcactcttgatggttaggaaaaaaatagtaaagccaagtatgtttgtacagtatgttttacatgtatttaaagtagcatcccatcccaacttcctttaattattgcttgtcttaaaataatgaacactacagatagaaaatatgatatattgctgttatcaatcatttctagattataaactgactaaacttacatcagggaaaaattggtatttatgcaaaaaaaaatgtttttgtccttgtgagtccatctaacatcataattaatcatgtggctgtgaaattcacagtaatatggttcccgatgaacaagctttacccagcctgtttgctttactgcatgaatgaaactgatggttcaatttcagaagtaatgattaacagttatgtggtcacatgatgtgcatagagatagctacagtgtaataatttacactattttgtgctccaaacaaaacaaaaatctgtgtaactgtaaaacattgaatgaaactattttacctgaactagattttatctgaaagtaggtagaatttttgctatgctgtaatttgttgtatattctggtatttgaggtgagatggctgctcttttattaatgagacatgaattgtgtctcaacagaaactaaatgaacatttcagaataaattattgctgtatgtaaactgttactgaaattggtatttgtttgaagggtcttgtttcacatttgtattaataattgtttaaaatgcctcttttaaaagcttatataaattttttncttcagcttctatgcattaagagtaaaattcctcttactgtaataaaaacaattgaagaagactgttgccacttaaccattccatgcgttggcacttatctattcctgaaattcttttatgtgattagctcatcttgatttttaacatttttccacttaaacttttttttcttactccactggagctcagtaaaagtaaattcatgtaatagcaatgcaagcagcctagcacagactaagcattgagcataataggcccacataatttcctctttcttaatattatagaaattctgtacttgaaattgattcttagacattgcagtctcttcgaggctttacagtgtaaactgtcttgccccttcatcttcttgttgcaactgggtctgacatgaacactttttatcaccctgtatgttagggcaagatctcagcagtgaagtataatcagcactttgccatgctcagaaaattcaaatcacatggaactttagaggtagatttaatacgattaagatattcagaagtatattttagaatccctgcctgttaaggaaactttatttgtggtaggtacagttctggggtacatgttaagtgtccccttatacagtggagggaagtcttccttcctgaaggaaaataaactgacacttattaactaagataatttacttaatatatcttccctgatttgttttaaaagatcagagggtgactgatgatacatgcatacatatttgttgaataaatgaaaatttatttttagtgataagattcatacactctgtatttggggagagaaaacctttttaagcatggtggggcactcagataggagtgaatacacctacctggtggtcat

SEQ ID NO: 10 - protein, 2843 aa, Homo sapiens
MAAASYDQLLKQVEALKMENSNLRQELEDNSNHLTKLETEASNMKEVLKQLQGSIEDEAMASSGQIDLLERLKELNLDSSNFPGVKLRSKMSLRSYGSREGSVSSRSGECSPVPMGSFPRRGFVNGSRESTGYLEELEKERSLLLADLDKEEKEKDWYYAQLQNLTKRIDSLPLTENFSLQTDMTRRQLEYEARQIRVAMEEQLGTCQDMEKRAQRRIARIQQIEKDILRIRQLLQSQATEAERSSQNKHETGSHDAERQNEGQGVGEINMATSGNGQGSTTRMDHETASVLSSSSTHSAPRRLTSHLGTKVEMVYSLLSMLGTHDKDDMSRTLLAMSSSQDSCISMRQSGCLPLLIQLLHGNDKDSVLLGNSRGSKEARARASAALHNIIHSQPDDKRGRREIRVLHLLEQIRAYCETCWEWQEAHEPGMDQDKNPMPAPVEHQICPAVCVLMKLSFDEEHRHAMNELGGLQAIAELLQVDCEMYGLTNDHYSITLRRYAGMALTNLTFGDVANKATLCSMKGCMRALVAQLKSESEDLQQVIASVLRNLSWRADVNSKKTLREVGSVKALMECALEVKKESTLKSVLSALWNLSAHCTENKADICAVDGALAFLVGTLTYRSQTNTLAIIESGGGILRNVSSLIATNEDHRQILRENNCLQTLLQHLKSHSLTIVSNACGTLWNLSARNPKDQEALWDMGAVSMLKNLIHSKHKMIAMGSAAALRNLMANRPAKYKDANIMSPGSSLPSLHVRKQKALEAELDAQHLSETFDNIDNLSPKASHRSKQRHKQSLYGDYVFDTNRHDDNRSDNFNTGNMTVLSPYLNTTVLPSSSSSRGSLDSSRSEKDRSLERERGIGLGNYHPATENPGTSSKRGLQISTTAAQIAKVMEEVSAIHTSQEDRSSGSTTELHCVTDERNALRRSSAAHTHSNTYNFTKSENSNRTCSMPYAKLEYKRSSNDSLNSVSSSDGYGKRGQMKPSIESYSEDDESKFCSYGQYPADLAHKIHSANHMDDNDGELDTPINYSLKYSDEQLNSGRQSPSQNERWARPKHIIEDEIKQSEQRQSRNQSTTYPVYTESTDDKHLKFQPHFGQQECVSPYRSRGANGSETNRVGSNHGINQNVSQSLCQEDDYEDDKPTNYSERYSEEEQHEEEERPTNYSIKYNEEKRHVDQPIDYSLKYATDIPSSQKQSFSFSKSSSGQSSKTEHMSSSSENTSTPSSNAKRQNQLHPSSAQSRSGQPQKAATCKVSSINQETIQTYCVEDTPICFSRCSSLSSLSSAEDEIGCNQTTQEADSANTLQIAEIKEKIGTRSAEDPVSEVPAVSQHPRTKSSRLQGSSLSSESARHKAVEFSSGAKSPSKSGAQTPKSPPEHYVQETPLMFSRCTSVSSLDSFESRSIASSVQSEPCSGMVSGIISPSDLPDSPGQTMPPSRSKTPPPPPQTAQTKREVPKNKAPTAEKRESGPKQAAVNAAVQRVQVLPDADTLLHFATESTPDGFSCSSSLSALSLDEPFIQKDVELRIMPPVQENDNGNETESEQPKESNENQEKEAEKTIDSEKDLLDDSDDDDIEILEECIISAMPTKSSRKAKKPAQTASKLPPPVARKPSQLPVYKLLPSQNRLQPQKHVSFTPGDDMPRVYCVEGTPINFSTATSLSDLTIESPPNELAAGEGVRGGAQSGEFEKRDTIPTEGRSTDEAQGGKTSSVTIPELDDNKAEEGDILAECINSAMPKGKSHKPFRVKKIMDQVQQASASSSAPNKNQLDGKKKKPTSPVKPIPQNTEYRTRVRKNADSKNNLNAERVFSDNKDSKKQNLKNNSKDFNDKLPNNEDRVRGSFAFDSPHHYTPIEGTPYCFSRNDSLSSLDFDDDDVDLSREKAELRKAKENKESEAKVTSHTELTSNQQSANKTQAIAKQPINRGQPKPILQKQSTFPQSSKDIPDRGAATDEKLQNFAIENTPVCFSHNSSLSSLSDIDQENNNKENEPIKETEPPDSQGEPSKPQASGYAPKSFHVEDTPVCFSRNSSLSSLSIDSEDDLLQECISSAMPKKKKPSRLKGDNEKHSPRNMGGILGEDLTLDLKDIQRPDSEHGLSPDSENFDWKAIQEGANSIVSSLHQAAAAACLSRQASSDSDSILSLKSGISLGSPFHLTPDQEEKPFTSNKGPRILKPGEKSTLETKKIESESKGIKGGKKVYKSLITGKVRSNSEISGQMKQPLQANMPSISRGRTMIHIPGVRNSSSSTSPVSKKGPPLKTPASKSPSEGQTATTSPRGAKPSVKSELSPVARQTSQIGGSSKAPSRSGSRDSTPSRPAQQPLSRPIQSPGRNSISPGRNGISPPNKLSQLPRTSSPSTASTKSSGSGKMSYTSPGRQMSQQNLTKQTGLSKNASSIPRSESASKGLNQMNNGNGANKKVELSRMSSTKSSGSESDRSERPVLVRQSTFIKEAPSPTLRRKLEESASFESLSPSSRPASPTRSQAQTPVLSPSLPDMSLSTHSSVQAGGWRKLPPNLSPTIEYNDGRPAKRHDIARSHSESPSRLPINRSGTWKREHSKHSSSLPRVSTWRRTGSSSSILSASSESSEKAKSEDEKHVNSISGTKQSKENQVSAKGTWRKIKENEFSPTNSTSQTVSSGATNGAESKTLIYQMAPAVSKTEDVWVRIEDCPINNPRSGRSPTGNTPPVIDSVSEKANPNIKDSKDNQAKQNVGNGSVPMRTVGLENRLNSFIQVDAPDQKGTEIKPGQNNPVPVSETNESSIVERTPFSSSSSSKHSSPSGTVAARVTPFNYNPSPRKSSADSTSARPSQIPTPVNNNTKKRDSKTDSTESSGTQSPKRHSGSYLVTSV

SEQ ID NO: 11 - DNA, 21 bp, Artificial sequence
  MCC probe/primer
cactgccccggaacggggcgc

SEQ ID NO: 12 - DNA, 21 bp, Artificial sequence
  MCC probe/primer
ctccgccgctgccgcagctgc

SEQ ID NO: 13 - DNA, 22 bp, Artificial sequence
  MCC probe/primer
caggaggctcggtggcgcggcg

SEQ ID NO: 14 - DNA, 20 bp, Artificial sequence
  MCC probe/primer
ctccgccgctgccgcagctg

SEQ ID NO: 15 - DNA, 19 bp, Artificial sequence
  MCC probe/primer
cgcgccgcgccaccgagcc

SEQ ID NO: 16 - DNA, 22 bp, Artificial sequence
  MCC probe/primer
tttcacggacgagccattgctg

SEQ ID NO: 17 - DNA, 595 bp, Artificial sequence
  bisulphite-treated methylated MCC gene promoter
gtggttagtagttagatagttgtgtttaattagaaattatttataattttagacgattcgaaggggaaatttcggcgtgaagtgtagtttcgtttcggttttttttagttttttttttttttttggaattcgaggcgcggatttttttcgttttatcgttttagttttttcgggagttcgtcggtgttttttagggtgtcggttcgtgttgggaagtgttttttattttggtaatggggggcggcgaggcaccgtaggagtggcgaggcggcgcccagggtggtattgtttcggaacggggcgttgggtgcgcgcgggagggttttcgcgcgggtttcgtcgttgtcgtagttgcgagcgcgtcgcgttatcgagttttttgtagtaatggttcgttcgtgaaacgcgagttacggttgttttttttaagagtgtttgtatttttcgtttgcgtttcgtaattgttttgggtgaaaatggttgtttagattaaaatgtggtagaagggattaagtagtggatattgagtttgtgaagtttaatttttaagttttgagatttgggggattgagagtttagttttgaaaagtgtatt

SEQ ID NO: 18 - DNA, 21 bp, Artificial sequence
  methylated sequence-specific MCC probe/primer
tattgtttcggaacggggcgt

SEQ ID NO: 19 - DNA, 21 bp, Artificial sequence
  methylated sequence-specific MCC probe/primer
tttcgtcgttgtcgtagttgc

SEQ ID NO: 20 - DNA, 22 bp, Artificial sequence
  methylated sequence-specific MCC probe/primer
caaaaaactcgataacgcgacg

SEQ ID NO: 21 - DNA, 20 bp, Artificial sequence
  methylated sequence-specific MCC probe/primer
tttcgtcgttgtcgtagttg

SEQ ID NO: 22 - DNA, 19 bp, Artificial sequence
  methylated sequence-specific MCC probe/primer
cgcgtcgcgttatcgagtt

SEQ ID NO: 23 - DNA, 21 bp, Artificial sequence
  methylated sequence-specific MCC probe/primer
ttcacgaacgaaccattacta

SEQ ID NO: 24 - DNA, 595 bp, Artificial sequence
  bisulphite-treated MCC promoter from healthy control
gtggttagtagttagatagttgtgtttaattagaaattatttataattttagatgatttgaaggggaaattttggtgtgaagtgtagttttgttttggttttttttagttttttttttttttttggaatttgaggtgtggattttttttgttttattgttttagtttttttgggagtttgttggtgttttttagggtgttggtttgtgttgggaagtgttttttattttggtaatggggggcggcgaggcaccgtaggagtggcgaggcggcgcccagggtggtattgttttggaatggggtgttgggtgtgtgtgggagggttttcgtgtgggttttgttgttgttgtagttgtgagtgtgttgtgttattgagttttttgtagtaatggtttgtttgtgaaatgtgagttatggttgttttttttaagagtgtttgtattttttgtttgtgttttgtaattgttttgggtgaaaatggttgtttagattaaaatgtggtagaagggattaagtagtggatattgagtttgtgaagtttaatttttaagttttgagatttgggggattgagagtttagttttgaaaagtgtatt

SEQ ID NO: 25 - DNA, 21 bp, Artificial sequence
  non-methylated sequence-specific MCC probe/primer
tattgtttgggaatggggtgt

SEQ ID NO: 26 - DNA, 21 bp, Artificial sequence
  non-methylated sequence-specific MCC probe/primer
ttttgttgttgttgtagttgt

SEQ ID NO: 27 - DNA, 22 bp, Artificial sequence
  non-methylated sequence-specific MCC probe/primer
caaaaaactcaataacacaaca

SEQ ID NO: 28 - DNA, 20 bp, Artificial sequence
  non-methylated sequence-specific MCC probe/primer
ttttgttgttgttgtagttg

SEQ ID NO: 29 - DNA, 19 bp, Artificial sequence
  non-methylated sequence-specific MCC probe/primer
tgtgttgtgttattgagtt

SEQ ID NO: 30 - DNA, 21 bp, Artificial sequence
  non-methylated sequence-specific MCC probe/primer
ttcacaaacaaaccattacta

SEQ ID NO: 31 - DNA, 21 bp, Artificial sequence
  sequencing primer MCC-promF
ggttagtagttagatagttgt

SEQ ID NO: 32 - DNA, 20 bp, Artificial sequence
  sequencing primer MCC-promR
tacttaatcccttctaccac

SEQ ID NO: 33 - DNA, 20 bp, Artificial sequence
  primer MCC-cDNAF1
ctgggtgaaaatggctgtct

SEQ ID NO: 34 - DNA, 20 bp, Artificial sequence
  primer MCC-cDNAR1
gttccctctctgtttgctgg

SEQ ID NO: 35 - DNA, 24 bp, Artificial sequence
  methylated p16 primer
ttattagagggtggggcggatcgc

SEQ ID NO: 36 - DNA, 21 bp, Artificial sequence
  methylated 16 primer
gaccccgaaccgcgaccgtaa

SEQ ID NO: 37 - DNA, 24 bp, Artificial sequence
  non-methylated p16 primer
ttattagagggtggggtggattgt

SEQ ID NO: 38 - DNA, 22 bp, Artificial sequence
  non-methylated p16 primer
cacctaaatcaacctccaacca

SEQ ID NO: 39 - DNA, 24 bp, Artificial sequence
  methylated HPP1 primer
gttatcgtcgtcgtttttgttgtc

SEQ ID NO: 40 - DNA, 23 bp, Artificial sequence
  methylated HPP1 primer
gacttccgaaaaacacaaaatcg

SEQ ID NO: 41 - DNA, 29 bp, Artificial sequence
  methylated HPP1 amplicon probe
ccgaacaacgaactactaaacatcccgcg

SEQ ID NO: 42 - DNA, 21 bp, Artificial sequence
  methylated hMLH1 primer
taatctatcgccgcctcatcg

SEQ ID NO: 43 - DNA, 37 bp, Artificial sequence
  methylated hMLH1 primer
tcgttatatatcgttcgtagtattcgtgtttagtttc

SEQ ID NO: 44 - DNA, 24 bp, Artificial sequence
  methylated hMLH amplicon probe
cgaacgcgacgtcaaacgccacta

SEQ ID NO: 45 - DNA, 23 bp, Artificial sequence
  methylated MGMT primer
tttcgacgttcgtaggttttcgc

SEQ ID NO: 46 - DNA, 22 bp, Artificial sequence
  methylated MGMT primer
gcactcttccgaaaacgaaacg